SEQUENCE LISTING (63 sequences)

SEQ ID NO: 1 - DNA, 30 bp, synthetic construct
  source 1..30: mol_type = other DNA; note = TSO primer
  misc_feature 28: note = n = riboguanosine (rG)
  misc_feature 29: note = n = riboguanosine (rG)
  misc_feature 30: note = n = Locked nucleic acid (LNA)-modified G (+G)
aagcagtggtatcaacgcagagtacatnnn

SEQ ID NO: 2 - DNA, 23 bp, synthetic construct
  source 1..23: mol_type = other DNA; note = ISPCR
aagcagtggtatcaacgcagagt

SEQ ID NO: 3 - DNA, 33 bp, synthetic construct
  source 1..33: mol_type = other DNA; note = 3'-P2
gtgactggagttcagacgtgtgctcttccgatc

SEQ ID NO: 4 - DNA, 45 bp, synthetic construct
  source 1..45: mol_type = other DNA; note = short universal primer
aatgatacggcgaccaccgagatctacactctttccctacacgac

SEQ ID NO: 5 - DNA, 21 bp, synthetic construct
  source 1..21: mol_type = other DNA; note = illunima-QP2
caagcagaagacggcatacga

SEQ ID NO: 6 - DNA, 22 bp, synthetic construct
  source 1..22: mol_type = other DNA; note = POU5F1:Forword
caaactgaggtgcctgcccttc

SEQ ID NO: 7 - DNA, 25 bp, synthetic construct
  source 1..25: mol_type = other DNA; note = POU5F1:Reverse
attgaacttcaccttccctccaacc

SEQ ID NO: 8 - DNA, 20 bp, synthetic construct
  source 1..20: mol_type = other DNA; note = NANOG:Forword
catctgctgagaccctcgac

SEQ ID NO: 9 - DNA, 20 bp, synthetic construct
  source 1..20: mol_type = other DNA; note = NANOG:Reverse
gggcttgtggaagaatcagg

SEQ ID NO: 10 - DNA, 22 bp, synthetic construct
  source 1..22: mol_type = other DNA; note = SOX2:Forword
catcaacggtacactgcctctc

SEQ ID NO: 11 - DNA, 23 bp, synthetic construct
  source 1..23: mol_type = other DNA; note = SOX2:Reverse
actctcctcccatttccctcttt

SEQ ID NO: 12 - DNA, 20 bp, synthetic construct
  source 1..20: mol_type = other DNA; note = REX1(ZFP42):Forword
gggatcacgtgtgtgcagaa

SEQ ID NO: 13 - DNA, 20 bp, synthetic construct
  source 1..20: mol_type = other DNA; note = REX1(ZFP42):Reverse
ttcctgcgacagccttcaaa

SEQ ID NO: 14 - DNA, 20 bp, synthetic construct
  source 1..20: mol_type = other DNA; note = CDH1:Forword
gacttctgccagaggaaccc

SEQ ID NO: 15 - DNA, 20 bp, synthetic construct
  source 1..20: mol_type = other DNA; note = CDH1:Reverse
cactggccccatgtgttagt

SEQ ID NO: 16 - DNA, 20 bp, synthetic construct
  source 1..20: mol_type = other DNA; note = IGF2:Forword
cgtgctgctatgctgcttac

SEQ ID NO: 17 - DNA, 20 bp, synthetic construct
  source 1..20: mol_type = other DNA; note = IGF2:Reverse
aagcagcactcttccacgat

SEQ ID NO: 18 - DNA, 20 bp, synthetic construct
  source 1..20: mol_type = other DNA; note = SNAI2:Forword
cccacaccctaccttgtgtc

SEQ ID NO: 19 - DNA, 20 bp, synthetic construct
  source 1..20: mol_type = other DNA; note = SNAI2:Reverse
tgacatccgagtgtgtctgc

SEQ ID NO: 20 - DNA, 20 bp, synthetic construct
  source 1..20: mol_type = other DNA; note = SRC:Forword
gccaacatcctggttggaga

SEQ ID NO: 21 - DNA, 20 bp, synthetic construct
  source 1..20: mol_type = other DNA; note = SRC:Reverse
atcccaaaggaccacacgtc

SEQ ID NO: 22 - DNA, 20 bp, synthetic construct
  source 1..20: mol_type = other DNA; note = WNT5A:Forword
cgcgaagacaggcatcaaag

SEQ ID NO: 23 - DNA, 20 bp, synthetic construct
  source 1..20: mol_type = other DNA; note = WNT5A:Reverse
cctatctgcatgaccctgcc

SEQ ID NO: 24 - DNA, 20 bp, synthetic construct
  source 1..20: mol_type = other DNA; note = GATA6:Forword
cactacttgtgcaaccgctg

SEQ ID NO: 25 - DNA, 20 bp, synthetic construct
  source 1..20: mol_type = other DNA; note = GATA6:Reverse
ttctgcggctttatgagggg

SEQ ID NO: 26 - DNA, 20 bp, synthetic construct
  source 1..20: mol_type = other DNA; note = HAND1:Forword
tgcgagtgcataccttctgt

SEQ ID NO: 27 - DNA, 20 bp, synthetic construct
  source 1..20: mol_type = other DNA; note = HAND1:Reverse
aggccctgaggggagtttat

SEQ ID NO: 28 - DNA, 20 bp, synthetic construct
  source 1..20: mol_type = other DNA; note = PAX6:Forword
tgtccaacggatgtgtgagt

SEQ ID NO: 29 - DNA, 19 bp, synthetic construct
  source 1..19: mol_type = other DNA; note = PAX6:Reverse
tctgtctcggatttcccaa

SEQ ID NO: 30 - DNA, 20 bp, synthetic construct
  source 1..20: mol_type = other DNA; note = CDH2:Forword
gcctcaagccaaccttacct

SEQ ID NO: 31 - DNA, 20 bp, synthetic construct
  source 1..20: mol_type = other DNA; note = CDH2:Reverse
agctcttgaggaaaaggccc

SEQ ID NO: 32 - DNA, 20 bp, synthetic construct
  source 1..20: mol_type = other DNA; note = ID2:Forword
tcgcaccccactattgtcag

SEQ ID NO: 33 - DNA, 20 bp, synthetic construct
  source 1..20: mol_type = other DNA; note = ID2:Reverse
ttcagaagcctgcaaggaca

SEQ ID NO: 34 - DNA, 21 bp, synthetic construct
  source 1..21: mol_type = other DNA; note = ID3:Forword
catcttcccatccagacagcc

SEQ ID NO: 35 - DNA, 20 bp, synthetic construct
  source 1..20: mol_type = other DNA; note = ID3:Reverse
gtcaagtgggcacgacaaag

SEQ ID NO: 36 - DNA, 20 bp, synthetic construct
  source 1..20: mol_type = other DNA; note = BMP2:Forword
gtctttcgggagcagacaca

SEQ ID NO: 37 - DNA, 20 bp, synthetic construct
  source 1..20: mol_type = other DNA; note = BMP2:Reverse
gtcaccaacctggtgtccaa

SEQ ID NO: 38 - DNA, 20 bp, synthetic construct
  source 1..20: mol_type = other DNA; note = EOMES:Forword
actcccatggacctccagaa

SEQ ID NO: 39 - DNA, 20 bp, synthetic construct
  source 1..20: mol_type = other DNA; note = EOMES:Reverse
tcgcttacaagcactggtgt

SEQ ID NO: 40 - DNA, 20 bp, synthetic construct
  source 1..20: mol_type = other DNA; note = T:Forword
gaagtacgtgaacggggagt

SEQ ID NO: 41 - DNA, 20 bp, synthetic construct
  source 1..20: mol_type = other DNA; note = T:Reverse
cacgatgtggattcgaggct

SEQ ID NO: 42 - DNA, 20 bp, synthetic construct
  source 1..20: mol_type = other DNA; note = LIN28A:Forword
tgccggcatctgtaaatggt

SEQ ID NO: 43 - DNA, 20 bp, synthetic construct
  source 1..20: mol_type = other DNA; note = LIN28A:Reverse
actctggtgcacaaagacgt

SEQ ID NO: 44 - DNA, 20 bp, synthetic construct
  source 1..20: mol_type = other DNA; note = C-MYC:Forword
atccaagaccaccaccactg

SEQ ID NO: 45 - DNA, 23 bp, synthetic construct
  source 1..23: mol_type = other DNA; note = C-MYC:Reverse
gttcacagcaacattcaggtaga

SEQ ID NO: 46 - DNA, 20 bp, synthetic construct
  source 1..20: mol_type = other DNA; note = ETV4:Forword
agaaccggccagctatgaac

SEQ ID NO: 47 - DNA, 20 bp, synthetic construct
  source 1..20: mol_type = other DNA; note = ETV4:Reverse
attgtccgggaaagccagag

SEQ ID NO: 48 - DNA, 20 bp, synthetic construct
  source 1..20: mol_type = other DNA; note = ETV5:Forword
tcagcacatgggttccagtc

SEQ ID NO: 49 - DNA, 20 bp, synthetic construct
  source 1..20: mol_type = other DNA; note = ETV5:Reverse
ccttcatggctgctggagaa

SEQ ID NO: 50 - DNA, 25 bp, synthetic construct
  source 1..25: mol_type = other DNA; note = BMP4:Forword
ttcattttaggagccattctgtagt

SEQ ID NO: 51 - DNA, 22 bp, synthetic construct
  source 1..22: mol_type = other DNA; note = BMP4:Reverse
tcctagcaggacttggcataat

SEQ ID NO: 52 - DNA, 20 bp, synthetic construct
  source 1..20: mol_type = other DNA; note = EF-1(alpha):Forword
aatgcggtgggatcgacaaa

SEQ ID NO: 53 - DNA, 20 bp, synthetic construct
  source 1..20: mol_type = other DNA; note = EF-1(alpha):Reverse
cacgctcacgttcagccttt

SEQ ID NO: 54 - DNA, 24 bp, synthetic construct
  source 1..24: mol_type = other DNA; note = TYR gene sgRNA targeting sequence: Forword
accgacctcagttccccttcaccg

SEQ ID NO: 55 - DNA, 24 bp, synthetic construct
  source 1..24: mol_type = other DNA; note = TYR gene sgRNA targeting sequence: Reverse
aaaccggtgaaggggaactgaggt

SEQ ID NO: 56 - DNA, 20 bp, synthetic construct
  source 1..20: mol_type = other DNA; note = TYR gene mutant site test primer: Forword
ctggactttccagacttccg

SEQ ID NO: 57 - DNA, 20 bp, synthetic construct
  source 1..20: mol_type = other DNA; note = TYR gene mutant site test primer: Reverse
gtgcagttgggtccctgaaa

SEQ ID NO: 58 - DNA, 20 bp, synthetic construct
  source 1..20: mol_type = other DNA; note = GFP test primer Forword
caacacccgcatcgagaagt

SEQ ID NO: 59 - DNA, 20 bp, synthetic construct
  source 1..20: mol_type = other DNA; note = GFP test primer Reverse
accacgaagctgtagtagcc

SEQ ID NO: 60 - DNA, 20 bp, synthetic construct
  source 1..20: mol_type = other DNA; note = NANOG knock-in 3' ARM test primer Forword
gcaacctccccttctacgag

SEQ ID NO: 61 - DNA, 20 bp, synthetic construct
  source 1..20: mol_type = other DNA; note = NANOG knock-in 3' ARM test primer Reverse
gtgaagcacacggtagggta

SEQ ID NO: 62 - DNA, 23 bp, synthetic construct
  source 1..23: mol_type = other DNA; note = NANOG knock-in 5' ARM test primer Forword
tgtccattgctgaagcatgtaat

SEQ ID NO: 63 - DNA, 23 bp, synthetic construct
  source 1..23: mol_type = other DNA; note = NANOG knock-in 5' ARM test primer Reverse
gaagttagtagctccgcttcctg

(19) United States
(12) Patent Application Publication
(10) Pub. No.: US 2024/0392240 A1
(43) Pub. Date: Nov. 28, 2024

(54) PIG EMBRYO-DERIVED PLURIPOTENT STEM CELLS AND USE THEREOF

(71) Applicant: CHINA AGRICULTURAL UNIVERSITY, Beijing (CN)

(72) Inventors: Jianyong HAN, Beijing (CN); Minglei ZHI, Beijing (CN); Jinying ZHANG, Beijing (CN)

(21) Appl. No.: 18/690,946

(22) PCT Filed: Sep. 7, 2022
(86) PCT No.: PCT/CN2022/117588

§ 371 Date: Mar. 11, 2024

(30) Foreign Application Priority Data
Sep. 10, 2021 (CN) 202111061392.9

(51) Int. Cl. C12N 5/0735; G01N 33/50

(52) U.S. Cl. CPC C12N 5/0606; G01N 33/5073; C12N 2510/00

(57) ABSTRACT

Pig embryo-derived pluripotent stem cells, having the characteristics and pluripotency of pig pre-gastrulation metaplasia epiblast cells. The solution is capable of stable passage, and is able to withstand multiple instances of consecutive gene editing and produce cloned pigs. The pig embryonic pluripotent stem cells create a new path for biological research, animal husbandry, and regenerative biomedicine.

### RELATED APPLICATIONS

The present application is a U.S. National Phase of International Application Number PCT/CN2022/117588 filed Sep. 7, 2022, which claims priority to Chinese Application Number 202111061392.9 filed Sep. 10, 2021.

### INCORPORATION BY REFERENCE

The sequence listing provided in the file entitled PUS124008PCT.xml, which is an Extensible Markup Language (XML) file that was created on Mar. 11, 2024, and which comprises 69,203 bytes, is hereby incorporated by reference in its entirety.

### TECHNICAL FIELD

The present invention relates to a pig embryo-derived pluripotent stem cell, which has the characteristics and pluripotency of a pig pre-gastrulation Epiblast cell. The solution is capable of stable passage and is able to withstand multiple instances of consecutive gene editing and produce cloned pigs. The pig embryonic pluripotent stem cell proposed by the present invention create a new path for biological research, animal husbandry, and regenerative biomedicine.

### BACKGROUND

Epiblast develops from the Inner cell mass (ICM) of the blastocyst and produces cell lineage of all somatic and germ cells, thus forming a normal embryo. In terms of pluripotency, Epiblast cells are an important source of pluripotent stem cells (PSCs) including mouse embryonic stem cells (ESCs) derived from naïve Epiblast (Boroviak et al., 2014; Evans and Kaufman, 1981; Martin, 1981; Ying et al., 2008) and Epiblast stem cells (EpiSCs) derived from the Epiblast at later developmental stages (Bao et al., 2009; Brons et al., 2007; Tesar et al., 2007). Recently, formative (or “intermediate”) pluripotent stem cells have been successfully obtained from mouse pre-gastrulation formative epiblast cells (Kinoshita et al., 2021; Wang et al., 2021; Yu et al. 2021). Human conventional ESCs are derived from ICM of the blastocyst while displaying similar features of primed pluripotency as mouse EpiSCs (Tesar et al., 2007; Thomson et al., 1998), and human PSCs with a naïve or formative pluripotency state may also be obtained (Gafni et al. 2013; Kinoshita et al. 2021; Theunissen et al. 2014).

Compared with many other animal models, pigs are more similar to humans in embryonic development, anatomy and physiology; so it follows that stable pig PSCs derived from epiblast cells should be excellent stable models for understanding the properties of human PSCs, potentially providing valuable information for modeling human development. The combination of stable pig PSCs and accurate multiple gene-editing technology will have large impacts on both biomedical research and animal breeding for agriculture.

However, despite extensive and ongoing attempts since the 1990s, no long-term passaged stable pig PSC lines have yet been established from ICMs or epiblasts at different stages or ectoderm isolated (Alberio et al., 2010; Choi et al., 2019; Gao et al., 2019; Haraguchi et al., 2012; Hou et al., 2016; Notarianni et al., 1990; Park et al., 2013; Vassiliev et al., 2010; Yuan et al., 2019; Zhang et al., 2019).

### SUMMARY OF THE INVENTION

In the present application, scRNA-seq of preimplantation pig embryos of all stages (each day during E0-E14) was conducted by the inventors to comprehensively profile the molecular basis of early pig embryonic development and pluripotency changes, basing on which stable pig pre-gastrulation Epiblast stem cell lines (designated pgEpiSCs) were further established. The pgEpiSCs provided by the present invention display pluripotency and the molecular properties of pre-gastrulation epiblasts, show elevated dome morphology, express pluripotency markers, maintain stability over long-term passages, and have capacities for both high-efficiency teratoma formation and differentiation into diverse cell types, and is also able to withstand multiple instances of consecutive gene editing.

Pluripotent Stem Cells Derived from Pig Embryos

In a first aspect, the present invention provides a pluripotent stem cell that has a pluripotency of pig pre-gastrulation Epiblast cells and expresses one or more pluripotency markers and one or more Epiblast markers and is capable of stable passage.

In some embodiments, the pluripotent stem cell is derived from pig embryo.

The pluripotent stem cell of the present invention expresses one or more pluripotency markers and one or more Epiblast markers.

In some embodiments, the one or more pluripotency markers are selected from POU5F1, NANOG, SOX2, SSEA1, SSEA4, TRA-1-81, TRA-1-60, and any combination thereof. In some embodiments, the pluripotent stem cell expresses one or more (e.g., at least 1, at least 2 or all) of POU5F1, NANOG, and SOX2. In some embodiments, the pluripotent stem cell expresses one or more (e.g., at least 1, at least 2, at least 3, or all) of SSEA1, SSEA4, TRA-1-81, and TRA-1-60. In some embodiments, the pluripotent stem cell expresses one or more (e.g., at least 1, at least 2, at least 3, at least 4, at least 5, at least 6, or all) of POU5F1, NANOG, SOX2, SSEA1, SSEA4, TRA-1-81, and TRA-1-60.

In some embodiments, the one or more Epiblast markers are selected from NANOG, TDGF1, ETV4, GDF3, NODAL, PRDM14, ETV5, CACHD1, and any combination thereof. In some embodiments, the pluripotent stem cell expresses one or more (e.g., at least 1, at least 2, at least 3, at least 4, or all) of NANOG, TDGF1, ETV4, GDF3, and NODAL. In some embodiments, the pluripotent stem cell expresses one or more (e.g., at least 1, at least 2, at least 3, at least 4, at least 5, at least 6, at least 7, or all) of NANOG, TDGF1, ETV4, GDF3, NODAL, PRDM14, ETV5, and CACHD1.

In some embodiments, the pluripotent stem cell of the present invention has no or low expression of at least one Hypoblast marker; alternatively, the pluripotent stem cell expresses at least one Hypoblast marker at a level that is lower than that in the pig embryonic Hypoblast cells of E8 to E10 (e.g., E8, E9, or E10), for example, having statistically significant difference.

In some embodiments, the Hypoblast marker is selected from IGF1, SRC, HNF4A, BMP2, SOX17, PDGFRA, NID2, RSPO3, GATA4, LAMA1 or any combination thereof.

In some embodiments, the pluripotent stem cell has no or low expression of one or more (e.g., at least 1, at least 2, or all) of HNF4A, SOX17, and GATA4.

In some embodiments, the pluripotent stem cell expresses at least one (e.g., at least 2 or all) of the following genes at a level that is lower than that in the pig embryonic Hypoblast cells of E8 to E10 (e.g., E8, E9, or E10): HNF4A, SOX17, and GATA4.

In some embodiments, the pluripotent stem cell of the present invention has no or low expression of at least one gastrulation marker; alternatively, the pluripotent stem cell expresses at least one gastrulation marker at a level that is lower than that in the pig embryonic Ectoderm cells of E11 to E14 (e.g., E11, E12, E13 or E14), which is of statistically significant difference.

In some embodiments, the gastrulation marker is selected from EOMES, WNT5A, BMP4, LEF1, HAND1 and any combination thereof.

In some embodiments, the pluripotent stem cell has no or low expression of one or more (e.g., at least 1, at least 2, at least 3, at least 4, or all) of EOMES, WNT5A, BMP4, LEF1, and HAND1.

In some embodiments, the pluripotent stem cell expresses at least one (e.g., at least 2, at least 3, at least 4, or all) of the following genes at a level that is lower than that in a pig embryonic Ectoderm cell of E11 to E14 (e.g., E11, E12, E13 or E14): EOMES, WNT5A, BMP4, LEF1, and HAND1.

In some embodiments, the pluripotent stem cell shows at least about a 2-fold increase in the expression level of at least one (e.g., at least 2, at least 5, at least 10, at least 15, at least 20, or all) gene selected from the following in relative to those in a stem cell derived from human embryo: ADPRM, FRG1, GAS2, HK3, NCAN, POUSF1B, ZFP2, CLDND2, CRK, DMP1, GATD3B, H3F3A, IRF8, ITGA4, KRT14, MPC1, MSH4, NDE1, PBX2, PRKY, RGL2, SOX10 and VHLL.

In some embodiments, the pluripotent stem cell shows at least about a 2-fold decrease in the expression level of at least one (e.g., at least 2, at least 5, at least 10, at least 15, or all) genes selected from the following in relative to those in a human embryonic stem cell: ABCC4, ADCY2, AK2, AKT1, BMP2, CD46, CDH3, DNM1, DPPA4, ETS1, GAB2, ID2, KDR, MMP24, TGFB1, VGLL3, ZNF195, and ZNF519.

In some embodiments, the s human embryonic stem cell for comparison to the pluripotent stem cell of the present invention refers to a conventional human embryonic stem cell (conventional hESC) or a human embryonic stem cell in a primed state.

As used herein, the term “conventional human embryonic stem cell (conventional hESC)” or “human embryonic stem cell in a primed state” have the same meaning. For definition of a primed state (primed pluripotency) see, for example, Weinberger, L., Ayyash, M., Novershtern, N. & Hanna, J. H. Dynamic stem cell states: naive to primed pluripotency in rodents and humans. Nat. Rev. Mol. Cell Biol. 17, 155-169, doi: 10.1038/nrm.2015.28 (2016).

In some embodiments, the pluripotent stem cell includes Genes with Co-variation Between regulatory potential score (RPS) and gene expression in relative to a porcine embryonic fibroblast (pEF), which is referred to as a co-variation gene. The co-variation gene is selected from at least one of the genes shown in Table 1 (e.g., at least 2, at least 5, at least 10, at least 15, at least 20, at least 25, at least 30, at least 35, at least 40, at least 45, at least 50, at least 55, at least 60, at least 65, at least 70 or all). In some embodiments, the co-variation genes are selected from at least one (e.g., at least 2, at least 5, at least 10, at least 15, at least 20, at least 25, at least 30, or all) of the following: METTL3, FGFR1, CYC1, ETV5, SOD1, KIF21B, DNMT3A, NOD2, SOX11, MCM7, ITGA4, MYB, UPP1, GSC, ZSCAN21, TFAP2C, ZIC2, LIN28B, ZIC5, HNF4G, MYCN, SALL4, CDH1, DNMT3B, ZFP42, SOX2, UTF1, PRDM14, LEFTY2, OTX2, and LIN28A. In some embodiments, the Genes with Co-variation Between Expression and RPS means that genes with higher RPS scores are generally upregulated (log 2 fold change [FC]>1, FDR<0.05) in pgEpiSCs in relative to pEFs. In some embodiments, the above-mentioned genes are identified using high-deep in situ high-throughput chromatin conformation capture (Hi-C) sequencing technology.

TABLE 1
Representative co-variant genes

| | Gene | | Gene |
|---|---|---|---|
| Ensembl ID | symbol | Ensembl ID | symbol |
| ENSSSCG00000003557 | LIN28A | ENSSSCG00000009429 | TNFSF11 |
| ENSSSCG00000007252 | DNMT3B | ENSSSCG00000002032 | SLC7A8 |
| ENSSSCG00000032299 | LEFTY2 | ENSSSCG00000009120 | ZGRF1 |
| ENSSSCG00000035444 | ZFP42 | ENSSSCG00000008618 | MYCN |
| ENSSSCG00000038188 | | ENSSSCG00000008465 | KCNG3 |
| ENSSSCG00000033478 | | ENSSSCG00000023861 | PFAS |
| ENSSSCG00000000252 | KRT8 | ENSSSCG00000032831 | BRI3BP |
| ENSSSCG00000040403 | NANOG | ENSSSCG00000006099 | ESRP1 |
| ENSSSCG00000000253 | KRT18 | ENSSSCG00000021259 | CDA |
| ENSSSCG00000008429 | EPCAM | ENSSSCG00000026718 | PLCH1 |
| ENSSSCG00000017934 | CLDN7 | ENSSSCG00000021941 | ZSCAN21 |
| ENSSSCG00000026894 | NFE2L3 | ENSSSCG00000007479 | SALL4 |
| ENSSSCG00000025652 | CDH1 | ENSSSCG00000000298 | PPP1R1A |
| ENSSSCG00000005063 | OTX2 | ENSSSCG00000007602 | BAIAP2L1 |
| ENSSSCG00000004679 | SORD | ENSSSCG00000026996 | ABCC4 |
| ENSSSCG00000031204 | | ENSSSCG00000037485 | SOX2 |
| ENSSSCG00000022739 | DSG2 | ENSSSCG00000009859 | TESC |
| ENSSSCG00000028804 | CCDC181 | ENSSSCG00000039169 | CER1 |
| ENSSSCG00000021207 | HESX1 | ENSSSCG00000031675 | |
| ENSSSCG00000040222 | | ENSSSCG00000006050 | |
| ENSSSCG00000036181 | | ENSSSCG00000009584 | SEMA4D |
| ENSSSCG00000039049 | MAP7 | ENSSSCG00000037744 | ZNF483 |
| ENSSSCG00000021307 | USP44 | ENSSSCG00000037612 | LIN28B |
| ENSSSCG00000007718 | CLDN4 | ENSSSCG00000011610 | NUP21 |
| ENSSSCG00000012003 | | ENSSSCG00000038138 | TFEC |
| ENSSSCG00000010771 | UTF1 | ENSSSCG00000016140 | FZD5 |
| ENSSSCG00000002361 | VRTN | ENSSSCG00000011257 | ACVR2B |
| ENSSSCG00000015246 | ST14 | ENSSSCG00000034904 | |
| ENSSSCG00000013469 | ZNF555 | ENSSSCG00000025838 | AP1M2 |
| ENSSSCG00000021767 | | ENSSSCG00000034478 | |
| ENSSSCG00000002884 | LSR | ENSSSCG00000014450 | TCOF1 |
| ENSSSCG00000006195 | PRDM14 | ENSSSCG00000011277 | CCK |
| ENSSSCG00000008902 | PPAT | ENSSSCG00000004621 | MYO5C |
| ENSSSCG00000017915 | VMO1 | ENSSSCG00000025672 | RAVER2 |
| ENSSSCG00000002456 | CHGA | ENSSSCG00000033197 | |
| ENSSSCG00000031347 | | ENSSSCG00000034739 | |
| ENSSSCG00000016841 | SLC1A3 | ENSSSCG00000004318 | RRAGD |
| ENSSSCG00000000766 | CECR2 | | |

In some embodiments, the expression level of at least one (e.g., at least 2, at least 5, at least 10, at least 15, at least 20, at least 25, at least 30, at least 35, at least 40, at least 45, at least 50, at least 55, at least 60, at least 65, at least 70, or all) gene selected from the genes in Table 1 is increased in the pluripotent stem cell in relative to that in porcine embryonic fibroblasts. In some embodiments, the expression level of at least one (e.g., at least 2, at least 5, at least 10, at least 15, at least 20, or all) gene selected from the following genes in the pluripotent stem cell is increased in relative to that in porcine embryonic fibroblasts: ZSCAN21, LIN28B, MYCN, SALLA, CDH1, DNMT3B, ZFP42, SOX2, UTF1, PRDM14, LEFTY2, OTX2, LIN28A, ACVR2B, HESX1, FZD5, PPP1R1A, VMO1, NANOG, KRT8, KRT18, and EPCAM.

In some embodiments, at least one (e.g., at least 2, at least 3, at least 4, at least 5, or all) transcription factor selected from the following interacts specifically with enhancers in the genome of the pluripotent stem cell in relative to that in a porcine embryonic fibroblast: OTX2, LIN28A, NANOG, PRDM14, SALL4, UTF1, ZFP42, CDH1, DNMT3B, and LEFTY2. In some embodiments, the specific interaction with an enhancer means that, as determined by high-deep in situ high-throughput chromatin conformation capture (Hi-C) sequencing that the transcription factor interacts with the enhancer and that the above-mentioned interaction is absent or relatively rare in porcine embryonic fibroblasts.

Herein, the expression may be monitored by measuring the level of a full-length mRNA, mRNA fragment, full-length protein or protein fragment of the gene. Therefore, in some embodiments, the expression level is of mRNA level or protein level.

In some embodiments, the expression is evaluated by analyzing the expression of mRNA transcripts of the gene, for example, as determined by transcriptome sequencing (e.g., scRNA-seq) or RT-PCR.

In other embodiments, the expression is evaluated by analyzing the expression of the protein product of the gene, for example, as determined by immunological detection.

In some embodiments, the expression of the pluripotency marker is evaluated at protein level, for example, as determined by immunological detection.

In some embodiments, the Epiblast markers, Hypoblast markers, gastrulation markers, differential genes compared with human embryonic stem cells, and differential genes compared with porcine embryonic fibroblasts are evaluated by the expression of mRNA transcripts, for example, by transcriptome sequencing (e.g., scRNA-seq) or RT-PCR.

In some embodiments, the pluripotent stem cell has the capacity to differentiate into cells of any one of endoderm, ectoderm, and mesoderm.

In some embodiments, the pluripotent stem cell is capable of forming a dome-shaped colonal morphology.

In some embodiments, the pluripotent stem cell is capable of stable passage for at least 10 times, at least 20 times, at least 30 times, at least 40 times, at least 50 times, at least 100 times, at least 150 times, at least 200 times or more.

In some embodiments, the pluripotent stem cell is derived from pre-gastrulation Epiblast of a pig embryo. In some embodiments, the pluripotent stem cell is derived from E8-E10 (e.g., E8, E9, or E10) pre-gastrulation Epiblast of a pig embryo. In some embodiments, the pluripotent stem cell is derived from E10 Epiblast of a pig embryo.

In some embodiments, the pluripotent stem cell is a cell line. In some embodiments, the pluripotent stem cell is an Epiblast stem cell.

In a second aspect, the present invention provides an isolated population of cells comprising the pluripotent stem cells of the present invention or any combination thereof.

In some embodiments, at least 50% (e.g., at least 60%, at least 70%, at least 75%, at least 80%, at least 85%, at least 90%, at least 95%, at least 98%, at least 99%, or about 100%) cells in the cell population are pluripotent stem cells of the present invention.

In some embodiments, at least 50% (e.g., at least 60%, at least 70%, at least 75%, at least 80%, at least 85%, at least 90%, at least 95%, at least 98%, at least 99%, or about 100%) cells in the cell population express one or more markers selected from the following: ETV5, NANOG, ETV4, NODAL, and GDF3. In some embodiments, about 100% of cells in the cell population express ETV5, NANOG, ETV4, NODAL, and GDF3. In some embodiments, the expression is evaluated by expression of mRNA transcripts, e.g., as determined by transcriptome sequencing (e.g., scRNA-seq) or RT-PCR.

### Applications

In another aspect, the present invention provides a genetically modified pluripotent stem cell, which is obtained by genetically modifying a pluripotent stem cell or population of cells of the present invention.

In some embodiments, the genetic modification comprises genome editing, including, for example, nucleic acid fragment deletion, gene modification, gene knockout, gene product expression alteration, repair of mutations, polynucleotide insertion, single base mutation, or any combination thereof.

In some embodiments, the genome editing comprises gene insertion, gene knock-in, gene knockout, gene mutation (e.g., a single base mutation), or any combination thereof.

In some embodiments, the genetically modified pluripotent stem cell comprises at least 2 (e.g., at least 3) kinds of genetic modifications.

In some embodiments, the genetically modified pluripotent stem cell withstands at least twice (e.g., at least 3) genetic modifications.

In another aspect, the present invention also provides a method for producing genetically modified pluripotent stem cells comprising genetically modifying a pluripotent stem cell or population of cells of the present invention. The present invention also provides use of the pluripotent stem cell or cell population of the present invention for producing genetically modified pluripotent stem cells.

In some embodiments, the genetic modification comprises genome editing, including, for example, nucleic acid fragment deletion, gene modification, gene knockout, gene product expression alteration, repair of mutations, polynucleotide insertion, single base mutation or any combination thereof.

In some embodiments, the genome editing comprises gene insertion, gene knock-in, gene knockout, gene mutation (e.g., single base mutation), or any combination thereof.

In another aspect, the present invention also provides a method for producing a pig embryo, comprising establishing an embryo by a nuclear transfer process in which a nucleus of the pluripotent stem cell, population of cells, or genetically modified pluripotent stem cell of the present invention is transferred into an enucleated porcine oocyte or egg cell. The present invention also provides a pig embryo produced by the method described above.

In another aspect, the present invention also provides a method for producing a cloned pig, comprising establishing an embryo by a nuclear transfer process in which a nucleus of the pluripotent stem cell, cell populations, or genetically-modified pluripotent stem cells of the present invention is transferred into an enucleated porcine oocyte or an egg cell; and transferring the embryo into a recipient host for gestation. The present invention also provides a cloned pig produced by the method described above.

In some embodiments, the method further comprises culturing the nuclear donor cells under the condition of inducing differentiation to slow down their proliferation prior to nuclear transplantation. In some embodiments, the inducing differentiation includes culturing in basal medium containing BMP4 (e.g., 10 ng/ml), SB431542 (e.g., 5 μM), and FGF2 (e.g., 10 ng/ml), e.g., for at least one week.

In another aspect, the present invention also provides use of the pluripotent stem cells, cell populations or genetically modified pluripotent stem cells of the present invention for producing a pig embryo.

In another aspect, the present invention also provides use of the pluripotent stem cells, cell populations or genetically modified pluripotent stem cells of the present invention for producing a cloned pig.

In another aspect, the present invention also provides a method for producing a cell, a tissue or an organ (for example, an organoid) in vitro, comprising culturing the pluripotent stem cell, cell population, or genetically modified pluripotent stem cell of the present invention under conditions that allow for differentiation of the pluripotent stem cell. The present invention also provides use of the pluripotent stem cell, cell population, or genetically modified pluripotent stem cell of the present invention for producing a cell, a tissue or an organ (for example, an organoid) in vitro.

In some embodiments, the cell comprises endodermal, ectodermal or mesodermal cells.

In another aspect, the present invention also provides use of the pluripotent stem cell, cell population, genetically modified pluripotent stem cell, or cells, tissues, or organs (for example, organoids) of the present invention produced by the pluripotent stem cell, the cell population or the genetically modified pluripotent stem cell in vitro as a disease model and/or a drug screening model, or for use in the preparation of a model of a disease and/or a model for drug screening.

Preparation of Pluripotent Stem Cells Derived from Pig Embryos

The pluripotent stem cell described in the first aspect or the cell population described in the second aspect of the present invention may be produced by the following methods:
1) providing an Epiblast derived from a pig embryo or an inner cell mass thereof;
2) culturing the Epiblast derived from a pig embryo or an inner cell mass thereof in a culture medium to obtain the pluripotent stem cell described in the first aspect or the cell population described in the second aspect.

In some embodiments, the culture medium contains:
A first component, which is IWR-1-endo;
A second component, which is selected from WH-4-023, A419259;
A third component, which is selected from fibroblast growth factors.

In some embodiments, the culture medium further contains:

A fourth component, which is selected from CHIR99021, WNT3a;

A fifth component, which is selected from TGF-β superfamily members; and

A sixth component, which is LIF.

In some embodiments, the second component is WH-4-023.

In some embodiments, the third component is selected from FGF2, FGF1.

In some embodiments, the third component is FGF2.

In some embodiments, the third component is recombinant human FGF2.

In some embodiments, the fourth component is CHIR99021.

In some embodiments, the fifth component is selected from Activin A, Nodal.

In some embodiments, the fifth component is Activin A.

In some embodiments, the fifth component is recombinant human Activin A.

In some embodiments, the sixth component is selected from recombinant human LIF, recombinant mouse LIF.

In some embodiments, the sixth component is recombinant human LIF.

In some embodiments, the concentration of the first component is 0.1-10 μM, such as 0.1 μM, 0.2 μM, 0.3 μM, 0.4 μM, 0.5 μM, 0.7 μM, 0.9 μM, 1.1 μM, 1.3 μM, 1.5 μM, 1.7 μM, 1.9 μM, 2.0 μM, 2.1 μM, 2.2 μM, 2.3 μM, 2.4 μM, 2.5 μM, 2.6 μM, 2.7 μM, 2.8 μM, 2.9 μM, 3.0 μM, 3.1 μM, 3.3 μM, 3.5 μM, 3.7 μM, 3.9 μM, 4.0 μM, 4.1 μM, 4.3 μM, 4.5 μM, 4.7 μM, 4.9 μM, 5.0 μM, 5.1 μM, 5.3 μM, 5.5 μM, 5.7 μM, 5.9 μM, 6.0 μM, 6.1 μM, 6.3 μM, 6.5 μM, 6.7 μM, 6.9 μM, 7.0 μM, 7.1 μM, 7.3 μM, 7.5 μM, 7.7 μM, 7.9 μM, 8.0 μM, 8.1 μM, 8.3 μM, 8.5 μM, 8.7 μM, 8.9 μM, 9.0 μM, 9.1 μM, 9.3 μM, 9.5 μM, 9.7 μM, 9.9 μM or 10 μM, alternatively, 0.1-0.2 μM, 0.2-0.3 μM, 0.3-0.4 μM, 0.4-0.5 μM, 0.5-0.7 μM, 0.7-0.9 μM, 0.9-1.1 μM, 1.1-1.3 μM, 1.3-1.5 μM, 1.5-1.7 μM, 1.7-1.9 μM, 1.9-2.0 μM, 2.0-2.1 μM, 2.1-2.3 μM, 2.3-2.5 μM, 2.5-2.7 μM, 2.7-2.9 μM, 2.9-3.0 μM, 3.0-3.1 μM, 3.1-3.3 μM, 3.3-3.5 μM, 3.5-3.7 μM, 3.7-3.9 μM, 3.9-4.0 μM, 4.0-4.1 μM, 4.1-4.3 μM, 4.3-4.5 μM, 4.5-4.7 μM, 4.7-4.9 μM, 4.9-5.0 μM, 5.0-5.5 μM, 5.5-6 μM, 6-6.5 μM, 6.5-7 μM, 7-7.5 μM, 7.5-8 μM, 8-8.5 μM, 8.5-9 μM, 9-9.5 μM or 9.5-10 μM.

In some embodiments, the concentration of the first component is 0.9-3 μM.

In some embodiments, the concentration of the first component is 1-3 μM, such as 1 μM, 1.1 μM, 1.3 μM, 1.5 μM, 1.7 μM, 1.9 μM, 2.0 μM, 2.1 μM, 2.2 μM, 2.3 μM, 2.4 μM, 2.5 μM, 2.6 μM, 2.7 μM, 2.8 μM, 2.9 μM or 3.0 μM, alternatively, 1-1.1 μM, 1.1-1.3 μM, 1.3-1.5 μM, 1.5-1.7 μM, 1.7-1.9 μM, 1.9-2.0 μM, 2.0-2.1 μM, 2.1-2.3 μM, 2.3-2.5 μM, 2.5-2.7 μM, 2.7-2.9 μM or 2.9-3.0 μM.

In some embodiments, the concentration of the first component is 2.5 μM.

In some embodiments, the concentration of the second component is 3 nM-30 μM, such as 3 nM, 4 ηM, 5 nM, 6 nM, 7 nM, 8 nM, 9 ηM, 0.01 μM, 0.03 μM, 0.05 μM, 0.07 μM, 0.1 μM, 0.2 μM, 0.3 μM, 0.4 μM, 0.5 μM, 0.6 μM, 0.7 μM, 0.8 μM, 0.9 μM, 1 μM, 1.1 μM, 1.2 μM, 1.3 μM, 1.4 μM, 1.5 μM, 1.6 μM, 1.7 μM, 1.8 μM, 1.9 μM, 2.0 μM, 2.1 μM, 2.2 μM, 2.3 μM, 2.4 μM, 2.5 μM, 2.6 μM, 2.7 μM, 2.8 μM, 2.9 μM, 3.0 μM, 3.1 μM, 3.3 μM, 3.5 μM, 3.7 μM, 3.9 μM, 4.0 μM, 4.1 μM, 4.3 μM, 4.5 μM, 4.7 μM, 4.9 μM, 5.0 μM, 7.0 μM, 10 μM, 13 μM, 15 μM, 17 μM, 20 μM, 23 μM, 25 μM, 27 μM or 30 μM, alternatively, 3-4 nM, 4-5 nM, 5-6 nM, 6-7 nM, 7-8 nM, 8-9 μM, 9-10 μM, 0.01-0.03 μM, 0.03-0.05 μM, 0.05-0.07 μM, 0.07-0.1 μM, 0.1-0.2 μM, 0.2-0.3 μM, 0.3-0.4 μM, 0.1-0.2 μM, 0.2-0.3 μM, 0.3-0.4 μM, 0.4-0.5 μM, 0.5-0.7 μM, 0.7-0.9 μM, 0.9-1.1 μM, 1.1-1.3 μM, 1.3-1.5 μM, 1.5-1.7 μM, 1.7-1.9 μM, 1.9-2.0 μM, 2.0-2.1 μM, 2.1-2.3 μM, 2.3-2.5 μM, 2.5-2.7 μM, 2.7-2.9 μM, 2.9-3.0 μM, 3.0-3.1 μM, 3.1-3.3 μM, 3.3-3.5 μM, 3.5-3.7 μM, 3.7-3.9 μM, 3.9-4.0 μM, 4.0-4.1 μM, 4.1-4.3 μM, 4.3-4.5 μM, 4.5-4.7 μM, 4.7-4.9 μM, 4.9-5.0 μM, 5.0-7.0 μM, 7-10 μM, 10-13 μM, 13-15 μM, 15-17 μM, 17-20 μM, 20-23 μM, 23-25 μM, 25-27 μM or 27-30 μM.

In some embodiments, the concentration of the second component is 0.01-5 μM, such as 0.01 μM, 0.03 μM, 0.05 μM, 0.07 μM, 0.1 μM, 0.2 μM, 0.3 μM, 0.4 μM, 0.5 μM, 0.6 μM, 0.7 μM, 0.8 μM, 0.9 μM, 1 μM, 1.1 μM, 1.2 μM, 1.3 μM, 1.4 μM, 1.5 μM, 1.6 μM, 1.7 μM, 1.8 μM, 1.9 μM, 2.0 μM, 2.1 μM, 2.2 μM, 2.3 μM, 2.4 μM, 2.5 μM, 2.6 μM, 2.7 μM, 2.8 μM, 2.9 μM, 3.0 μM, 3.1 μM, 3.3 μM, 3.5 μM, 3.7 μM, 3.9 μM, 4.0 μM, 4.1 μM, 4.3 μM, 4.5 μM, 4.7 μM, 4.9 μM or 5.0 μM, alternatively, 0.01-0.03 μM, 0.03-0.05 μM, 0.05-0.07 μM, 0.07-0.1 μM, 0.1-0.2 μM, 0.2-0.3 μM, 0.3-0.4 μM, 0.4-0.5 μM, 0.5-0.7 μM, 0.7-0.9 μM, 0.9-1.1 μM, 1.1-1.3 μM, 1.3-1.5 μM, 1.5-1.7 μM, 1.7-1.9 μM, 1.9-2.0 μM, 2.0-2.1 μM, 2.1-2.3 μM, 2.3-2.5 μM, 2.5-2.7 μM, 2.7-2.9 μM, 2.9-3.0 μM, 3.0-3.1 μM, 3.1-3.3 μM, 3.3-3.5 M, 3.5-3.7 μM, 3.7-3.9 μM, 3.9-4.0 μM, 4.0-4.1 μM, 4.1-4.3 μM, 4.3-4.5 μM, 4.5-4.7 μM, 4.7-4.9 μM or 4.9-5.0 μM.

In some embodiments, the concentration of the second component is 1 μM.

In some embodiments, the concentration of the third component is 0.01-100 ng/mL, such as 0.01 ng/mL, 0.1 ng/ml, 0.2 ng/mL, 0.3 ng/ml, 0.4 ng/ml, 0.5 ng/ml, 0.6 ng/ml, 0.7 ng/ml, 0.8 ng/ml, 0.9 ng/ml, 1 ng/ml, 1.5 ng/ml, 2 ng/ml, 3 ng/mL, 4 ng/mL, 5 ng/mL, 6 ng/ml, 7 ng/ml, 8 ng/mL, 9 ng/ml, 10 ng/ml, 11 ng/mL, 12 ng/ml, 13 ng/ml, 14 ng/ml, 15 ng/ml, 16 ng/ml, 17 ng/mL, 18 ng/ml, 19 ng/ml, 20 ng/ml, 25 ng/ml, 30 ng/mL, 35 ng/mL, 40 ng/mL, 45 ng/ml, 50 ng/mL, 55 ng/mL, 60 ng/mL, 65 ng/mL, 70 ng/ml, 75 ng/ml, 80 ng/ml, 85 ng/ml, 90 ng/mL, 95 ng/ml or 100 ng/mL, alternatively, 0.01-0.1 ng/ml, 0.1-0.2 ng/mL, 0.2-0.5 ng/ml, 0.5-1 ng/mL, 1-1.5 ng/ml, 1.5-2 ng/ml, 2-3 ng/ml, 3-4 ng/ml, 4-5 ng/mL, 5-6 ng/mL, 6-7 ng/ml, 7-8 ng/mL, 8-9 ng/ml, 9-10 ng/ml, 10-11 ng/ml, 11-12 ng/mL, 12-13 ng/ml, 13-14 ng/ml, 14-15 ng/ml, 15-16 ng/ml, 16-17 ng/ml, 17-18 ng/ml, 18-19 ng/mL, 19-20 ng/mL, 20-25 ng/ml, 25-30 ng/mL, 30-35 ng/ml, 35-40 ng/mL, 40-45 ng/mL, 45-50 ng/mL, 50-55 ng/mL, 55-60 ng/ml, 60-65 ng/ml, 65-70 ng/mL, 70-75 ng/ml, 75-80 ng/mL, 80-85 ng/ml, 85-90 ng/mL, 90-95 ng/ml or 95-100 ng/mL.

In some embodiments, the concentration of the third component is 1-100 ng/mL.

In some embodiments, the concentration of the third component is 10 ng/ml.

In some embodiments, the concentration of the fourth component is 0.0025 nM-3 μM, such as 0.0025 nM, 0.005 nM, 0.01 nM, 0.015 nM, 0.02 nM, 0.025 nM, 0.03 nM, 0.035 nM, 0.04 nM, 0.045 nM, 0.05 nM, 0.1 nM, 0.15 nM, 0.2 nM, 0.25 nM, 0.3 nM, 0.35 nM, 0.4 nM, 0.45 nM, 0.5 nM, 1 nM, 1.5 nM, 2 nM, 2.5 nM, 3 nM, 4 nM, 5 nM, 6 nM, 7 nM, 8 nM, 9 nM, 0.01 μM, 0.05 μM, 0.1 μM, 0.15 μM, 0.2 μM, 0.3 μM, 0.4 μM, 0.5 μM, 0.6 μM, 0.7 μM, 0.8 μM, 0.9 μM, 1 μM, 1.1 μM, 1.2 μM, 1.3 μM, 1.4 μM, 1.5 μM, 1.6 μM, 1.7 μM, 1.8 μM, 1.9 μM, 2.0 μM, 2.1 μM, 2.2 μM, 2.3 μM, 2.4 μM, 2.5 μM, 2.6 μM, 2.7 μM, 2.8 μM, 2.9 μm or 3.0 μM, alternatively, 0.0025-0.005 nM, 0.005-0.01 nM, 0.01-0.015 nM, 0.015-0.02 nM, 0.02-0.025 nM, 0.025-0.03 nM, 0.03-0.035 nM, 0.035-0.04 nM, 0.04-0.045 nM, 0.045-0.05 nM, 0.05-0.1 nM, 0.1-0.15 nM, 0.15-0.2 nM, 0.2-0.25 nM, 0.25-0.3 nM, 0.3-0.35 nM, 0.35-0.4 nM, 0.4-0.45 nM, 0.45-0.5 nM, 0.5-1 nM, 1-1.5 nM, 1.5-2 nM, 2-2.5 nM, 2.5-3 nM, 3-4 nM, 4-5 nM, 5-6 nM, 6-7 nM, 7-8 nM, 8-9 nM, 9-10 nM, 0.01-0.05 μM, 0.05-0.1 μM, 0.1-0.15 μM, 0.15-0.2 μM, 0.2-0.3 M, 0.3-0.4 μM, 0.4-0.5 μM, 0.5-0.7 μM, 0.7-0.9 μM, 0.9-1.1 μM, 1.1-1.3 μM, 1.3-1.5 μM, 1.5-1.7 μM, 1.7-1.9 μM, 1.9-2.0 μM, 2.0-2.1 μM, 2.1-2.3 μM, 2.3-2.5 μM, 2.5-2.7 μM, 2.7-2.9 μM or 2.9-3.0 M.

In some embodiments, the concentration of the fourth component is 0.01-3 μM, such as 0.01 μM, 0.05 μM, 0.1 μM, 0.15 μM, 0.2 μM, 0.3 μM, 0.4 μM, 0.5 μM, 0.6 μM, 0.7 μM, 0.8 μM, 0.9 μM, 1 μM, 1.1 μM, 1.2 μM, 1.3 μM, 1.4 μM, 1.5 μM, 1.6 μM, 1.7 μM, 1.8 μM, 1.9 μM, 2.0 μM, 2.1 μM, 2.2 μM, 2.3 μM, 2.4 μM, 2.5 μM, 2.6 μM, 2.7 μM, 2.8 μM, 2.9 μM or 3.0 μM, alternatively, 0.01-0.05 μM, 0.05-0.1 μM, 0.1-0.15 μM, 0.15-0.2 μM, 0.2-0.3 μM, 0.3-0.4 μM, 0.4-0.5 μM, 0.5-0.7 μM, 0.7-0.9 μM, 0.9-1.1 μM, 1.1-1.3 μM, 1.3-1.5 μM, 1.5-1.7 μM, 1.7-1.9 μM, 1.9-2.0 μM, 2.0-2.1 μM, 2.1-2.3 μM, 2.3-2.5 μM, 2.5-2.7 μM, 2.7-2.9 μM or 2.9-3.0 μM.

In some embodiments, the concentration of the fourth component is 1 μM.

In some embodiments, the concentration of the fifth component is 0.01-100 ng/mL, such as 0.01 ng/ml, 0.5 ng/ml, 1 ng/mL, 1.5 ng/mL, 2 ng/ml, 2.5 ng/ml, 3 ng/ml, 3.5 ng/ml, 4 ng/ml, 4.5 ng/ml, 5 ng/ml, 6 ng/ml, 7 ng/ml, 8 ng/ml, 9 ng/mL, 10 ng/mL, 11 ng/ml, 12 ng/ml, 13 ng/mL, 14 ng/ml, 15 ng/ml, 16 ng/ml, 17 ng/mL, 18 ng/ml, 19 ng/mL, 20 ng/ml, 21 ng/mL, 22 ng/ml, 23 ng/ml, 24 ng/mL, 25 ng/mL, 26 ng/ml, 27 ng/mL, 28 ng/mL, 29 ng/ml, 30 ng/ml, 31 ng/mL, 32 ng/mL, 33 ng/ml, 34 ng/mL, 35 ng/ml, 36 ng/ml, 37 ng/mL, 38 ng/ml, 39 ng 40 ng/ml, 41 ng/mL, 42 ng/mL, 43 ng/mL, 44 ng/ml, 45 ng/ml, 46 ng/ml, 47 ng/mL, 48 ng/ml, 49 ng/mL, 50 ng/ml, 55 ng/ml, 60 ng/ml, 65 ng/ml, 70 ng/ml, 75 ng/mL, 80 ng/ml, 85 ng/ml, 90 ng/mL, 95 ng/ml or 100 ng/mL, alternatively, 5-6 ng/ml, 6-7 ng/mL, 7-8 ng/ml, 8-9 ng/mL, 9-10 ng/ml, 10-11 ng/ml, 11-12 ng/ml, 12-13 ng/ml, 13-14 ng/mL, 14-15 ng/ml, 15-16 ng/ml, 16-17 ng/ml, 17-18 ng/mL, 18-19 ng/ml, 19-20 ng/ml, 20-21 ng/mL, 21-23 ng/ml, 23-25 ng/ml, 25-27 ng/ml, 27-29 ng/ml, 29-30 ng/ml, 30-31 ng/ml, 31-33 ng/ml, 33-35 ng/ml, 35-37 ng/mL, 37-39 ng/ml, 39-40 ng/ml, 40-41 ng/mL, 41-43 ng/mL, 43-45 ng/ml, 45-47 ng/mL, 47-49 ng/mL, 49-50 ng/mL, 50-55 ng/ml, 55-60 ng/mL, 60-65 ng/ml, 65-70 ng/ml, 70-75 ng/mL, 75-80 ng/mL, 80-85 ng/mL, 85-90 ng/ml, 90-95 ng/ml or 95-100 ng/mL.

In some embodiments, the concentration of the fifth component is 25 ng/mL.

In some embodiments, the concentration of the sixth component is 0.01-100 ng/mL, such as 0.01 ng/ml, 0.05 ng/ml, 0.1 ng/mL, 0.5 ng/ml, 0.7 ng/ml, 1 ng/ml, 2 ng/ml, 3 ng/ml, 4 ng/ml, 5 ng/ml, 6 ng/mL, 7 ng/ml, 8 ng/ml, 9 ng/mL, 10 ng/mL, 11 ng/ml, 12 ng/ml, 13 ng/mL, 14 ng/ml, 15 ng/ml, 16 ng/ml, 17 ng/ml, 18 ng/mL, 19 ng/mL, 20 ng/ml, 21 ng/ml, 22 ng/mL, 23 ng/ml, 24 ng/ml, 25 ng/mL, 26 ng/mL, 27 ng/ml, 28 ng/mL, 29 ng/ml, 30 ng/ml, 31 ng/ml, 32 ng/ml, 33 ng/mL, 34 ng/ml, 35 ng/ml, 36 ng/mL, 37 ng/ml, 38 ng/mL, 39 ng/mL, 40 ng/mL, 41 ng/ml, 42 ng/ml, 43 ng/ml, 44 ng/ml, 45 ng/ml, 46 ng/ml, 47 ng/mL, 48 ng/ml, 49 ng/ml, 50 ng/ml, 53 ng/ml, 55 ng/ml, 57 ng/ml, 60 ng/ml, 63 ng/ml, 65 ng/ml, 67 ng/mL, 70 ng/mL, 73 ng/mL, 75 ng/mL, 77 ng/ml, 80 ng/ml, 83 ng/mL, 85 ng/ml, 87 ng/ml, 90 ng/ml, 93 ng/mL, 95 ng/ml, 97 ng/mL or 100 ng/mL, alternatively, 0.01-0.05 ng/ml, 0.05-0.1 ng/ml, 0.1-0.5 ng/ml, 0.5-0.7 ng/ml, 0.7-1 ng/mL, 1-2 ng/ml, 2-3 ng/ml, 3-4 ng/mL, 4-5 ng/ml, 5-6 ng/ml, 6-7 ng/mL, 7-8 ng/mL, 8-9 ng/mL, 9-10 ng/ml, 10-11 ng/mL, 11-12 ng/ml, 12-13 ng/ml, 13-14 ng/mL, 14-15 ng/ml, 15-16 ng/ml, 16-17 ng/ml, 17-18 ng/ml, 18-19 ng/ml, 19-20 ng/ml, 20-21 ng/ml, 21-23 ng/ml, 23-25 ng/mL, 25-27 ng/mL, 27-29 ng/ml, 29-30 ng/ml, 30-31 ng/ml, 31-33 ng/ml, 33-35 ng/mL, 35-37 ng/ml, 37-39 ng/mL, 39-40 ng/mL, 40-41 ng/mL, 41-43 ng/ml, 43-45 ng/mL, 45-47 ng/mL, 47-49 ng/ml, 49-50 ng/mL, 50-53 ng/mL, 53-55 ng/mL, 55-57 ng/ml, 57-59 ng/mL, 59-60 ng/ml, 60-63 ng/mL, 63-65 ng/ng/ml, 65-67 ng/ml, 67-69 ng/ml, 69-70 ng/mL, 70-73 ng/mL, 73-75 ng/mL, 75-77 ng/mL, 77-79 ng/mL, 79-80 ng/ml, 80-83 ng/mL, 83-85 ng/ml, 85-87 ng/mL, 87-90 ng/mL, 90-93 ng/mL, 93-95 ng/mL, 95-97 ng/mL, 97-99 ng/ml or 99-100 ng/mL.

In some embodiments, the concentration of the sixth component is 1-100 ng/mL.

In some embodiments, the concentration of the sixth component is 10 ng/mL.

In some embodiments, the concentration ratio of the fourth component to the first component is 25:1-1:25, such as 25:1, 24:1, 23:1, 22:1, 21:1, 20:1, 19:1, 18:1, 17:1, 16:1, 15:1, 14:1, 13:1, 12:1, 11:1, 10:1, 9:1, 8:1, 7:1 1:1, 6:1, 5:1, 4:1, 3:1, 2:1, 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:21, 1:22, 1:23, 1:24 or 1:25, alternatively, 25:1-23:1, 23:1-21:1, 21:1-20:1, 20:1-19:1, 19:1-17:1, 17:1-15:1, 15:1-13:1, 13:1-11:1, 11:1-10:1, 10:1-9:1, 9:1-7:1, 7:1-5:1, 5:1-3:1, 3:1-1:1, 1:1-1:3, 1:3-1:5, 1:5-1:7, 1:7-1:9, 1:9-1:10, 1:10-1:11, 1:11-1:13, 1:13-1:15, 1:15-1:17, 1:17-1:19, 1:19-1:20, 1:20-1:21, 1:21-1:23 or 1:23-1:25.

In some embodiments, the concentration ratio of the fourth component to the first component is 2:3-1:3.

In some embodiments, the concentration ratio of the fourth component to the first component is 1:2-1:3.

In some embodiments, the medium contains:

| Name | Concentration |
|---|---|
| CHIR99021 | 1 μM |
| IWR-1-endo | 2.5 μM |
| WH-4-023 | 1 μM |
| Recombinant human Activin A | 25 ng/mL |
| Recombinant human FGF2 | 10 ng/mL |
| Recombinant human LIF | 10 ng/mL |

It is noted that the concentrations of the first component, the second component, the third component, the fourth component, the fifth component, and the sixth component described above all refer to the final concentration of each component in the culture medium.

In some embodiments, the culture medium further comprises a seventh component, which is a ROCK inhibitor. Addition of a ROCK inhibitor such as Y-27632 promotes proliferation of pluripotent stem cells.

In some embodiments, the seventh component is Y-27632.

In some embodiments, the concentration of the seventh component is 0.01-50 μM, for example 0.01 μM, 0.05 μM, 0.1 μM, 0.3 μM, 0.5 μM, 0.7 μM, 0.9 μM, 1 μM, 1.1 μM, 1.3 μM, 1.5 μM, 1.7 μM, 1.9 μM, 2.0 μM, 2.1 μM, 2.3 μM, 2.5 μM, 2.7 μM, 2.9 μM, 3.0 μM, 3.1 μM, 3.3 μM, 3.5 μM, 3.7 μM, 3.9 μM, 4.0 μM, 4.1 μM, 4.3 μM, 4.5 μM, 4.7 μM, 4.9 μM, 5.0 μM, 5.1 μM, 5.3 μM, 5.5 μM, 5.7 μM, 5.9 μM, 6.0 μM, 6.1 μM, 6.3 μM, 6.5 μM, 6.7 μM, 6.9 μM, 7.0 μM, 7.1 μM, 7.3 μM, 7.5 μM, 7.7 μM, 7.9 μM, 8.0 μM, 8.1 μM, 8.3 μM, 8.5 μM, 8.7 μM, 8.9 μM, 9.0 μM, 9.1 μM, 9.3 μM, 9.5 μM, 9.7 μM, 9.9 μM, 10 μM, 10.1 μM, 10.3 μM, 10.5 μM, 10.7 μM, 10.9 μM, 11 μM, 11.1 μM, 11.3 μM, 11.5 μM, 11.7 μM, 11.9 μM, 12 μM, 12.1 μM, 12.3 μM, 12.5 μM, 12.7 μM, 12.9 μM, 13 μM, 13.1 μM, 13.3 μM, 13.5 μM, 13.7 μM, 13.9 μM, 14 μM, 14.1 μM, 14.3 μM, 14.5 μM, 14.7 μM, 14.9 μM, 15 μM, 15.5 μM, 16 μM, 16.5 μM, 17 μM, 17.5 μM, 18 μM, 18.5 μM, 19 μM, 19.5 μM, 20 μM, 20.5 μM, 21 μM, 21.5 μM, 22 μM, 22.5 μM, 23 μM, 23.5 μM, 24 μM, 24.5 μM, 25 μM, 25.5 μM, 26 μM, 26.5 μM, 27 μM, 27.5 μM, 28 μM, 28.5 μM, 29 μM, 29.5 μM, 30 μM, 30.5 μM, 31 μM, 31.5 μM, 32 μM, 32.5 μM, 33 μM, 33.5 μM, 34 μM, 34.5 μM, 35 μM, 35.5 μM, 36 μM, 36.5 μM, 37 μM, 37.5 μM, 38 μM, 38.5 μM, 39 μM, 39.5 μM, 40 μM, 40.5 μM, 41 μM, 41.5 μM, 42 μM, 42.5 μM, 43 μM, 43.5 μM, 44 μM, 44.5 μM, 45 μM, 45.5 μM, 46 μM, 46.5 μM, 47 μM, 47.5 μM, 48 μM, 48.5 μM, 49 μM, 49.5 μm or 50 μM, alternatively, 0.01-0.05 μM, 0.05-0.1 μM, 0.1-0.5 μM, 0.5-1 μM, 1-1.5 μM, 1.5-2 μM, 2-2.5 μM, 2.5-3 μM, 3-3.5 μM, 3.5-4 μM, 4-4.5 μM, 4.5-5 μM, 5-5.5 μM, 5.5-6 μM, 6-6.5 μM, 6.5-7 μM, 7-7.5 μM, 7.5-8 μM, 8-8.5 μM, 8.5-9 μM, 9-9.5 μM, 9.5-10 μM, 10-10.5 μM, 10.5-11 μM, 11-11.5 μM, 11.5-12 μM, 12-12.5 μM, 12.5-13 μM, 13-13.5 μM, 13.5-14 μM, 14-14.5 μM, 14.5-15 μM, 15-20 μM, 20-23 μM, 23-25 μM, 25-27 μM, 27-30 μM, 30-33 μM, 33-35 μM, 35-37 μM, 37-40 μM, 40-43 μM, 43-45 μM, 45-47 μM or 47-50 μM.

It is noted that the concentration of the seventh component refers to the final concentration of the component in the culture medium.

In some embodiments, the culture medium further comprises an eighth component, which is a basal medium.

In some embodiments, the basal medium is a basal medium for culturing mammalian (preferably porcine) pluripotent stem cells.

In some embodiments, the basal medium comprises minimal medium, N2 supplement, B27 supplement, non-essential amino acids, beta-mercaptoethanol, knockout serum replacement, and any one selected from GlutaMAX and glutamine.

In some embodiments, the basal medium comprises minimal medium, N2 supplement, B27 supplement, non-essential amino acids, β-mercaptoethanol, knockout serum replacement and GlutaMAX.

In some embodiments, the basal medium comprises minimal medium, N2 supplement, B27 supplement, non-essential amino acids, β-mercaptoethanol, knockout serum replacement, ascorbic acid, GlutaMAX and penicillin-streptomycin.

In some embodiments, the minimal medium is selected from DMEM/F12, Neurobasal, DMEM, KO-DMEM, RPMI1640, MEM, mTeSR1 or any combination thereof.

In some embodiments, the minimal medium is selected from DMEM/F12, Neurobasal or a combination thereof.

In some embodiments, the minimal medium is DMEM/F12 and Neurobasal.

In some embodiments, the minimal medium has a volume fraction of 1%-99%, such as 1%, 3%, 5%, 7%, 9%, 10%, 11%, 13%, 15%, 17%, 19%, 20%, 21%, 23%, 25%, 27%, 29%, 30%, 31%, 33%, 35%, 37%, 39%, 40%, 41%, 42%, 43%, 44%, 45%, 45.5%, 46%, 47%, 48%, 49%, 50%, 51%, 53%, 55%, 57%, 59%, 60%, 61%, 63%, 65%, 67%, 69%, 70%, 71%, 73%, 75%, 77%, 79%, 80%, 81%, 83%, 85%, 87%, 89%, 90%, 91%, 93%, 95%, 97% or 99%, alternatively, for example, 1%-3%, 3%-5%, 5%-7%, 7%-9%, 9%-11%, 11%-13%, 13%-15%, 15%-17%, 17%-19%, 19%-20%, 20%-21%, 21%-23%, 23%-25%, 25%-27%, 27%-29%, 29%-30%, 30%-31%, 31%-33%, 33%-35%, 35%-37%, 37%-39%, 39%-40%, 40%-41%, 41%-42%, 42%-43%, 43%-44%, 44%-45%, 45%-45.5%, 45.5%-46%, 46%-47%, 47%-48%, 48%-49%, 49%-50%, 50%-51%, 51%-53%, 53%-55%, 55%-57%, 57%-59%, 59%-60%, 60%-61%, 61%-63%, 63%-65%, 65%-67%, 67%-69%, 69%-70%, 70%-71%, 71%-73%, 73%-75%, 75%-77%, 77%-79%, 79%-80%, 80%-81%, 81%-83%, 83%-85%, 85%-87%, 87%-89%, 89%-90%, 90%-91%, 91%-93%, 93%-95%, 95%-97% or 97%-99%.

In some embodiments, the minimal medium has a volume fraction of 91%.

In some embodiments, the volume fraction of DMEM/F12 is 1%-99%, such as 1%, 3%, 5%, 7%, 9%, 10%, 11%, 13%, 15%, 17%, 19%, 20%, 21%, 23%, 25%, 27%, 29%, 30%, 31%, 33%, 35%, 37%, 39%, 40%, 41%, 42%, 43%, 44%, 45%, 45.5%, 46%, 47%, 48%, 49%, 50%, 51%, 53%, 55%, 57%, 59%, 60%, 61%, 63%, 65%, 67%, 69%, 70%, 71%, 73%, 75%, 77%, 79%, 80%, 81%, 83%, 85%, 87%, 89%, 90%, 91%, 93%, 95%, 97% or 99%, alternatively, for example, 1%-3%, 3%-5%, 5%-7%, 7%-9%, 9%-11%, 11%-13%, 13%-15%, 15%-17%, 17%-19%, 19%-20%, 20%-21%, 21%-23%, 23%-25%, 25%-27%, 27%-29%, 29%-30%, 30%-31%, 31%-33%, 33%-35%, 35%-37%, 37%-39%, 39%-40%, 40%-41%, 41%-42%, 42%-43%, 43%-44%, 44%-45%, 45%-45.5%, 45.5%-46%, 46%-47%, 47%-48%, 48%-49%, 49%-50%, 50%-51%, 51%-53%, 53%-55%, 55%-57%, 57%-59%, 59%-60%, 60%-61%, 61%-63%, 63%-65%, 65%-67%, 67%-69%, 69%-70%, 70%-71%, 71%-73%, 73%-75%, 75%-77%, 77%-79%, 79%-80%, 80%-81%, 81%-83%, 83%-85%, 85%-87%, 87%-89%, 89%-90%, 90%-91%, 91%-93%, 93%-95%, 95%-97% or 97%-99%.

In some embodiments, the volume fraction of the minimum medium is 91%.

In some embodiments, the volume fraction of DMEM/F12 is 1%-99%, such as 1%, 3%, 5%, 7%, 9%, 10%, 11%, 13%, 15%, 17%, 19%, 20%, 21%, 23%, 25%, 27%, 29%, 30%, 31%, 33%, 35%, 37%, 39%, 40%, 41%, 42%, 43%, 44%, 45%, 45.5%, 46%, 47%, 48%, 49%, 50%, 51%, 53%, 55%, 57%, 59%, 60%, 61%, 63%, 65%, 67%, 69%, 70%, 71%, 73%, 75%, 77%, 79%, 80%, 81%, 83%, 85%, 87%, 89%, 90%, 91%, 93%, 95%, 97% or 99%, alternatively, for example, 1%-3%, 3%-5%, 5%-7%, 7%-9%, 9%-11%, 11%-13%, 13%-15%, 15%-17%, 17%-19%, 19%-20%, 20%-21%, 21%-23%, 23%-25%, 25%-27%, 27%-29%, 29%-30%, 30%-31%, 31%-33%, 33%-35%, 35%-37%, 37%-39%, 39%-40%, 40%-41%, 41%-42%, 42%-43%, 43%-44%, 44%-45%, 45%-45.5%, 45.5%-46%, 46%-47%, 47%-48%, 48%-49%, 49%-50%, 50%-51%, 51%-53%, 53%-55%, 55%-57%, 57%-59%, 59%-60%, 60%-61%, 61%-63%, 63%-65%, 65%-67%, 67%-69%, 69%-70%, 70%-71%, 71%-73%, 73%-75%, 75%-77%, 77%-79%, 79%-80%, 80%-81%, 81%-83%, 83%-85%, 85%-87%, 87%-89%, 89%-90%, 90%-91%, 91%-93%, 93%-95%, 95%-97% or 97%-99%.

In some embodiments, the volume fraction of DMEM/F12 is 45%-50% (e.g., 45.5%, 46% and 46.5%).

In some embodiments, the Neurobasal has a volume fraction of 1%-99%, such as 1%, 3%, 5%, 7%, 9%, 10%, 11%, 13%, 15%, 17%, 19%, 20%, 21%, 23%, 25%, 27%, 29%, 30%, 31%, 33%, 35%, 37%, 39%, 40%, 41%, 42%, 43%, 44%, 45%, 45.5%, 46%, 47%, 48%, 49%, 50%, 51%, 53%, 55%, 57%, 59%, 60%, 61%, 63%, 65%, 67%, 69%, 70%, 71%, 73%, 75%, 77%, 79%, 80%, 81%, 83%, 85%, 87%, 89%, 90%, 91%, 93%, 95%, 97% or 99%, alternatively, for example, 1%-3%, 3%-5%, 5%-7%, 7%-9%, 9%-11%, 11%-13%, 13%-15%, 15%-17%, 17%-19%, 19%-20%, 20%-21%, 21%-23%, 23%-25%, 25%-27%, 27%-29%, 29%-30%, 30%-31%, 31%-33%, 33%-35%, 35%-37%, 37%-39%, 39%-40%, 40%-41%, 41%-42%, 42%-43%, 43%-44%, 44%-45%, 45%-45.5%, 45.5%-46%, 46%-47%, 47%-48%, 48%-49%, 49%-50%, 50%-51%, 51%-53%, 53%-55%, 55%-57%, 57%-59%, 59%-60%, 60%-61%, 61%-63%, 63%-65%, 65%-67%, 67%-69%, 69%-70%, 70%-71%, 71%-73%, 73%-75%, 75%-77%, 77%-79%, 79%-80%, 80%-81%, 81%-83%, 83%-85%, 85%-87%, 87%-89%, 89%-90%, 90%-91%, 91%-93%, 93%-95%, 95%-97% or 97%-99%.

In some embodiments, the Neurobasal has a volume fraction of 45%-50% (e.g., 45.5%, 46% and 46.5%).

In some embodiments, the N2 supplement has a volume fraction of 0.002%-10%, such as 0.002%, 0.005%, 0.01%, 0.015%, 0.02%, 0.025%, 0.03%, 0.035%, 0.04%, 0.045%, 0.05%, 0.055%, 0.06%, 0.065%, 0.07%, 0.075%, 0.08%, 0.085%, 0.09%, 0.095%, 0.1%, 0.13%, 0.15%, 0.17%, 0.2%, 0.25%, 0.3%, 0.35%, 0.4%, 0.45%, 0.5%, 0.55%, 0.6%, 0.65%, 0.7%, 0.75%, 0.8%, 0.85%, 0.9%, 0.95%, 1%, 1.1%, 1.3%, 1.5%, 1.7%, 1.9%, 2.0%, 2.1%, 2.3%, 2.5%, 2.7%, 2.9%, 3.0%, 3.1%, 3.3%, 3.5%, 3.7%, 3.9%, 4.0%, 4.1%, 4.3%, 4.5 4.5%, 4.7%, 4.9%, 5.0%, 5.1%, 5.3%, 5.5%, 5.7%, 5.9%, 6.0%, 6.1%, 6.3%, 6.5%, 6.7%, 6.9%, 7.0%, 7.1%, 7.3%, 7.5%, 7.7%, 7.9%, 8.0%, 8.1%, 8.3%, 8.5%, 8.7%, 8.9%, 9.0%, 9.1%, 9.3%, 9.5%, 9.7%, 9.9% or 10%, alternatively, 0.002%-0.05%, 0.05%-0.1%, 0.1%-0.15%, 0.15%-0.2%, 0.2%-0.25%, 0.25%-0.3%, 0.3%-0.35%, 0.35%-0.4%, 0.4%-0.45%, 0.45%-0.5%, 0.5%-0.55%, 0.55%-0.6%, 0.6%-0.65%, 0.65%-0.7%, 0.7%-0.75%, 0.75%-0.8%, 0.8%-0.85%, 0.85%-0.9%, 0.9%-0.95%, 0.95%-1.0%, 1.0%-1.1%, 1.1%-1.3%, 1.3%-1.5%, 1.5%-1.7%, 1.7%-1.9%, 1.9%-2.0%, 2.0%-2.1%, 2.1%-2.3%, 2.3%-2.5%, 2.5%-2.7%, 2.7%-2.9%, 2.9%-3.0%, 3.0%-3.1%, 3.1%-3.3%, 3.3%-3.5%, 3.5%-3.7%, 3.7%-3.9%, 3.9%-4.0%, 4.0%-4.1%, 4.1%-4.3%, 4.3%-4.5%, 4.5%-4.7%, 4.7%-4.9%, 4.9%-5.0%, 5.0%-5.5%, 5.5%-6.0%, 6.0%-6.5%, 6.5%-7.0%, 7.0%-7.5%, 7.5%-8.0%, 8.0%-8.5%, 8.5%-9.0%, 9.0%-9.5% or 9.5%-10%.

In some embodiments, the N2 supplement has a volume fraction of 0.5%.

In some embodiments, the B27 supplement has a volume fraction of 0.002%-20%, such as 0.002%, 0.005%, 0.01%, 0.015%, 0.02%, 0.025%, 0.03%, 0.035%, 0.04%, 0.045%, 0.05%, 0.055%, 0.06%, 0.065%, 0.07%, 0.075%, 0.08%, 0.085%, 0.09%, 0.095%, 0.1%, 0.13%, 0.15%, 0.17%, 0.2%, 0.25%, 0.3%, 0.35%, 0.4%, 0.45%, 0.5%, 0.55%, 0.6%, 0.65%, 0.7%, 0.75%, 0.8%, 0.85%, 0.9%, 0.95%, 1%, 1.1%, 1.3%, 1.5%, 1.7%, 1.9%, 2.0%, 2.1%, 2.3%, 2.5%, 2.7%, 2.9%, 3.0%, 3.1%, 3.3%, 3.5%, 3.7%, 3.9%, 4.0%, 4.1%, 4.3%, 4.5%, 4.7%, 4.9%, 5.0%, 5.1%, 5.3%, 5.5%, 5.7%, 5.9%, 6.0%, 6.1%, 6.3%, 6.5%, 6.7%, 6.9%, 7.0%, 7.1%, 7.3%, 7.5%, 7.7%, 7.9%, 8.0%, 8.1%, 8.3%, 8.5%, 8.7%, 8.9%, 9.0%, 9.1%, 9.3%, 9.5%, 9.7%, 9.9%, 10%, 10.1%, 10.3%, 10.5%, 10.7%, 10.9%, 11%, 11.1%, 11.3%, 11.5%, 11.7%, 11.9%, 12%, 12.1%, 12.3%, 12.5%, 12.7%, 12.9%, 13%, 13.1%, 13.3%, 13.5%, 13.7%, 13.9%, 14%, 14.1%, 14.3%, 14.5%, 14.7%, 14.9%, 15%, 15.5%, 16%, 16.5%, 17%, 17.5%, 18%, 18.5%, 19%, 19.5% or 20%, alternatively, 0.002%-0.05%, 0.05%-0.1%, 0.1%-0.15%, 0.15%-0.2%, 0.2%-0.25%, 0.25%-0.3%, 0.3%-0.35%, 0.35%-0.4%, 0.4%-0.45%, 0.45%-0.5%, 0.5%-0.55%, 0.55%-0.6%, 0.6%-0.65%, 0.65%-0.7%, 0.7%-0.75%, 0.75%-0.8%, 0.8%-0.85%, 0.85%-0.9%, 0.9%-0.95%, 0.95%-1.0%, 1.0%-1.1%, 1.1%-1.3%, 1.3%-1.5%, 1.5%-1.7%, 1.7%-1.9%, 1.9%-2.0%, 2.0%-2.1%, 2.1%-2.3%, 2.3%-2.5%, 2.5%-2.7%, 2.7%-2.9%, 2.9%-3.0%, 3.0%-3.1%, 3.1%-3.3%, 3.3%-3.5%, 3.5%-3.7%, 3.7%-3.9%, 3.9%-4.0%, 4.0%-4.1%, 4.1%-4.3%, 4.3%-4.5%, 4.5%-4.7%, 4.7%-4.9%, 4.9%-5.0%, 5.0%-5.5%, 5.5%-6.0%, 6.0%-6.5%, 6.5%-7.0%, 7.0%-7.5%, 7.5%-8.0%, 8.0%-8.5%, 8.5%-9.0%, 9.0%-9.5%, 9.5%-10%, 10%-10.5%, 10.5%-11%, 11%-11.5%, 11.5%-12%, 12%-12.5%, 12.5%-13%, 13%-13.5%, 13.5%-14%, 14%-14.5%, 14.5%-15% or 15%-20%.

In some embodiments, the B27 supplement has a volume fraction of 1%.

In some embodiments, the non-essential amino acid has a volume fraction of 0.01%-10%, such as 0.01%, 0.05%, 0.1%, 0.2%, 0.25%, 0.3%, 0.35%, 0.4%, 0.45%, 0.5%, 0.55%, 0.6%, 0.65%, 0.7%, 0.75%, 0.8%, 0.85%, 0.9%, 0.95%, 1%, 1.1%, 1.3%, 1.5%, 1.7%, 1.9%, 2.0%, 2.1%, 2.3%, 2.5%, 2.7%, 2.9%, 3.0%, 3.1%, 3.3%, 3.5%, 3.7%, 3.9%, 4.0%, 4.1%, 4.3%, 4.5%, 4.7%, 4.9%, 5.0%, 5.1%, 5.3%, 5.5%, 5.7%, 5.9%, 6.0%, 6.1%, 6.3%, 6.5%, 6.7%, 6.9%, 7.0%, 7.1%, 7.3%, 7.5%, 7.7%, 7.9%, 8.0%, 8.1%, 8.3%, 8.5%, 8.7%, 8.9%, 9.0%, 9.1%, 9.3%, 9.5%, 9.7%, 9.9% or 10%, alternatively, 0.01%-0.05%, 0.05%-0.1%, 0.1%-0.2%, 0.2%-0.25%, 0.25%-0.3%, 0.3%-0.35%, 0.35%-0.4%, 0.4%-0.45%, 0.45%-0.5%, 0.5%-0.55%, 0.55%-0.6%, 0.6%-0.65%, 0.65%-0.7%, 0.7%-0.75%, 0.75%-0.8%, 0.8%-0.85%, 0.85%-0.9%, 0.9%-0.95%, 0.95%-1.0%, 1.0%-1.1%, 1.1%-1.3%, 1.3%-1.5%, 1.5%-1.7%, 1.7%-1.9%, 1.9%-2.0%, 2.0%-2.1%, 2.1%-2.3%, 2.3%-2.5%, 2.5%-2.7%, 2.7%-2.9%, 2.9%-3.0%, 3.0%-3.1%, 3.1%-3.3%, 3.3%-3.5%, 3.5%-3.7%, 3.7%-3.9%, 3.9%-4.0%, 4.0%-4.1%, 4.1%-4.3%, 4.3%-4.5%, 4.5%-4.7%, 4.7%-4.9%, 4.9%-5.0%, 5.0%-5.5%, 5.5%-6.0%, 6.0%-6.5%, 6.5%-7.0%, 7.0%-7.5%, 7.5%-8.0%, 8.0%-8.5%, 8.5%-9.0%, 9.0%-9.5% or 9.5%-10%.

In some embodiments, the non-essential amino acid has a volume fraction of 1%.

In some embodiments, the concentration of the β-mercaptoethanol is 0.01 mM-1 mM, e.g., 0.01 mM, 0.02 mM, 0.03 mM, 0.04 mM, 0.05 mM, 0.06 mM, 0.07 mM, 0.08 mM, 0.09 mM, 0.1 mM, 0.11 mM, 0.12 mM, 0.13 mM, 0.14 mM, 0.15 mM, 0.16 mM, 0.17 mM, 0.18 mM, 0.19 mM, 0.2 mM, 0.21 mM, 0.22 mM, 0.23 mM, 0.24 mM, 0.25 mM, 0.26 mM, 0.27 mM, 0.28 mM, 0.29 mM, 0.3 mM, 0.31 mM, 0.32 mM, 0.33 mM, 0.34 mM, 0.35 mM, 0.36 mM, 0.37 mM, 0.38 mM, 0.39 mM, 0.4 mM, 0.41 mM, 0.42 mM, 0.43 mM, 0.44 mM, 0.45 mM, 0.46 mM, 0.47 mM, 0.48 mM, 0.49 mM, 0.5 mM, 0.51 mM, 0.53 mM, 0.55 mM, 0.57 mM, 0.59 mM, 0.6 mM, 0.61 mM, 0.63 mM, 0.65 mM, 0.67 mM, 0.69 mM, 0.7 mM, 0.71 mM, 0.73 mM, 0.75 mM, 0.77 mM, 0.79 mM, 0.8 mM, 0.81 mM, 0.83 mM, 0.85 mM, 0.87 mM, 0.89 mM, 0.9 mM, 0.91 mM, 0.93 mM, 0.95 mM, 0.97 mM, 0.99 mM or 1 mM, alternatively, 0.01-0.02 mM, 0.02-0.03 mM, 0.03-0.04 mM, 0.04-0.05 mM, 0.05-0.06 mM, 0.06-0.07 mM, 0.07-0.08 mM, 0.08-0.09 mM, 0.09-0.1 mM, 0.1-0.11 mM, 0.11-0.12 mM, 0.12-0.13 mM, 0.13-0.14 mM, 0.14-0.15 mM, 0.15-0.16 mM, 0.16-0.17 mM, 0.17-0.18 mM, 0.18-0.19 mM, 0.19-0.2 mM, 0.2-0.21 mM, 0.21-0.23 mM, 0.23-0.25 mM, 0.25-0.27 mM, 0.27-0.29 mM, 0.29-0.3 mM, 0.3-0.31 mM, 0.31-0.33 mM, 0.33-0.35 mM, 0.35-0.37 mM, 0.37-0.39 mM, 0.39-0.4 mM, 0.4-0.41 mM, 0.41-0.43 mM, 0.43-0.45 mM, 0.45-0.47 mM, 0.47-0.49 mM, 0.49-0.5 mM, 0.5-0.55 mM, 0.55-0.6 mM, 0.6-0.65 mM, 0.65-0.7 mM, 0.7-0.75 mM, 0.75-0.8 mM, 0.8-0.85 mM, 0.85-0.9 mM, 0.9-0.95 mM or 0.95-1 mM.

In some embodiments, the concentration of the β-mercaptoethanol is 0.1 mM.

In some embodiments, the knockout serum replacement has a volume fraction of 0.01%-50%, such as 0.01%, 0.05%, 0.1%, 0.2%, 0.25%, 0.3%, 0.35%, 0.4%, 0.45%, 0.5%, 0.55%, 0.6%, 0.65%, 0.7%, 0.75%, 0.8%, 0.85%, 0.9%, 0.95%, 1%, 1.1%, 1.3%, 1.5%, 1.7%, 1.9%, 2.0%, 2.1%, 2.3%, 2.5%, 2.7%, 2.9%, 3.0%, 3.1%, 3.3%, 3.5%, 3.7%, 3.9%, 4.0%, 4.1%, 4.3%, 4.5%, 4.7%, 4.9%, 5.0%, 5.1%, 5.3%, 5.5%, 5.7%, 5.9%, 6.0%, 6.1%, 6.3%, 6.5%, 6.7%, 6.9%, 7.0%, 7.1%, 7.3%, 7.5%, 7.7%, 7.9%, 8.0%, 8.1%, 8.3%, 8.5%, 8.7%, 8.9%, 9.0%, 9.1%, 9.3%, 9.5%, 9.7%, 9.9%, 10%, 10.1%, 10.3%, 10.5%, 10.7%, 10.9%, 11%, 11.1%, 11.3%, 11.5%, 11.7%, 11.9%, 12%, 12.1%, 12.3%, 12.5%, 12.7%, 12.9%, 13%, 13.1%, 13.3%, 13.5%, 13.7%, 13.9%, 14%, 14.1%, 14.3%, 14.5%, 14.7%, 14.9%, 15%, 15.5%, 16%, 16.5%, 17%, 17.5%, 18%, 18.5%, 19%, 19.5%, 20%, 20.5%, 21%, 21.5%, 22%, 22.5%, 23%, 23.5%, 24%, 24.5%, 25%, 25.5%, 26%, 26.5%, 27%, 27.5%, 28%, 28.5%, 29%, 29.5%, 30%, 35%, 40%, 45% or 50%, alternatively, 0.01%-0.05%, 0.05%-0.1%, 0.1%-0.2%, 0.2%-0.25%, 0.25%-0.3%, 0.3%-0.35%, 0.35%-0.4%, 0.4%-0.45%, 0.45%-0.5%, 0.5%-0.55%, 0.55%-0.6%, 0.6%-0.65%, 0.65%-0.7%, 0.7%-0.75%, 0.75%-0.8%, 0.8%-0.85%, 0.85%-0.9%, 0.9%-0.95%, 0.95%-1.0%, 1.0%-1.1%, 1.1%-1.3%, 1.3%-1.5%, 1.5%-1.7%, 1.7%-1.9%, 1.9%-2.0%, 2.0%-2.1%, 2.1%-2.3%, 2.3%-2.5%, 2.5%-2.7%, 2.7%-2.9%, 2.9%-3.0%, 3.0%-3.1%, 3.1%-3.3%, 3.3%-3.5%, 3.5%-3.7%, 3.7%-3.9%, 3.9%-4.0%, 4.0%-4.1%, 4.1%-4.3%, 4.3%-4.5%, 4.5%-4.7%, 4.7%-4.9%, 4.9%-5.0%, 5.0%-5.5%, 5.5%-6.0%, 6.0%-6.5%, 6.5%-7.0%, 7.0%-7.5%, 7.5%-8.0%, 8.0%-8.5%, 8.5%-9.0%, 9.0%-9.5%, 9.5%-10%, 10%-10.5%, 10.5%-11%, 11%-11.5%, 11.5%-12%, 12%-12.5%, 12.5%-13%, 13%-13.5%, 13.5%-14%, 14%-14.5%, 14.5%-15%, 15%-20%, 20%-25%, 25%-30%, 30%-35%, 35%-40%, 40%-45% or 45%-50%.

In some embodiments, the knockout serum replacement has a volume fraction of 5%.

In some embodiments, the concentration of the ascorbic acid is 1 μg/mL-5000 μg/mL, such as 1 μg/mL, 5 μg/mL, 10 μg/mL, 15 μg/mL, 20 μg/mL, 25 μg/mL, 30 μg/mL, 35 μg/mL, 40 μg/mL, 41 μg/mL, 42 μg/mL, 43 μg/mL, 44 μg/mL, 45 μg/mL, 46 μg/mL, 47 μg/mL, 48 μg/mL, 49 μg/mL, 50 μg/mL, 51 μg/mL, 52 μg/mL, 53 μg/mL, 54 μg/mL, 55 μg/mL, 56 μg/mL, 57 μg/mL, 58 μg/mL, 59 μg/mL, 60 μg/mL, 65 μg/mL, 70 μg/mL, 75 μg/mL, 80 μg/mL, 85 μg/mL, 90 μg/mL, 95 μg/mL, 100 μg/mL, 110 μg/mL, 120 μg/mL, 130 μg/mL, 140 μg/mL, 150 μg/mL, 160 μg/mL, 170 μg/mL, 180 μg/mL, 190 μg/mL, 200 μg/mL, 210 μg/mL, 220 μg/mL, 230 μg/mL, 240 μg/mL, 250 μg/mL, 260 μg/mL, 270 μg/mL, 280 μg/mL, 290 μg/mL, 300 μg/mL, 310 μg/mL, 320 μg/mL, 330 μg/mL, 340 μg/mL, 350 μg/mL, 360 μg/mL, 370 μg/mL, 380 μg/mL, 390 μg/mL, 400 μg/mL, 410 μg/mL, 420 μg/mL, 430 μg/mL, 440 μg/mL, 450 μg/mL, 460 μg/mL, 470 μg/mL, 480 μg/mL, 490 μg/mL, 500 μg/mL, 550 μg/mL, 600 μg/mL, 650 μg/mL, 700 μg/mL, 750 μg/mL, 800 μg/mL, 850 μg/mL, 900 μg/mL, 950 μg/mL, 1000 μg/mL, 2000 μg/mL, 3000 μg/mL, 4000 μg/mL or 5000 μg/mL, alternatively, 1-5 μg/mL, 5-10 μg/mL, 10-15 μg/mL, 15-20 μg/mL, 20-25 μg/mL, 25-30 μg/mL, 30-35 μg/mL, 35-40 μg/mL, 40-45 μg/mL, 45-50 μg/mL, 50-55 μg/mL, 55-60 μg/mL, 60-65 μg/mL, 65-70 μg/mL, 70-75 μg/mL, 75-80 μg/mL, 80-85 μg/mL, 85-90 μg/mL, 90-95 μg/mL, 95-100 μg/mL, 100-110 μg/mL, 110-120 μg/mL, 120-130 μg/mL, 130-140 μg/mL, 140-150 μg/mL, 150-170 μg/mL, 170-190 μg/mL, 190-200 μg/mL, 200-210 μg/mL, 210-230 μg/mL, 230-250 μg/mL, 250-270 μg/mL, 270-290 μg/mL, 290-300 μg/mL, 300-310 μg/mL, 310-330 μg/mL, 330-350 g/mL, 350-370 μg/mL, 370-390 μg/mL, 390-400 μg/mL, 400-410 μg/mL, 410-430 μg/mL, 430-450 μg/mL, 450-470 μg/mL, 470-490 μg/mL, 490-500 μg/mL, 500-550 μg/mL, 550-600 μg/mL, 600-650 μg/mL, 650-700 μg/mL, 700-750 μg/mL, 750-800 μg/mL, 800-850 μg/mL, 850-950 μg/mL, 900-950 μg/mL, 950-1000 μg/mL, 1000-2000 μg/mL, 2000-3000 μg/mL, 3000-4000 μg/mL or 4000-5000 μg/mL.

In some embodiments, the concentration of the ascorbic acid is 50 μg/mL.

In some embodiments, the GlutaMAX or glutamine (preferably GlutaMAX) has a volume fraction of 0.01%-10%, such as 0.01%, 0.1%, 0.2%, 0.25%, 0.3%, 0.35%, 0.4%, 0.45%, 0.5%, 0.55%, 0.6%, 0.65%, 0.7%, 0.75%, 0.8%, 0.85%, 0.9%, 0.95%, 1%, 1.1%, 1.3%, 1.5%, 1.7%, 1.9%, 2.0%, 2.1%, 2.3%, 2.5%, 2.7%, 2.9%, 3.0%, 3.1%, 3.3%, 3.5%, 3.7%, 3.9%, 4.0%, 4.1%, 4.3%, 4.5%, 4.7%, 4.9%, 5.0%, 5.1%, 5.3%, 5.5%, 5.7%, 5.9%, 6.0%, 6.1%, 6.3%, 6.5%, 6.7%, 6.9%, 7.0%, 7.1%, 7.3%, 7.5%, 7.7%, 7.9%, 8.0%, 8.1%, 8.3%, 8.5%, 8.7%, 8.9%, 9.0%, 9.1%, 9.3%, 9.5%, 9.7%, 9.9% or 10%, alternatively, 0.01%-0.1%, 0.1%-0.2%, 0.2%-0.25%, 0.25%-0.3%, 0.3%-0.35%, 0.35%-0.4%, 0.4%-0.45%, 0.45%-0.5%, 0.5%-0.55%, 0.55%-0.6%, 0.6%-0.65%, 0.65%-0.7%, 0.7%-0.75%, 0.75%-0.8%, 0.8%-0.85%, 0.85%-0.9%, 0.9%-0.95%, 0.95%-1.0%, 1.0%-1.1%, 1.1%-1.3%, 1.3%-1.5%, 1.5%-1.7%, 1.7%-1.9%, 1.9%-2.0%, 2.0%-2.1%, 2.1%-2.3%, 2.3%-2.5%, 2.5%-2.7%, 2.7%-2.9%, 2.9%-3.0%, 3.0%-3.1%, 3.1%-3.3%, 3.3%-3.5%, 3.5%-3.7%, 3.7%-3.9%, 3.9%-4.0%, 4.0%-4.1%, 4.1%-4.3%, 4.3%-4.5%, 4.5%-4.7%, 4.7%-4.9%, 4.9%-5.0%, 5.0%-5.5%, 5.5%-6.0%, 6.0%-6.5%, 6.5%-7.0%, 7.0%-7.5%, 7.5%-8.0%, 8.0%-8.5%, 8.5%-9.0%, 9.0%-9.5% or 9.5%-10%.

In some embodiments, the GlutaMAX or glutamine (preferably GlutaMAX) has a volume fraction of 0.5%.

In some embodiments, the penicillin-streptomycin has a volume fraction of 0.01%-20%, such as 0.01%, 0.1%, 0.2%, 0.25%, 0.3%, 0.35%, 0.4%, 0.45%, 0.5%, 0.55%, 0.6%, 0.65%, 0.7%, 0.75%, 0.8%, 0.85%, 0.9%, 0.95%, 1%, 1.1%, 1.3%, 1.5%, 1.7%, 1.9%, 2.0%, 2.1%, 2.3%, 2.5%, 2.7%, 2.9%, 3.0%, 3.1%, 3.3%, 3.5%, 3.7%, 3.9%, 4.0%, 4.1%, 4.3%, 4.5%, 4.7%, 4.9%, 5.0%, 5.1%, 5.3%, 5.5%, 5.7%, 5.9%, 6.0%, 6.1%, 6.3%, 6.5%, 6.7%, 6.9%, 7.0%, 7.1%, 7.3%, 7.5%, 7.7%, 7.9%, 8.0%, 8.1%, 8.3%, 8.5%, 8.7%, 8.9%, 9.0%, 9.1%, 9.3%, 9.5%, 9.7%, 9.9%, 10%, 10.1%, 10.3%, 10.5%, 10.7%, 10.9%, 11%, 11.1%, 11.3%, 11.5%, 11.7%, 11.9%, 12%, 12.1%, 12.3%, 12.5%, 12.7%, 12.9%, 13%, 13.1%, 13.3%, 13.5%, 13.7%, 13.9%, 14%, 14.1%, 14.3%, 14.5%, 14.7%, 14.9%, 15%, 15.5%, 16%, 16.5%, 17%, 17.5%, 18%, 18.5%, 19%, 19.5% or 20%, alternatively, 0.01-0.1%, 0.1%-0.2%, 0.2%-0.25%, 0.25%-0.3%, 0.3%-0.35%, 0.35%-0.4%, 0.4%-0.45%, 0.45%-0.5%, 0.5%-0.55%, 0.55%-0.6%, 0.6%-0.65%, 0.65%-0.7%, 0.7%-0.75%, 0.75%-0.8%, 0.8%-0.85%, 0.85%-0.9%, 0.9%-0.95%, 0.95%-1.0%, 1.0%-1.1%, 1.1%-1.3%, 1.3%-1.5%, 1.5%-1.7%, 1.7%-1.9%, 1.9%-2.0%, 2.0%-2.1%, 2.1%-2.3%, 2.3%-2.5%, 2.5%-2.7%, 2.7%-2.9%, 2.9%-3.0%, 3.0%-3.1%, 3.1%-3.3%, 3.3%-3.5%, 3.5%-3.7%, 3.7%-3.9%, 3.9%-4.0%, 4.0%-4.1%, 4.1%-4.3%, 4.3%-4.5%, 4.5%-4.7%, 4.7%-4.9%, 4.9%-5.0%, 5.0%-5.5%, 5.5%-6.0%, 6.0%-6.5%, 6.5%-7.0%, 7.0%-7.5%, 7.5%-8.0%, 8.0%-8.5%, 8.5%-9.0%, 9.0%-9.5%, 9.5%-10%, 10%-10.5%, 10.5%-11%, 11%-11.5%, 11.5%-12%, 12%-12.5%, 12.5%-13%, 13%-13.5%, 13.5%-14%, 14%-14.5%, 14.5%-15% or 15%-20%. The addition of the penicillin-streptomycin facilitates the prevention of cells from being infected.

In some embodiments, the penicillin-streptomycin has a volume fraction of 1%.

In some embodiments, the volume ratio of the DMEM/F12 to the Neurobasal is 5:1-1:5, such as 5:1, 4:1, 3:1, 2:1, 1:1, 1:2, 1:3, 1:4 or 1:5, alternatively, 5:1-4:1, 4:1-3:1, 3:1-2:1, 2:1-1:1, 1:1-1:2, 1:2-1:3, 1:3-1:4 or 1:4-1:5.

In some embodiments, the volume ratio of the DMEM/F12 to the Neurobasal is 1:1.

It should be noted that the concentration of each specific ingredient in the above-described eighth component refers to the final concentration of each specific ingredient in the culture medium. In addition, the volume fraction of each specific ingredient in the above-described eighth component refers to the volume of the specific ingredient/total volume of the culture medium.

In some embodiments included per 500 mL of the culture medium.

| Name | Concentration/volume/volume fraction |
|---|---|
| CHIR99021 | 1 μM |
| IWR-1-endo | 2.5 μM |
| WH-4-023 | 1 μM |
| Recombinant human Activin A | 25 ng/mL |
| Recombinant human FGF2 | 10 ng/mL |
| Recombinant human LIF | 10 ng/mL |
| DMEM/F12 | 227.5 mL |
| Neurobasal | 227.5 mL |
| N2 supplement | 2.5 mL |
| B27 supplement | 5 mL |
| GlutaMAX | volume fraction: 0.5% |
| Non-essential amino acid | volume fraction: 1% |
| β-Mercaptoethanol | 0.1 mM |
| Penicillin-streptomycin | volume fraction: 1% |
| Knockout serum replacement | volume fraction: 5% |
| Ascorbic acid | 50 μg/mL |
| Y-27632(optional) | 10 μM or 2 μM |

It should also be noted that each of the component in the culture medium or the specific ingredients in each component mentioned above are reagents commonly used by those skilled in the art and are commercially available.

In some embodiments, commercially available examples are as follows:

| Name | Brand and Catalogue number |
|---|---|
| DMEM/F12 | Thermo Fisher 729 Scientific, 10565-018 |
| Neurobasal | Thermo Fisher Scientific, 21103-049 |
| N2 supplement | Thermo Fisher Scientific, 17502-048 |
| B27 supplement | Thermo Fisher Scientific, 12587-010 |
| GlutaMAX | Thermo Fisher Scientific, 35050-061 |
| Non-essential amino acid | Thermo Fisher 732 Scientific, 11140-050 |
| β-Mercaptoethanol | Thermo Fisher Scientific, 21985-023 |
| Penicillin-streptomycin | Thermo FisherScientific, 15140-122 |
| Knockout serum replacement | KOSR, Thermo Fisher Scientific, |
| | A3181502, optional |
| Ascorbic acid | Sigma-Aldrich, A4544 |
| CHIR99021 | Selleckchem, S1263 |
| IWR-1-endo | Selleckchem, S7086 |
| WH-4-023 | Selleckchem, S7565 |
| Recombinant human LIF | Pepro Tech, 300-05 |
| Recombinant human Activin A | Peprotech, 120-14E |
| Recombinant human FGF2 | Peprotech, 100-18B |
| Y-27632 | Selleckchem, S1049 |

In some embodiments, the non-essential amino acid comprises glycine, L-alanine, L-asparagine, L-aspartic acid, L-glutamic acid, L-proline and L-serine.

In some embodiments, the non-essential amino acid comprises:

| Ingredients | Concentration(mg/L) | Concentration(mM) |
|---|---|---|
| glycine | 750.0 | 10.0 |
| L-alanine | 890.0 | 10.0 |
| L-asparagine | 1320.0 | 10.0 |
| L-aspartic acid | 1330.0 | 10.0 |
| L-glutamic acid | 1470.0 | 10.0 |
| L-proline | 1150.0 | 10.0 |
| L-serine | 1050.0 | 10.0 |

It should be noted that in the above table, the concentration of each ingredient of the non-essential amino acid refers to the concentration of each specific ingredient in the non-essential amino acid.

In some exemplary embodiments, the culture medium contains: a first component, which is IWR-1-endo; a second component, which is selected from WH-4-023 and A419259; a third component, which is selected from fibroblast growth factors. In some embodiments, the culture medium further contains: a fourth component, which is selected from CHIR99021 and WNT3a; a fifth component, which is selected from TGF-β superfamily members; and a sixth component, which is LIF.

In some exemplary embodiments, the culture medium has one or more of the following technical features (1) to (5):
(1) the second component is WH-4-023;
(2) the third component is selected from FGF2 and FGF1; preferably, the third component is FGF2; more preferably, the third component is recombinant human FGF2.
(3) the fourth component is CHIR99021;
(4) the fifth component is selected from Activin A and Nodal; preferably, the fifth component is Activin A; more preferably, the fifth component is recombinant human Activin A;
(5) the sixth component is selected from recombinant human LIF and recombinant mouse LIF; more preferably, the sixth group is recombinant human LIF.

In some exemplary embodiments, the culture medium has one or more of the following technical features (1) to (7):
(1) The concentration of the first component is 0.1-10 μM; preferably, the concentration of the first component is 0.9-3 μM; more preferably, the concentration of the first component is 2.5 μM;
(2) The concentration of the second component is 3 nM-30 μM; preferably, the concentration of the second component is 0.01-5 μM; more preferably, the concentration of the second component is 1 μM;
(3) The concentration of the third component is 0.01-100 ng/ml; preferably, the concentration of the third component is 1-100 ng/ml; more preferably, the concentration of the third component is 10 ng/mL;
(4) The concentration of the fourth component is 0.0025 nM-3 μM; preferably, the concentration of the fourth component is 0.01-3 μM; more preferably, the concentration of the fourth component is 1 μM;
(5) The concentration of the fifth component is 0.01-100 ng/ml; preferably, the concentration of the fifth component is 25 ng/ml;
(6) The concentration of the sixth component is 0.01-100 ng/ml; preferably, the concentration of the sixth component is 1-100 ng/ml; more preferably, the concentration of the sixth component is 10 ng/ml;
(7) The concentration ratio of the fourth component to the first component is 25:1-1:25; preferably, the concentration ratio of the fourth component to the first component is 2:3-1:3.

In some exemplary embodiments, the culture medium further contains a seventh component, which is a ROCK inhibitor; preferably, the seventh component is Y-27632; preferably, the seventh component has a concentration of 0.01-50 μM; preferably, the seventh component has a concentration of 0.01-20 μM when the culture medium is used for cell passage, preferably 10 μM; preferably, when the culture medium is used for cell maintenance, the concentration of the seventh component is 0.01-10 μM, preferably 2 μM.

In some exemplary embodiments, the medium further contains an eighth component, which is a basal medium; preferably, the basal medium is a basal medium for culturing mammalian (preferably porcine) pluripotent stem cells; more preferably, the basal medium contains minimal medium, N2 supplement, B27 supplement, non-essential amino acids, β-mercaptoethanol, knockout serum replacement and any one selected from GlutaMAX and glutamine; further preferably, the basal medium contains minimal medium, N2 supplement, B27 supplement, non-essential amino acids, β-mercaptoethanol, knockout serum replacement and GlutaMAX; most preferably, the basal medium comprises minimal medium, N2 supplement, B27 supplement, non-essential amino acids, β-mercaptoethanol, knockout serum replacement, ascorbic acid, GlutaMAX and penicillin-streptomycin; preferably, the minimal medium is selected from DMEM/F12, Neurobasal, DMEM, KO-DMEM, RPMI1640, MEM, mTeSR1 or any combination thereof; preferably, the minimal medium is selected from DMEM/F12, Neurobasal or a combination thereof; preferably, the minimal medium is selected from DMEM/F12 and Neurobasal.

In some exemplary embodiments, the basal medium has one or more of the following technical features (1) to (12):
(1) The minimal medium has a volume fraction of 1% to 99%, preferably 91%;
(2) The DMEM/F12 has a volume fraction of 1%-99%, preferably 45%-50% (e.g. 45.5%);
(3) The Neurobasal has a volume fraction of 1%-99%, preferably 45%-50% (e.g. 45.5%);
(4) The N2 supplement has a volume fraction of 0.002%-10%, preferably 0.5%;
(5) The B27 supplement has a volume fraction of 0.002%-20%, preferably 1%;
(6) The non-essential amino acids have a volume fraction of 0.01%-10%, preferably 1%;
(7) The concentration of the β-mercaptoethanol is 0.01 mM-1 mM, preferably 0.1 mM;
(8) The knockout serum replacement has a volume fraction of 0.01%-50%, preferably 5%;
(9) The concentration of the ascorbic acid is 1 μg/mL-5000 μg/mL, preferably 50 μg/mL;
(10) The volume fraction of GlutaMAX or glutamine (preferably GlutaMAX) is 0.01%-10%, preferably 0.5%;
(11) The penicillin-streptomycin has a volume fraction of 0.01%-20%, preferably 1%;
(12) The volume ratio of the DMEM/F12 to the Neurobasal is 5:1-1:5, preferably 1:1.

In some embodiments, the Epiblast derived from a pig embryo is a pig embryonic Epiblast of E8 to E10 (e.g., E8, E9, or E10).

In some embodiments, the method is performed under the condition where feeder cells are present.

In some embodiments, the feeder cells are selected from mouse embryonic fibroblasts or STO cells.

In some embodiments, the feeder cells are mouse embryonic fibroblasts.

In some embodiments, the feeder cells are mouse embryonic fibroblasts that have stopped dividing.

In some embodiments, the feeder cells are mitomycin C treated mouse embryonic fibroblasts.

In some embodiments, the feeder cells have a density of 10⁴/well˜10×10⁵/well.

In some embodiments, the feeder cells have a density of 2×10⁴/well˜2×10⁵/well.

In some embodiments, the container for culture is a 12-well culture plate.

In some embodiments, the method is performed under the condition of: temperature: 37-39° C. (preferably 37° C.), oxygen concentration: 5%-22% (preferably 5%), carbon dioxide concentration: 4%-6% (preferably 5%), and humidity: 100%.

In some embodiments, the frequency of replacement of the medium in the method is every 10-48 hours (preferably 10-24 hours, more preferably 12 hours).

In another aspect, the present invention also provides a pluripotent stem cell or cell population produced by the above-mentioned method. In some embodiments, the pluripotent stem cells are the pluripotent stem cells described in the first aspect. In some embodiments, the population of cells is a population of cells as described in the second aspect.

### Advantageous Effects of the Invention

The present invention obtains for the first time a stable pig pluripotent stem cell that expresses pluripotency markers, the pluripotent stem cell may be differentiated into 3 germ layers, has a highly plastic chromatin architecture, and tolerates multiple rounds of gene editing while retaining normal karyotypes. Using the gene-edited pig pluripotent stem cells provided by the present invention as donor cells for cell nuclear transfer, a live cloned piglet with multiple gene editing function may be successfully cloned. Therefore, the pig pluripotent stem cells of the present invention have important application value in biological research, animal husbandry and regenerative biomedicine.

Embodiments of the present invention will be described in detail below in conjunction with the accompanying drawings and examples, but it will be understood by those skilled in the art that the following accompanying drawings and examples are only used to illustrate the present invention, and do not limit the scope of the present invention. Various objects and advantageous aspects of the present invention will become apparent to those skilled in the art in light of the following detailed description of the accompanying drawings and preferred embodiments.

### BRIEF DESCRIPTION OF DRAWINGS

FIG. 1A: Morphological comparison of derivatives of embryonic epiblast and ectoderm cells at different embryonic stages. Which are, from left to right, embryonic discs (left) and outgrowths (right) of E8, E10 and E12. Epiblast cells of E8 and E10 grew spherically, and ectoderm cells of E12 grew flattened and irregularly. Scale bars for E8 and E10 embryonic discs, 100 μm; scale bar for E12 embryonic disc, 400 μm; scale bars for all outgrowths, 200 μm.

FIG. 1B: Outgrowth rates and cell lines of the cells at different embryonic stages established in 3i/LAF medium.

FIGS. 2-1A to 2-1M: Effect of different culture conditions on the preparation of pgEpiSC.

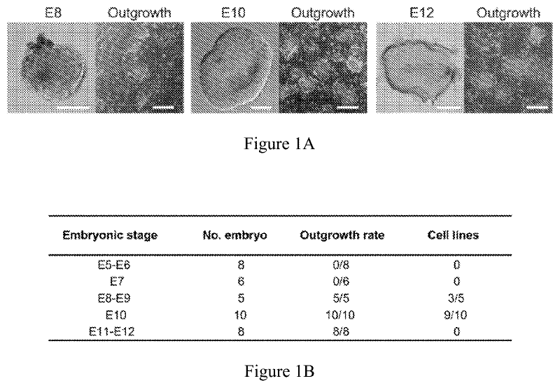

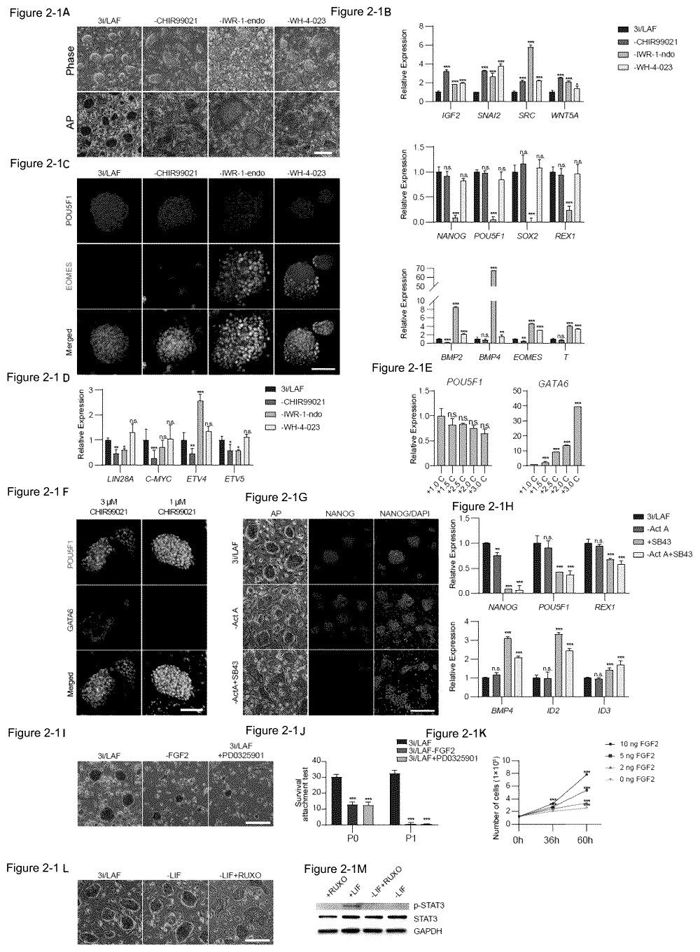

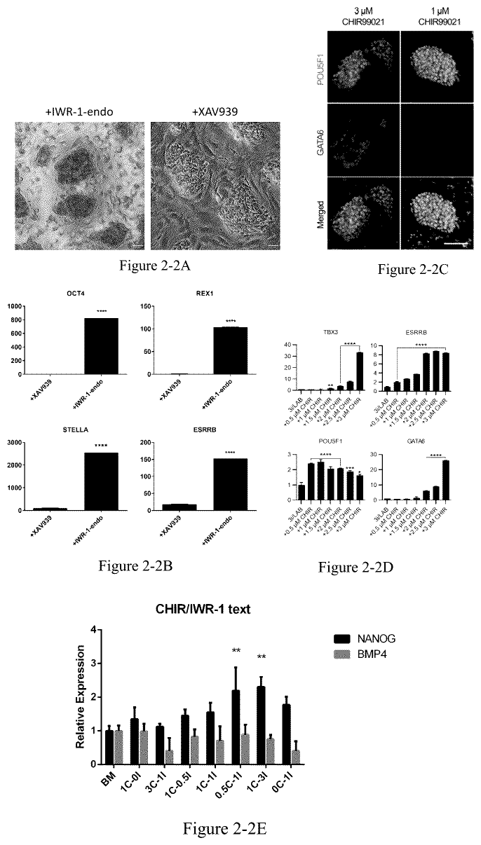

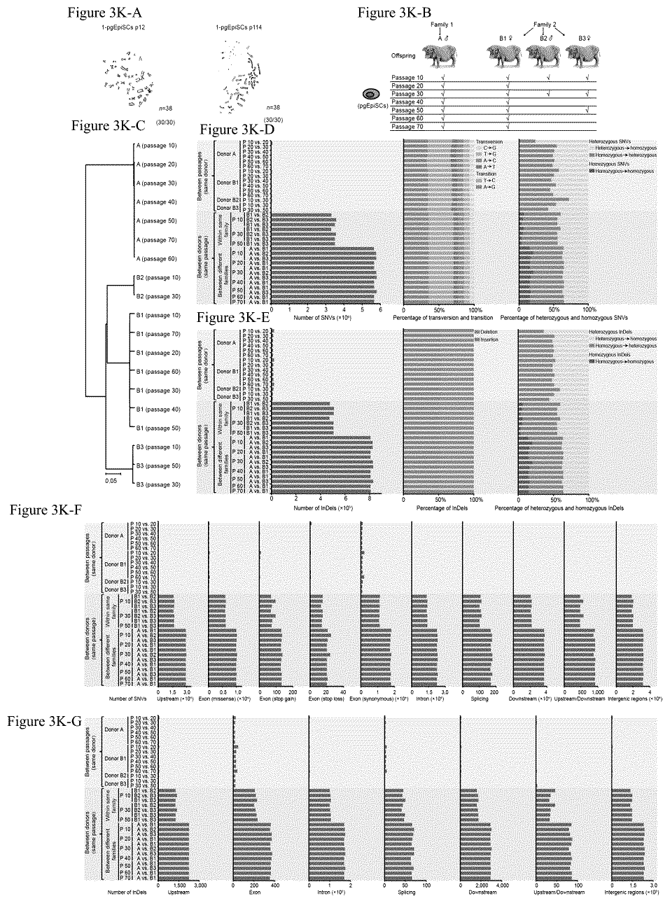

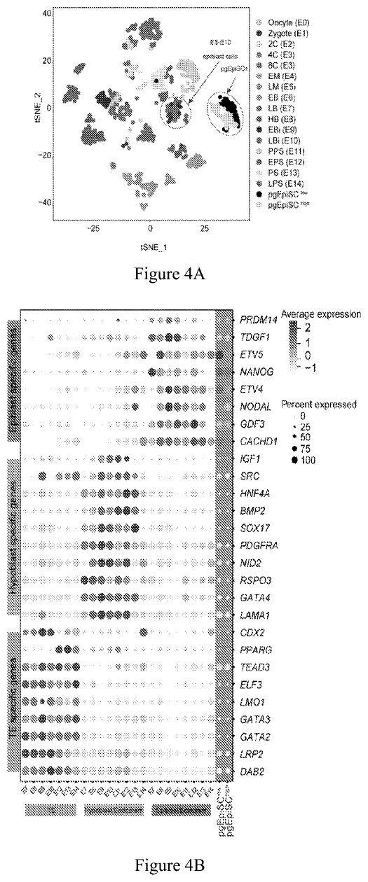

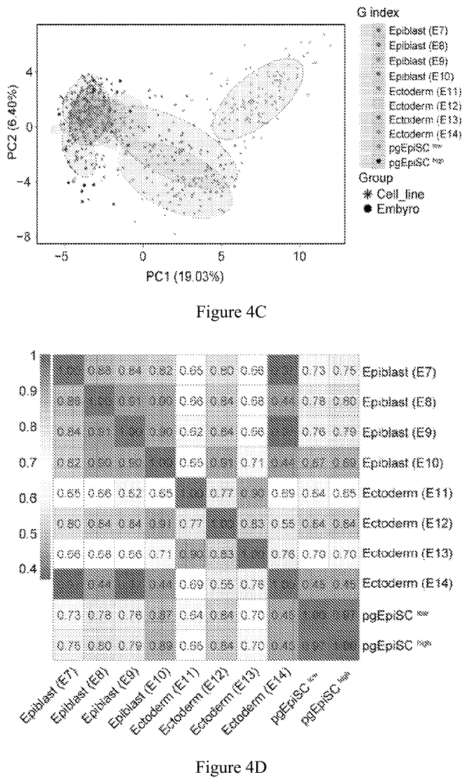

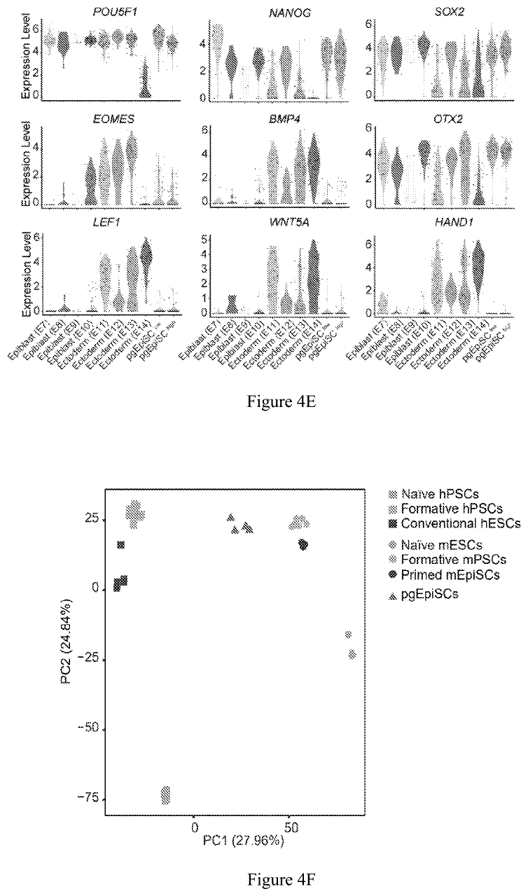

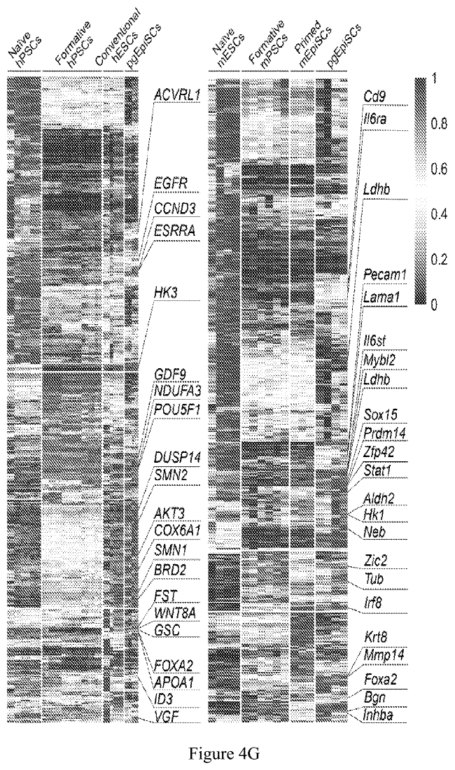

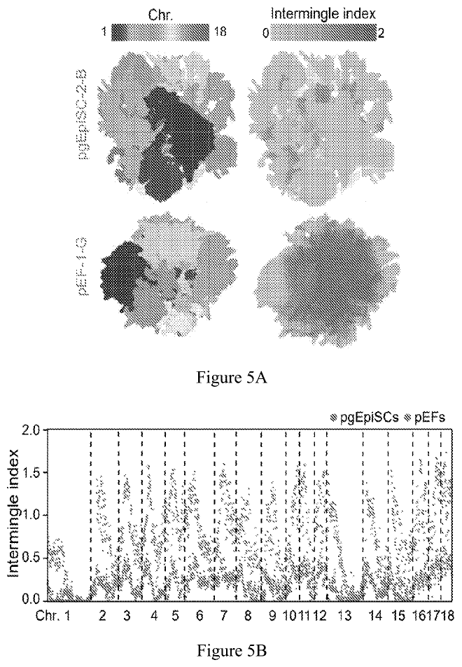

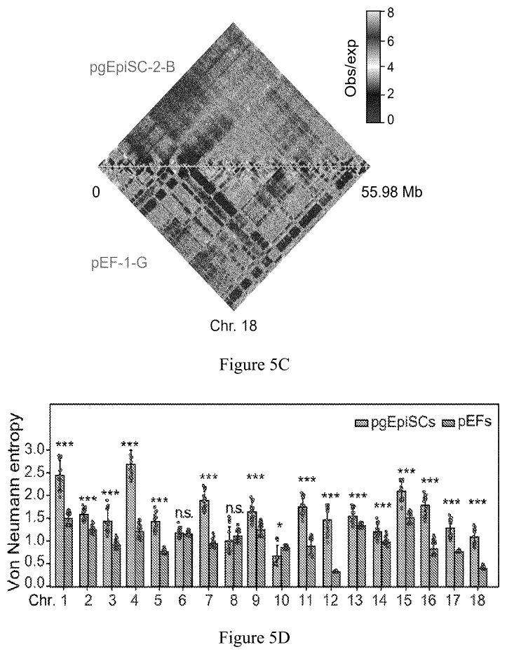

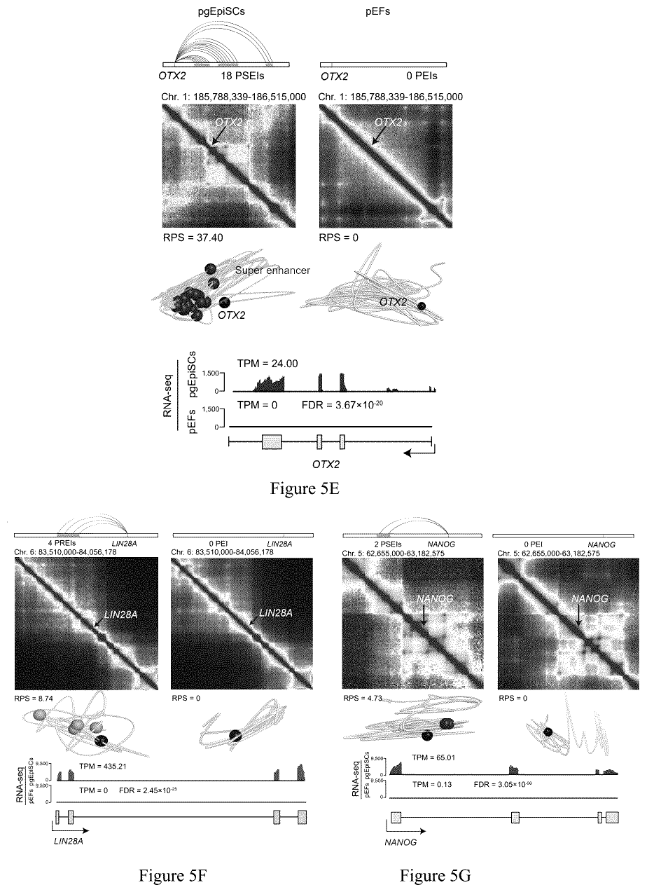

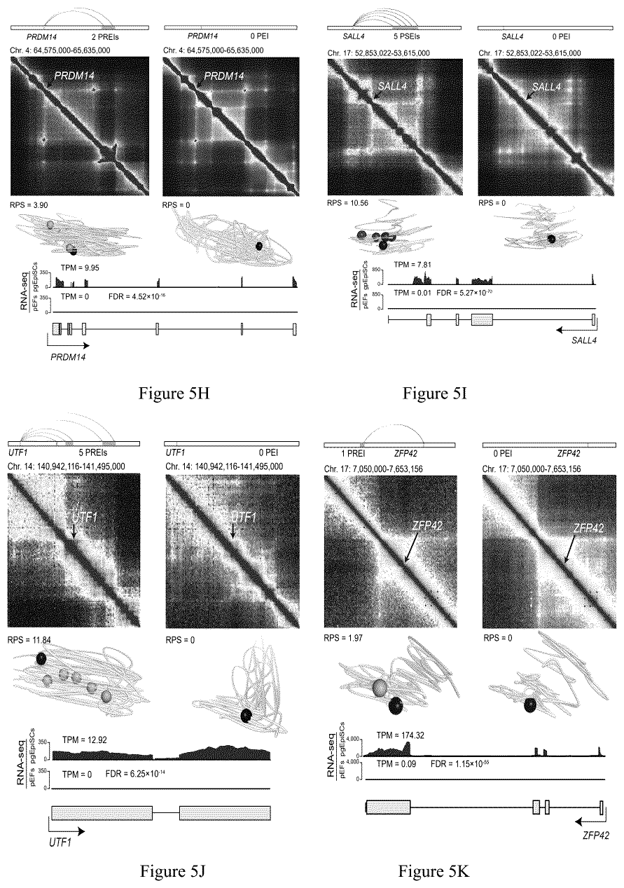

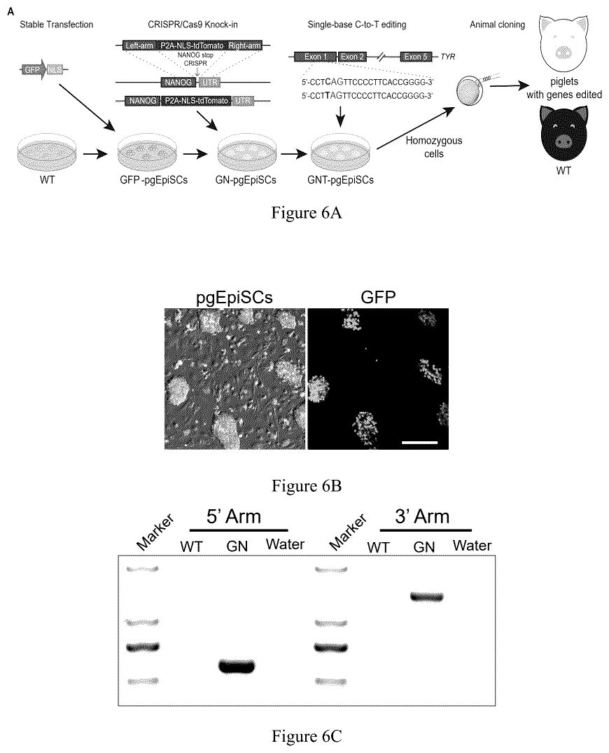

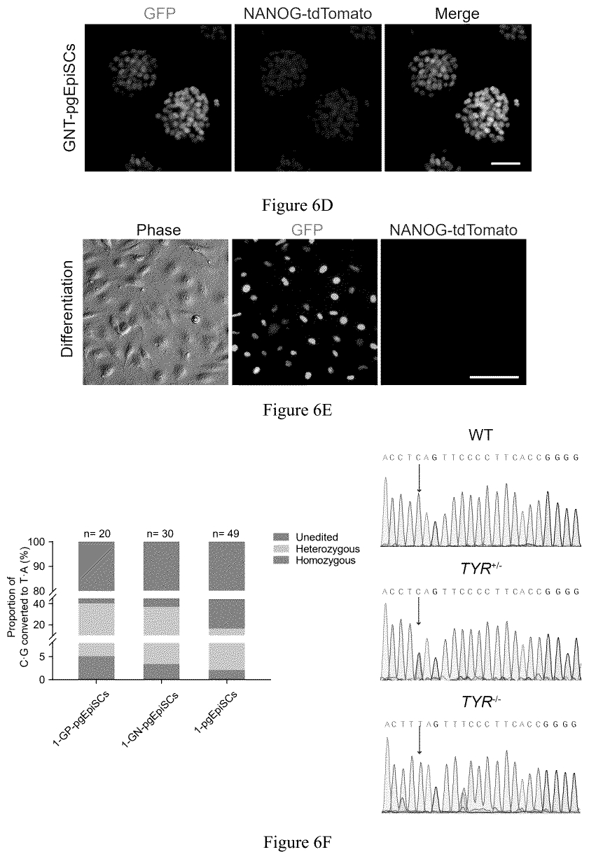

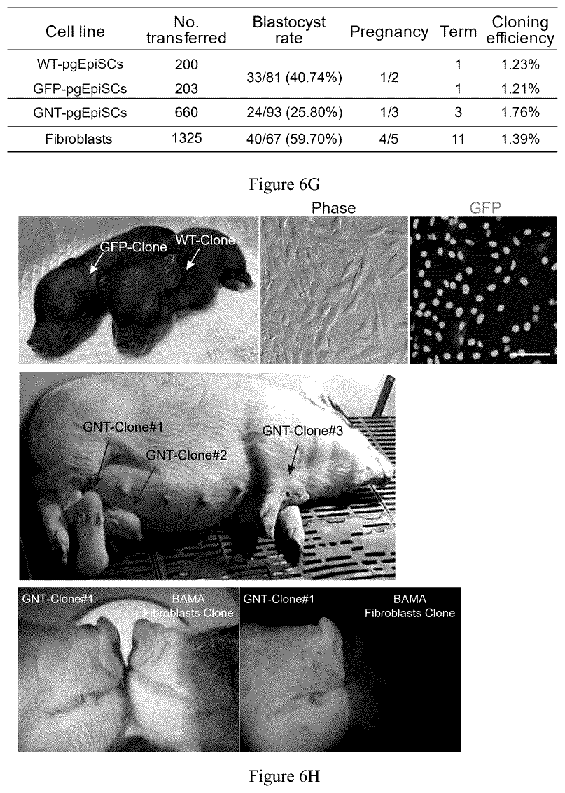

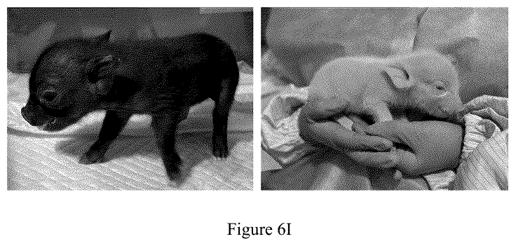

(FIG. 2-1A) Comparison of morphology and AP staining in pgEpiSCs cultured without CHIR99021, IWR-1-endo and WH-4-023. Scale bar, 200 μm.

(FIG. 2-1B) Quantification of mRNA expression of representative pluripotency marker genes involved in EMT (top), pluripotency (middle), and mesodermal differentiation (bottom) by qRT-PCR.

(FIG. 2-1C) Immunostaining for the pluripotency marker gene POU5F1 and mesoderm/endoderm progenitor marker EOMES. The nucleus is indicated by DAPI. Scale bar, 100 μm.

(FIG. 2-1D) Quantification of mRNA expression of proliferation-associated genes by qRT-PCR.

(FIG. 2-1E) Relative expression changes of POU5F1 and GATA6 in pgEpiSCs after treatment with different concentrations of CHIR99021.

FIG. 2-1 (F) Immunostaining for POU5F1 and GATA6 in pgEpiSCs treated with different concentrations of CHIR99021. The nucleus is indicated by DAPI. Scale bar, 100 μm.

(FIG. 2-1G) AP staining and immunostaining of pgEpiSCs cultured without Activin A (Act A) or with added SB431542 (SB43) compared with control 3i/LAF-cultured pgEpiSCs. Scale bar, 200 μm.

FIG. 2-1 (H) Quantification of mRNA expression of pluripotent genes and BMP4 signaling-related genes in culture medium without Act A and/or with added SB43.

(FIG. 2-1I) AP staining assay of pgEpiSCs cultured with or without FGF2 or with the ERK inhibitor PD0325901. Scale bar, 200 μm.

(FIG. 2-1J) Cell survival and attachment test of pgEpiSCs cultured with or without FGF2 or with the ERK inhibitor PD0325901.

(FIG. 2-1K) Cell proliferation curve of pgEpiSCs treated with different concentrations of FGF2.

(FIG. 2-1L) AP staining assay of pgEpiSCs cultured with or without LIF or with the JAK 1/2 inhibitor ruxolitinib (RUXO). Scale bar, 200 μm.

(FIG. 2-1M) Western blot analysis of LIF expression in pgEpiSCs during in vitro maintenance.

For (FIG. 2-1B), (FIG. 2-1D), (FIG. 2-1E), (FIG. 2-1H) and (FIG. 2-1J), error bars indicate±SD (n=3, independent experiments); n.s., P≥0.05; *, P<0.05, **, P<0.01, ***, P<0.001. For (FIG. 2-1A), (FIG. 2-1C), (FIG. 2-1F), (FIG. 2-1G), (FIG. 2-1I) (FIG. 2-1L) and (FIG. 2-1M), similar results were obtained in three independent experiments.

FIGS. 2-2A to 2-2E: Effect of different culture conditions on the preparation of pgEpiSC.

(FIG. 2-2A) Effect of IWR-1-endo replacement with XAV939 on the maintenance of cell pluripotency, with AP positivity lost at around 8 passages of passaging.

(FIG. 2-2B) Effect on the expression of core pluripotency factor OCT4 (POU5F1), pluripotency factors REX1, STELLA and ESRRB after IWR-1-endo replacement with XAV939.

(FIG. 2-2C) Effects of different concentrations of CHIR99021 on the expression of pluripotency gene POU5F1 and lineage specification-associated gene GATA6.

(FIG. 2-2D) Effects of different concentrations of CHIR99021 on the expression of pluripotency gene POU5F1, lineage specification-associated gene GATA6, mesoderm marker gene TBX3 and endoderm gene ESRRB.

(FIG. 2-2E) Effect of the concentration ratio of CHIR99021 to IWR-1-endo on the expression of pluripotency factor Nanog.

FIG. 2-3A: Effect of concentration adjustment of CHIR99021 in 3i/LAF medium on in vitro pluripotency of pgEpiSCs, the upper panel indicates before AP staining and the lower panel indicates after AP staining.

FIG. 2-3B: Effect of concentration adjustment of IWR-1-endo in 3i/LAF medium on in vitro pluripotency of pgEpiSCs, the upper panel indicates before AP staining and the lower panel indicates after AP staining.

FIG. 2-3C: Effect of concentration adjustment of WH-4-023 in 3i/LAF medium on in vitro pluripotency of pgEpiSCs, the upper panel indicates before AP staining and the lower panel indicates after AP staining.

FIG. 2-3D: Effect of concentration adjustment of recombinant human Activin A in 3i/LAF medium on in vitro pluripotency of pgEpiSCs, the upper panel indicates before AP staining and the lower panel indicates after AP staining.

FIG. 2-3E: Effect of concentration adjustment of recombinant human FGF-basic in 3i/LAF medium on in vitro pluripotency of pgEpiSCs, the upper panel indicates before AP staining and the lower panel indicates after AP staining.

FIG. 2-3F: Effect of concentration adjustment of recombinant human LIF in 3i/LAF medium on in vitro pluripotency of pgEpiSCs, the upper panel indicates before AP staining and the lower panel indicates after AP staining.

FIG. 2-3G: Effect of replacing CHIR99021 for WNT3a in 3i/LAF medium on the in vitro pluripotency of pgEpiSCs, the upper panel indicates before AP staining and the lower panel indicates after AP staining.

FIG. 2-3H: Effect of replacing WH-4-023 for A419259 in 3i/LAF medium on in vitro pluripotency of pgEpiSCs, the upper panel indicates before AP staining and the lower panel indicates after AP staining.

FIG. 2-3I: Effect of replacing recombinant human Activin A for Nodal in 3i/LAF medium on in vitro pluripotency of pgEpiSCs, the upper panel indicates before AP staining and the lower panel indicates after AP staining.

FIG. 3A: Cell proliferation curve of pgEpiSCs. The initial cell count was 2×10⁵.

FIG. 3B: Population doubling time of pgEpiSCs.

FIG. 3C: Single-cell cloning efficiency of pgEpiSCs.

FIG. 3D: Morphology of low- and high-passage pgEpiSC colonies. Scale bars, 200 μm.

FIG. 3E: Alkaline phosphatase (AP) staining assay for low and high passage numbers of pgEpiSC colonies. Scale bars, 200 μm.

FIG. 3F: Immunostaining of the pluripotency markers POU5F1, NANOG and SOX2 in pgEpiSCs. DAPI was used to stain nuclei. Scale bar, 50 μm.

FIG. 3G: Immunostaining of the pluripotency surface markers SSEA1, SSEA4, TRA-1-81 and TRA-1-60 in pgEpiSCs. DAPI was used for staining of cell nuclei. Scale bar, 50 μm.

FIG. 3H: In vitro EB differentiation assay. Immunostaining for the ectodermal neuro-specific marker protein Tubulin β-III, mesodermal muscle-specific marker protein α-SMA and endodermal specific marker protein GATA6. DAPI was used for nuclei staining. Scale bar, 50 μm.

FIG. 3I: Immunostaining of pgEpiSCs after directional induced differentiation. SOX1 is neural ectodermal marker, T is a mesodermal marker, GATA6 is an endodermal marker, and the nuclei are indicated by DAPI. Scale bar, 200 μm.

FIG. 3J: In vivo teratoma formation assay. Hematoxylin and eosin staining of teratomas derived from pgEpiSCs. Scale bar, 100 μm.

FIGS. 3K-A to 3K-G shows the characterizations of SNVs and Short InDels (≤30 bp) of pgEpiSC cell lines between different passages of a same cell line and between different cell lines.

(FIG. 3K-A) Karyotyping of low- and high-passage pgEpiSCs. 30 cells of per cell line were examined at metaphase.

(FIG. 3K-B) Schematic diagram of whole genome re-sequencing of 19 pgEpiSC samples (each sequenced to a depth of 24.42×) from four independent donor cell lines (A, B1, B2, B3). Notably, the three independent donor cell lines (i.e., B1, B2, and B3) are full siblings.

(FIG. 3K-C) A phylogenetic tree by Neighbor-joining (NJ) method of 19 pgEpiSC cell lines. Scale bar represents p-distance.

(FIG. 3K-D, FIG. 3K-E) Numbers and compositions of SNVs (d) and InDels (e). In contrast, there were a large number of mutations among pgEpiSCs from a same passage but different donors (between three full siblings: ˜3.46 M SNVs [Ts/Tv ratio: ˜2.41] and ˜498.10 K InDels; between different families: ˜5.71 M SNVs (Ts/Tv ratio: ˜2.41) and ˜816.72 K InDels). The number of mutations (˜37.98 K SNVs [Ts/Tv ratio: ˜2.13] and ˜15.58 K InDels) in pgEpiSCs from the same donor after multiple passages accounted for only a small fraction of the mutations (compared to the two full siblings, SNVs: ˜1.10%; InDels: ˜3.13%). In addition, compared to the proportion of homozygous mutations of pgEpiSCs from the same donor after multiple passages (SNVs: ˜0.08%; InDels: ˜0.22%), homozygous mutation among pgEpiSCs from different donors increased significantly (between three full siblings: SNVs: ˜5.22%, InDels: ˜4.96%; SNVs and InDels between different family donors were ˜16.67% and ˜16.02%, respectively).

(FIG. 3K-F, FIG. 3K-G) Summary and annotation of SNVs (f) and InDels (g) in different genomic elements. Site annotation was performed for each SNV and InDel gene using the ANNOVAR package.

FIG. 4A: t-SNE was plotted using scRNA-seq data from pig preimplantation embryonic cells (n=1458) and pgEpiSCs (n=196). Clusters are color-coded according to embryonic day and pgEpiSC passage number. Circled areas represent pre-gastrulation epiblast cells and pgEpiSCs.

FIG. 4B: Dot plot for classical marker genes of TE, hypoblasts and epiblasts during pig embryonic development. The color gradient represents the average expression level and the size of the dots corresponds to the percentage of cells expressing the featured genes in TE, hypoblast and epiblast cell populations.

FIG. 4C: PCA plot of pgEpiSCs and epiblast or ectoderm cells of E7˜E14. Each dot represents a single cell in preimplantation embryonic cells and asterisks represent single cells in pgEpiSCs. Colors denote embryonic day and pgEpiSC passage number.

FIG. 4D: Spearman's correlation coefficients are based on the average expression levels of uniquely expressed genes within each epiblast/ectoderm cell ranging from E7˜E14, which are associated with pluripotency regulation and epithelial cell differentiation.

FIG. 4E: Violin plots showing the expression levels (log 2 (TPM/10+1)) of classical pluripotency genes in E7-E14 and low- and high-passage pgEpiSCs based on scRNA-seq data. Each dot represents a single cell.

FIG. 4F: PCA Plot for naïve, formative and conventional hPSCs; naïve, formative and primed mPSCs; and pgEpiSCs, which is based on the set of uniquely expressed genes for each PSC. Colors represent pluripotency status. Triangles represent that from pig, squares represent from human, and circles represent from mouse.

FIG. 4G: Expression levels of uniquely expressed genes identified in naïve hPSCs, formative hPSCs, and conventional hESCs (left) and naïve mESCs, formative mPSCs, and primed mEpiSCs (right) compared to that of these genes in pgEpiSCs. The listed genes are highly expressed in pgEpiSCs.

FIG. 5A: Resolution of each Hi-C map is 100 kb intra-chromosomal and 1 mb inter-chromosomal, respectively. pgEpiSCs-1-B and pEF-1-G examples of cross sections of nuclei colored according to autosomes (left) or by the extent of the multichromosome intermingle index (reflecting chromosomal diversity measured by Shannon's index) (right) (Tan et al., 2018).

FIG. 5B: Probability of extensive multichromosome intermingling (averaged over 16 Hi-C maps for each cell type) across 18 autosomes (smoothed by a 1 mb windows) in pgEpiSCs (green) and pEFs (red) at 100 kb resolution (Tan et al., 2018).

FIG. 5C: Example contact maps of chromosome 18 in pgEpiSCs-1-B (upper half) and pEF-1-G (lower half) at 100 kb resolution.

FIG. 5D: The extent of disorder in chromatin structure (quantified by Von Neumann Entropy) (Lindsly et al., 2021) in each of the 16 Hi-C maps of pgEpiSCs (green) and pEFs (red) at 100 kb resolution.

FIG. 5E-5K: Schematic diagrams of PEIs for OTX2 (E), LIN28A (F), NANOG (G), PRDM14 (H), SALL4 (I), UTF1 (J), and ZFP42 (K). Top panel: Hi-C maps of regions around the center of the gene's transcriptional start site (+250 kb). Middle panel: 3D model of the promoter (blue sphere) and its enhancers (red and green spheres represent super-enhancers and regular enhancers, respectively). Bottom panel: RNA-seq profile. The Benjamini-Hochberg-adjusted FDRs were calculated.

FIG. 6A: Schematic diagram of the sequential editing strategy for multiple genes and generation of cloned piglets using pgEpiSCs as donors by cell nuclear transfer assay.

FIG. 6B: Morphology and fluorescence detection of the GFP-pgEpiSC clones, scale bar, 200 μm.

FIG. 6C: Identification of NANOG-tdTomato knock-in by PCR. “GN” stands for GFP positive NANOG-tdTomato knock-in pgEpiSC.

FIG. 6D: Expression of NANOG-tdTomato knock-in reporter gene in GFP-labeled pgEpiSCs. Scale bar, 100 μm.

FIG. 6E: Loss of tdTomato expression after differentiation of GN-pgEpiSCs. Scale bar, 200 μm.

FIG. 6F: Statistical data on the unedited, homozygous, and heterozygous ratios of the C to T mutation of the TYR gene in WT pgEpiSCs (1-pgEpiSCs) and genetically modified pgEpiSCs (1-GP-pgEpiSCs and 1-GN-pgEpiSCs) (left); representative DNA sequencing analysis of the C to T mutation site of the TYR gene in wild-type, heterozygous, and homozygous pgEpiSCs (right).

FIG. 6G: Summary of pgEpiSC nuclear transfer assays. Blastocyst rate was calculated using embryos retained prior to transfer. The fibroblasts were obtained from BAMA pigs.

FIG. 6H: Top panel: fibroblasts from the ears of WT pgEpiSCs cloned piglets and GFP-pgEpiSCs cloned piglets; Middle panel: 3 GNT-pgEpiSCs cloned piglets and their surrogate mothers; Bottom panel: representative GNT-pgEpiSCs cloned piglets showed GFP fluorescence, which was contrary to the WT piglets cloned from Bama pig fibroblasts.

FIG. 6I: Representative cloned piglets produced by using WT pgEpiSCs as donor cells (left) show black coat color, whereas representative cloned piglets produced by using GNT-pgEpiSCs as donor cells (right) show white coat albino phenotype.

### DETAILED DESCRIPTION

The present invention is now described with reference to the following examples intended to exemplify the invention (and not to limit the invention).

Unless specifically defined, the molecular biology experimental methods and immunoassays used in the present invention are referred to essentially as described in J. Sambrook et al, Molecular Cloning: A Laboratory Manual, 2nd Edition, Cold Spring Harbor Laboratory Press, 1989, and F. M. Ausubel et al, Short Protocols in Molecular Biology, 3rd Edition, John Wiley & Sons, Inc., 1995; restriction endonucleases were used in accordance with the conditions recommended by the product manufacturer. It is known to those skilled in the art that the examples describe the invention by way of example and are not intended to limit the scope of the invention.

Since the 1990s, researchers have been attempting to establish stable epithelial cell-derived stem cell lines from pigs, but have been unsuccessful. The inventors succeeded in establishing stable pig pre-gastrulation epiblast (Epiblast) stem cell lines (Pre-gastrulation epiblast stem cell lines, pgEpiSCs) through large-scale single-cell transcriptome analysis of pig embryos from day 0 to day 14. The inventors demonstrated that pgEpiSCs maintained pluripotency and normal karyotype after more than 200 passages, and revealed the functional impact of three-dimensional spatial associations on the transcriptional regulation of pluripotency marker genes in pgEpiSCs by high-deep in situ Hi-C analysis. In addition, the inventors also demonstrated that pgEpiSCs could well tolerate at least three consecutive rounds of gene editing and produced cloned live gene-edited piglets by somatic cell nuclear transfer. The inventors' discoveries offer hope for the long-awaited pig pluripotent stem cells and create a new path for biological research, animal husbandry and regenerative biomedicine.

### Experimental Methods

### 1. Preparation and Sequencing of a Single-Cell RNA Library

A scRNA-seq library was prepared by a modified Smart-seq2 protocol (Gao et al., 2018; Wang et al., 2018). Briefly, single embryonic cells were transferred into prepared lysis buffer containing an 8 bp bar code. Then, the first strand cDNA was synthesized and amplified by reverse transcription in a reverse transcription (RT) mixture containing 4U RNAase inhibitor, 100U SuperScript II reverse transcriptase (Invitrogen, 18064071), 1 mM dNTPs (TAKARA, 4019), 60 mM MgCl₂, and 3 μM RT primer with 10 μM TSO primer. The product after PCR amplification was purified using 0.8×AMPure XP beads (Beckman, A63882). Subsequently, biotin PCR amplification was performed. Finally, a single-cell RNA-seq library was constructed according to the PCR library Amplification/Illumina series KAPA Hyper Prep kit (KAPA, KK8054). High-quality libraries were sequenced on Illumina Hiseq Xten (Novogene) at 150 bp paired-end.

### 2. Cell Growth Curve and Population Doubling Time

pgEpiSCs were cultured in a 12-well plate. Cell samples were inoculated at a density of 2× 10⁴ cells per well in triplicate. The number of cells was counted every 12 hours. For each time point, cells were digested and counted using a Luna™ Automated Cell Counter, and the results of three counts were averaged and plotted. The cell doubling time was calculated as follows: doubling time (DT)=24×[lg2/(lgNₜ−lgN₀)], where 24 is the cell culture time (hour); Nₜ is the number of cells cultured for 48 hours; and No is the number of cells recorded at 24 hours.

### 3. Analysis of Single-Cell Cloning Efficiency

Cells were dissociated by Accutase (Gibco, A11105-01), counted using a haemocytometer, and inoculated in triplicate onto feeders of pre-inoculated 6-well plates at a density of 100, 200, and 1,000 cells per well under pgEpiSCs culture conditions. Clones were counted after 6 days using AP staining and the efficiency of clone formation was evaluated as a percentage of the number of clones per inoculated cell.

### 4. Karyotyping

Prior to karyotype analysis, 1% KaryoMAX Colcemid solution (Gibco, 15212012) was added into the pgEpiSCs medium and the cells were incubated for 1 hour. The pgEpiSCs were digested into single cells by TrypLE™ Express (Gibco, 12605010) and collected by centrifugation. The pgEpiSCs were resuspended with 0.075 M KCl (sigma, P5405) hypotonic solution and incubated at 37° C. for 15 min. The pgEpiSCs were then fixed with methanol and acetic acid at a ratio of 3:1, and the process was repeated three times. The suspension of pgEpiSCs was dropped onto pre-cooled slides, dried thoroughly at room temperature, and then stained with 10% Giemsa staining solution (Sangon, E607314-0001) for 30 min. For each cell line, more than 30 cells were examined at metaphase.

### 5. Whole Genome Sequencing

Total DNA from pgEpiSCs was extracted using the TIANamp Genomic DNA Kit (TIANGEN, DP304). After DNA extraction, 1 μg of genomic DNA was randomly fragmented using Covaris and selected for whole genome sequencing using the Agencourt AMPure XP-Medium Kit (BERCKMAN COULTER, A63880, A63880) to select 200-400 bp fragments. The selected fragments were end-repaired and 3′ adenylated, and then the adapters were ligated to the ends.

The product was amplified by PCR, and then the purified PCR product was thermally denatured into single strand and looped by a splint oligo sequence. The single-stranded circular DNA was formatted into a final library and verified by quality control. The final verified library was sequenced by BGISEQ-500.

### 6. Alkaline Phosphatase (AP) Staining

The alkaline phosphatase staining of pgEpiSC was based on the Alkaline Phosphatase Assay Kit (Millipore, SCR004). Specific experimental steps followed the instructions of the kit.

7. Immunofluorescence analysis

Cells were washed with DPBS (Gibco, C14190500BT) and fixed with 4% paraformaldehyde for 30 min at room temperature, then washed with DPBS again, permeabilized in 0.1% Triton X-100 for 20 min and blocked with 3% BSA for 1 hour. Cells were incubated with primary antibody diluted with 3% BSA at 4° C. overnight. Cells were then washed three times for 3 min each with wash buffer (DPBS containing 0.1% Triton X-100 and 0.1% Tween 20). The secondary antibody was diluted, and incubated with wash buffer for 1 hour at room temperature, then washed three times for 5 min each with wash buffer, and then the nuclei were stained with DAPI (Roche Life Science, 10236276001) for 3 min.

### 8. Embryoid Body Differentiation

pgEpiSCs were dissociated by Accutase (Gibco, A11105-01), separated from feeder cells using a differential attachment method, and cultured on 35-mm low-attachment plates placed on a horizontal shaker at 50 rpm with the addition of 10% FBS (Gibco, 11960-044), 1% penicillin-streptomycin (Thermo Fisher Scientific, 15960-01) and 1% glutamine (Thermo Fisher Scientific, 35050-061) in DMEM (Gibco, 11960-044) for 5-7 days. Regular spherical EBs were selected and plated on gelatin-coated plates in the same medium within 1 week, then fixed and detected using the same methods as for immunofluorescence.

### 9. Directed Induced Differentiation

For neural induction, the 3i/LAF medium was replaced with neural induction medium I (2.5 μM IWR-1-endo, 5 μM SB431542 and 10 ng/ml FGF2 in BM) two days after the passage of pgEpiSCs. After 2 days of incubation, the medium was replaced with Neural Induction Medium II (4 μM RA, 10 ng/mL FGF2 and 20 ng/ml Noggin in BM), and immunostaining was performed after 2 days.

For endoderm induction, the 3i/LAF medium was replaced with 10 ng/mL BMP4, 5 μM SB431542, and 10 ng/ml FGF2 two days after pgEpiSCs passage. The immunostaining was performed after passage.

For mesoderm induction, the 3i/LAF medium was replaced with mesoderm induction medium I (10 ng/mL BMP4, 50 ng/ml Activin A and 20 ng/ml FGF2 in BM) two days after pgEpiSCs passage for a two days' culture. Subsequently, mesoderm induction medium II (3 μM IWR-1-endo, 5 μM CHIR99021, and 20 ng/ml FGF2 in BM) was added, and immunostaining was performed after 2 days.

### 10. Teratoma Formation

For the teratoma formation assay, approximately 1×10⁷ dissociated pgEpiSCs cells were collected by centrifugation at 1,000 rpm for 5 min and subcutaneously injected into the posterior neck of BALB/c nude mice. Teratomas were visible after 4-5 weeks of feeding.

### 11. H&E Analysis

Teratomas were collected subcutaneously from nude mice, washed twice in PBS and fixed with 4% PFA at 4° C. for 2 days. Teratoma tissues were dehydrated with alcohol an gradient (70%, 80%, 90%, 95% and 100% for 1 h each), transferred into xylene and embedded in paraffin. The samples were sliced to a thickness of 5 μm, deparaffinized in xylene and rehydrated with decreasing concentrations of ethanol. Samples were then stained with hematoxylin (Sigma-Aldrich, MHS16) and eosin (Sigma-Aldrich, HT110116) and observed under a microscope (Leica, DM5500B).

### 12. RT-qPCR

Total RNA from pgEpiSCs was extracted using the RNAprep Pure Cell/Bacteria Kit (TIANGEN, DP430), and then reverse transcribed to cDNA using 5×All-in-One RT Master Mix (Abm, G490). PCR was performed on a LightCycler480 II Real Time System (Roche) using 2×RealStar Green Power Mixture (GenStar, A311-05). The data were analyzed using the comparative CT (2^{-ΔΔCT}) method. ΔCT was calculated using EF1A as an internal control. All experiments were performed in three biological replicates. Primers used for quantitative real-time PCR are listed in the Resource table.

### 13. Western Blot Analysis

Total proteins were extracted from cells by cell lysis buffer (Beyotime Biotechnology, P0013), and nuclear and cytoplasmic proteins were extracted using the nucleus and cytoplasmic protein extraction kit (Beyotime Biotechnology, P0027) supplemented with protease and phosphatase inhibitor cocktail (Beyotime Biotechnology, P1050) to extract nuclear and cytoplasmic proteins. The concentration of extracted proteins was measured using the Bradford Protein Assay Kit (Bio-red, 5000201). An equal amount of proteins (15 μg) was separated by SDS-PAGE gel electrophoresis, which were transferred from the gel to Immobilon-P transfer membrane (Merck Millipore; pore size: 0.45 μm; IPVH00010). The blots were blocked in 5% nonfat powdered milk (Sangon Biotech, A600669-0250) in TBST (20 mM Tris, pH 7.5; 150 mM NaCl; 0.1% Tween 20) at room temperature for 1 hour and then incubated with primary antibodies diluted in 5% nonfat powdered milk in TBST overnight at 4° C. The next day, the blots were rinsed three times with TBST for 5 minutes each time, then incubated together with HRP-coupled secondary antibody diluted in 5% nonfat powdered milk in TBST for 1 hour at room temperature. The blots were developed with SuperSignal West Dura Extended Duration Substrate (Thermo Fisher Scientific, 34075), and the intensity of the bands of the target proteins was analyzed using CLINX chemiluminescence software. Specific experimental methods and reagent formulations were obtained from the Western Blotting General Protocol (Bio-red, bulletin 6376). Specific experimental methods and reagent formulations were derived from the General Protocol for Western Blotting (Bio-red, bulletin 6376).

### 14. In Situ Hi-C

Four Hi-C libraries (as technical replicates) for each of the four pgEpiSCs (biological replicates) and eight Hi-C libraries (as technical replicates) for each of the two pEFs (biological replicates) were constructed according to the previously published In situ Hi-C method (see, Rao, S. S. et al., A 3D map of the human genome at kilobase resolution reveals principles of chromatin looping. Cell 159, 1665-1680.) with some minor modifications.

Briefly, cells (5×10⁶) were cross-linked with formaldehyde at a final concentration of 4% for 30 mins at room temperature and then quenched with glycine at a final concentration of 0.25 M/L. The mixtures were centrifuged next at 1,500×g for 10 min at room temperature, and the supernatants were combined with the lysis buffer and incubated on ice for 15 min. The mixture was then centrifuged at 5,000×g for 10 min at room temperature. The precipitate was washed with NEBuffer 2. The mixture was combined with SDS at a final concentration of 0.1% and incubated at 65° C. for 10 minutes, then TritonX-100 was added at a final concentration of 1% and incubated at 37° C. for 15 minutes. Nuclei were permeabilized, and DNA was digested with 200 units of DpnII for 1 hour at 37° C. The restriction fragment overhangs were filled and labeled with biotinylated nucleotides and then ligated in small volumes. After crosslink reversal, DNA was purified and sonicated to fragments of approximately 300-500 bp using a Covaris S220 sonicator, at which point ligated fragments were pulled down with Dynabeads™ M-280 Streptavidin (Invitrogen, 11206D), end-repaired and A-tailed. Adaptors were next ligated, and DNA fragments were PCR amplified using a KAPA Hyper Prep Kit (Roche, KK8504) for 8-10 cycles. These fragments were then double-selected using AMPure XP Beads (Beckman, A63882) to isolate fragments between 300 and 800 bp, which were prepared for sequencing on the DNBSEQ platform (BGI) to provide 100 bp paired-end reads (Data S1, IA).

### 15. RNA Sequencing

pgEpiSCs from four donors (as biological replicates) and pEFs from the same dorsal skin region from two donors (as biological replicates) were collected and purified, each replicate having 1×10⁶ cells. Total RNA was extracted from each of the six samples (four pgEpiSCs and two pEFs) using the RNeasy Mini Kit (Qiagen, 74106). A IRNA depletion protocol (Globulin-Zero Gold IRNA Removal Kit, Illumina, GZG1224) coupled with the NEBNext& Ultra™ Directional RNA Library Prep Kit for Illumina® (NEB, E7420S) to construct the strand-specific RNA-seq library for each sample. All libraries were quantified using the Qubit dsDNA High Sensitivity Assay Kit (Invitrogen, Life Technologies, Q32851) and sequenced on a HiSeq 4000 platform (Illumina) (Data S1, IB).

### 16. ChIP-Seq

ChIP-seq of H3K27ac (a canonical histone marker of enhancers) of pgEpiSCs and pEFs was performed in two biological replicates, 1×10⁷ cells per sample. Cells were cross-linked with formaldehyde at a final concentration of 1% for 10 min at room temperature and then quenched with glycine. Cells were lysed with lysis buffer supplemented with protease inhibitor mixture and 1 mM PMSF (1×final each), and then approximately 200-500 bp fragments were sonicated using a Bioruptor. 20 μL of chromatin was stored at −20° C. for input DNA, and 100 μL of chromatin was immunoprecipitated with 5 μg of H3K27ac antibody (Abcam, ab4729) at 4° C. Then, 30 μL of protein beads were added and the samples were further incubated for 3 hours. The beads were then washed once with 20 mM Tris/HCl (pH 8.1), 50 mM NaCl, 2 mM EDTA, 1% Triton X-100, 0.1% SDS. The beads were treated twice with 10 mM Tris/HCl (pH 8.1), 250 mM LiCl, 1 mM EDTA, 1% NP-40 and 1% deoxycholic acid; and washed twice with 1×TE buffer (10 mM Tris-Cl at pH 7.5. 1 mM EDTA). Bound material was then eluted from the magnetic beads in 300 μL of elution buffer (100 mM NaHCO₃, 1% SDS) and treated first with RNAase A at a final concentration of 8 μg/mL for 6 hours at 65° C. and then with proteinase K (final concentration 345 μg/mL) overnight at 45° C. Sequencing libraries were constructed using immunoprecipitated DNA according to the protocol provided with the NEXTflex™ ChIP-Seq Kit (Bioo Scientific, NOVA-5143-02). All libraries were sequenced on the HiSeq XTen (Illumina) platform (Data S1, IB).

### 17. Vector Construction

To test whether pgEpiSCs could withstand consecutive gene editing, three gene editing experiments were performed in pgEpiSCs using different gene editing techniques, which are as follows: 1) stable transfection of GFP-NLS cassettes using the PiggyBac (PB) transposase tool; 2) NANOG-tdTomato reporter knock-in via CRISPR/Cas9 systems; and 3) TYR gene point mutation with Cytidine Base Editors (CBEs).

First, to generate GFP-positive cells, the PB-CMV-EF1A-GFP-NLS plasmid (from Prof. WU Sen of China Agricultural University) was constructed, replaced the chicken β-actin promoter with the Homo sapiens elongation Factor 1 alpha (EF1A) promoter and inserted a GFP-NLS into the end of the EFIA promoter. Second, to generate a NANOG-tdTomato KI cell line, a NANOG DNA donor vector backbone with four fragments were constructed, left homology arm-3×Flag, 3×Flag-P2A-tdTomato-Loxp-Puro-Loxp and right homology arm, using NEBuilder® HiFi DNA Assembly Master Mix (NEB, E2621X) as previously described. NANOG The sgRNA was targeted before the stop codon site to knock in the donor fragment as a reporter gene. The annealed sgRNA sequence was cloned into the Bsa I-digested pGL3-U6-sgRNA-PGK-puromycin vector (Addgene, 51133). Finally, the TYR gene was knocked out using the AncBE4max plasmid. AncBE4max vector (RRID: Addgene_112094) and pGL3-U6-sgRNA-EGFP vector (RRID: Addgene_107721) were obtained from the laboratory of HUANG Xingxu at the University of Science and Technology of Shanghai, China. sgRNA was synthesized by BGI with the ACCG sequence at the 5′ end of the forward primer and the AAAC sequence at the 5′ end of the reverse primer. Then, sgRNA was annealed and cloned into the pGL3-U6-sgRNA-EGFP vector. The details of sgRNA sequences are provided in Resource Table.

### 18. Cell Electrotransfection

Prior to electroporation, pgEpiSCs were dissociated using the Accutase Cell Dissociation Reagent (Gibco, A11105-01). For each electroporation, 5×10⁵ cells were transfected at 220 V, 5 ms, 2 pulses using BTX ECM 2001 (Harvard Bioscience, Holliston, MA, USA). For GFP-NLS cassette stable transfection, electroporation was performed using 1 μg of PBase helper plasmids and 3 μg of PB-CMV-EF1A-GFP-NLS donor plasmids (mass ratio 1:3). For NANOG-tdTomato reporter knock-in, electroporation was performed using 1 μg of pST1374-NLS-flag-linker-Cas9 plasmids (Addgene, 44758), 1 μg of NANOG sgRNA plasmids, and 1 μg of NANOG HMEJ donor plasmids (1 μg of each vector); and for the TYR gene point mutation, electroporation was performed with 2 μg of ancBE4max vector and 2 μg of pGL3-U6-TYR sgRNA-GFP vector (mass ratio 1:1). Electrotransfection buffer was provided by WU Sen's laboratory at the State Key Laboratory of Agricultural Biotechnology, China Agricultural University. Primers were designed online using NCBI primer BLAST and synthesized by BGI. GFP-positive cells were sorted using FACS (MoFlo XDP, Backman) and detected bandpass using a 488 nm (710/50 filter) channel. To generate NANOG-tdTomato-positive cells, transfected cells were selected with puromycin (0.3 μg/mL) and blasticidin (4 μg/mL), and GFP-positive clones were selected and amplified. To identify the base-edited cells, DNA was extracted using cell lysis buffer (Invitrogen, AM8723) as PCR template. PCR products were sequenced to confirm point mutations.

19. Generation of pgEpiSCs Cloned Embryos from Pig

Ovaries were collected from slaughterhouses around Beijing. Oocytes with three or four layers of cumulus cells were selected and cultured in IVM solution at 38.5° C., 100% humidity, and 5% CO₂ for 44 hours. IVM mother liquor M199 (Sigma, M2154) containing 0.1% L-cysteine (Sigma, C7352-25G), 5% FBS (Gibco, 10099141), 0.1% EGF (Sigma, E9644), and 1% penicillin-streptomycin (Gibco, 15140122) and 10% pig follicular fluid (follicular fluid was collected during oocyte collection, centrifuged and filtered, and then stored at −80° C.). After preparation, the IVM mother liquor was filtered through a 0.22 μm filter and stored at 4° C. for later use. Before use, 1% GlutaMAX (Gibco, 35050061), 10 IU/mL PMSG, and 10 IU/mL hCG were added. Pig EpiSCs were differentiated in basal medium containing 10 ng/ml BMP4, 5 μM SB431542, and 10 ng/ml FGF2 for more than 1 week, and then used as donor cells for nuclear transfer. Mature oocytes at metaphase II were enucleated by micromanipulation in HM medium containing 7.5 μg/mL cytochalasin B. Morphologically qualified donor cells were injected into the perivitelline space and fused with the recipient cytoplasm in fusion medium (0.3 M/L mannitol, 1.0 mM/L CaCl₂), 0.1 mM/L MgCl₂, and 0.5 mM/L HEPES) using a BLS Electro-cell Manipulator.

The oocytes were then incubated in PZM-3 for 15 min, and the fusion percentage was evaluated under a stereomicroscope. Fifty to sixty fused embryos were placed into four-well Petri dishes containing 500 μL of PZM-3 per well and incubated in PZM-3 in 5% CO₂, 5% O₂ and 90% N₂ with maximum humidity at 38.5° C. After 24 hours, 150-250 ESCNT zygotes were transferred surgically into the uterus of a surrogate mother. The pregnancy status of the surrogates was diagnosed by ultrasonography at 25-30 days. All cloned piglets were delivered s by natural birth on days 114-120 of gestation.

Resource table

| | | |
|---|---|---|
| REAGENT or RESOURCE | SOURCE | IDENTIFIER |

Antibodies

| | | |
|---|---|---|
| Rabbit polyclonal anti-neuron specific beta | Abcam | Cat# ab18207, |
| III Tubulin | | RRID: AB_444319 |
| Rabbit polyclonal anti-alpha smooth muscle | Abcam | Cat# ab5694, |
| Actin | | RRID: AB_2223021 |
| Rabbit monoclonal anti-GATA-6 (D61E4) | Cell Signaling Technology | Cat# 5851, |
| | | RRID: AB_10705521 |
| Mouse monoclonal anti-SSEA1 (MC480) | Abcam | Cat# ab16285, |
| | | RRID: AB_870663 |
| Mouse monoclonal anti-SSEA4 (MC813) | Abcam | Cat# ab16287, |
| | | RRID: AB_778073 |
| Mouse monoclonal anti-human-TRA-1-60 | Cell Signaling Technology | Cat# 4746, |
| | | RRID: AB_2119059 |
| Mouse monoclonal anti-human-TRA-1-81 | Cell Signaling Technology | Cat# 4745, |
| | | RRID: AB_2119060 |
| Rabbit polyclonal anti-SOX1 | Abcam | Cat# ab87775, |
| | | RRID: AB_2616563 |
| Rabbit polyclonal anti-Brachyury/Bry | Abcam | Cat# ab20680, |
| | | RRID: AB_727024 |
| Rabbit polyclonal anti-H3K27ac | Abcam | Cat# ab4729; |
| | | RRID: AB_2118291 |
| Rabbit polyclonal anti-TBR2/Eomes | Abcam | Cat# ab23345, |
| | | RRID: AB_778267 |
| Mouse monoclonal anti-Oct-3/4 (C-10) | Santa Cruz Biotechnology | Cat# sc-5279, |
| | | RRID: AB_628051 |
| Mouse monoclonal anti-Sox-2 (E-4) | Santa Cruz Biotechnology | Cat# sc-365823, |
| | | RRID: AB_10842165 |
| Rabbit polyclonal anti-human Nanog | Pepro Tech | Cat# 500-P236, |
| | | RRID: AB_1268274 |
| Rabbit monoclonal anti-Phospho-Stat3 | Cell Signaling Technology | Cat# 52075, |
| (Tyr705) (D3A7) | | RRID: AB_2799407 |
| Rabbit monoclonal anti-Stat3 (D3Z2G) | Cell Signaling Technology | Cat# 12640S, |
| | | RRID: AB_2629499 |
| Rabbit monoclonal anti-GAPDH (D16H11) | Cell Signaling Technology | Cat# 5174, |
| | | RRID: AB_10622025 |
| Goat anti-Mouse IgG (H+L) Cross-Adsorbed | Thermo Fisher Scientific | Cat# A-11001, |
| Secondary Antibody, Alexa Fluor 488 | | RRID: AB_2534069 |
| Goat anti-Mouse IgG (H+L) Cross-Adsorbed | Thermo Fisher Scientific | Cat# A-11005, |
| Secondary Antibody, Alexa Fluor 594 | | RRID: AB_141372 |
| Donkey anti-Rabbit IgG (H+L) Highly Cross- | Thermo Fisher Scientific | Cat# A-21207, |
| Adsorbed Secondary Antibody, Alexa Fluor 594 | | RRID: AB_141637 |
| Donkey anti-Rabbit IgG (H+L) Highly Cross- | Thermo Fisher Scientific | Cat# A-21206, |
| Adsorbed Secondary Antibody, Alexa Fluor 488 | | RRID: AB_2535792 |

Chemicals, Peptides, and Recombinant Proteins

| | | |
|---|---|---|
| CHIR99021 | Selleckchem | Cat# S1263 |
| IWR-1-endo | Selleckchem | Cat# S7086 |
| Y-27632 | Selleckchem | Cat# S1049 |
| WH-4-023 | Selleckchem | Cat# S7565 |
| SB431542 | Selleckchem | Cat# S1067 |
| Ruxolitinib (INCB018424) | Selleckchem | Cat# S1378 |
| PD0325901 | Selleckchem | Cat# S1036 |
| Recombinant Murine BMP-4 | Pepro Tech | Cat# 315-27 |
| Human/Murine/Rat Activin A (E.Coli) | Pepro Tech | Cat# 120-14E |
| Recombinant Human LIF | Pepro Tech | Cat# 300-05 |
| Recombinant Human FGF-basic (154 a.a.) | Pepro Tech | Cat# 100-18B |
| All-Trans Retinoic Acid | Pepro Tech | Cat# 3027949 |
| Recombinant Human Noggin | Pepro Tech | Cat# 120-10C |
| Bovine Serum Albumin | Sigma-Aldrich | Cat# A1470 |
| Ascorbic Acid | Sigma-Aldrich | Cat# A4544 |
| KnockOut Serum Replacement | Thermo Fisher Scientific | Cat# A3181502 |
| NeurobasalTM Medium | Thermo Fisher Scientific | Cat# 21103-049 |
| DMEM/F-12, GlutaMAXTM supplement | Thermo Fisher Scientific | Cat# 10565-018 |
| N-2 Supplement (100 ×) | Thermo Fisher Scientific | Cat# 17502-048 |
| B-27TM Supplement (50 ×), minus vitamin A | Thermo Fisher Scientific | Cat# 12587-010 |
| GlutaMAXIM Supplement | Thermo Fisher Scientific | Cat# 35050-061 |
| MEM Non-Essential Amino Acids Solution | Thermo Fisher Scientific | Cat# 11140-050 |
| (100 ×) | | |
| 2-Mercaptoethanol | Thermo Fisher Scientific | Cat# 21985-023 |
| Penicillin-Streptomycin (10,000 U/mL) | Thermo Fisher Scientific | Cat# 15140-122 |
| Gelatin (0.1% in water) | Stem Cell Technologies | Cat# 07903 |
| Trypsin-EDTA (0.05%), phenol red | Gibco | Cat# 25300120 |
| DMEM, high glucose, no glutamine | Gibco | Cat# 11960-044 |
| Fetal bovine serum (FBS) | Gibco | Cat# 16000-044 |
| Accutase cell dissociation reagent | Gibco | Cat# A11105-01 |
| TrypLE TM Express | Gibco | Cat# 12605010 |
| Dulbecco's phosphate-buffered saline (DPBS) | Gibco | Cat# C14190500CP |

Critical Commercial Assays

| | | |
|---|---|---|
| KAPA Hyper Prep Kits | KAPA Biosystems | Cat# KK8054 |
| RNeasy Mini Kit | QIAGEN | Cat# 74106 |
| Globin-Zero Gold rRNA Removal Kit | Illumina | Cat# GZG1224 |
| NEBNext ® Ultra ™ Directional RNA Library | New England Biolabs | Cat# E7760 |
| Prep Kit for Illumina ® | | |
| NEXTflex ™ ChIP-Seq Kit | Bioo Scientific | Cat# NOVA-5143-02 |
| RNAprep pure Cell/Bacteria Kit | TIANGEN | Cat# DP430 |
| 5 × All-In-One RT Master Mix | ABM | Cat# G490 |
| TIANamp Genomic DNA Kit | TIANGEN | Cat# DP304 |
| Rapid Giemsa staining kit | BBI Life Sciences | Cat# E202FA0001 |
| Gel & PCR Clean Up Kit | OMEGA | Cat# D2000-02 |
| Lipofectamine ™ 3000 Transfection Kit | Invitrogen | Cat# 2173193 |
| Lipofectamine ™ Stem Transfection Reagent | Invitrogen | Cat# STEM00008 |
| Endo-free Plasmid Mini Kit | OMEGA | Cat# D6950-02B |
| Bradford Protein Assay Kit | BIO-RED | Cat# 5000201 |
| 2 × RealStar Green Power Mixture | GenStar | Cat# A311-05 |
| Cells-to-cDNATM II Kit | Invitrogen | Cat# AM8723 |

Deposited Data

| | | |
|---|---|---|
| Human ESCs Hi-C data | (Dixon et al., 2015; | GEO: GSE52457, |
| | Lya et al., 2018) | GSE: 105028 |
| Human dermal fibroblasts Hi-C data | (Nir et al., 2018) | GEO: GSE123552 |
| Mouse ESCs Hi-C data | (Bonev et al., 2017; | GEO: GSE96107, |
| | McLaughlin et al, 2019) | GSE: 124342 |
| Mouse embryonic fibroblasts Hi-C data | (Colognori et al., 2019; | GEO: GSE113339 |
| | Di Giammartino et al., | |
| | 2019) | |
| Human ESCs RNA-seq data | (Ji et al, 2016) | GEO: GSE69692 |
| Human dermal fibroblasts RNA-seq data | (Consortium, 2012) | GEO: GSE78670 |
| Mouse ESCs RNA-seq data | (Shukla et al., 2020) | GEO: GSE121171 |
| Mouse embryonic fibroblasts RNA-seq data | (Di Giammartino et al., | GEO: GSE113431 |
| | 2019) | |
| Human formative PSCs, mouse naive, formative | (Kinoshita et al., 2021) | GEO: GSE131556 |
| and primed PSCs RNA-seq data | | |
| Human naïve and conventional PSCs RNA-seq | (Guo et al., 2017) | ArrayExpress: |
| data | | E-MTAB-5674 |
| Human naïve and conventional PSCs RNA-seq | (J) et al., 2016) | GEO: GSE69692 |
| data | | |

Experimental Models: Organisms

| | | |
|---|---|---|
| CD-1 ® (ICR) IGS Mice | Vital River | 201 |
| BALB/c Nude Mice | Vital River | 401 |
| BAMA pigs | Beijing Farm Animal | N/A |
| | Research Center | |
| Nongda pigs | CAU Experimental Miniature | N/A |
| | Pig Farm | |
| DLY pigs | YAU/NEAU Experimental Pig | N/A |
| | Farm | |

Oligonucleotides

| | |
|---|---|
| TSO primer: | N/A |
| AAGCAGTGGTATCAACGCAGAGTACATrGrG+G (SEQ ID | |
| NO: 1) | |
| ISPCR: | N/A |
| AAGCAGTGGTATCAACGCAGAGT (SEQ ID NO: 2) | |
| 3′-P2: | N/A |
| GTGACTGGAGTTCAGACGTGTGCTCTTCCGATC (SEQ ID | |
| NO: 3) | |
| short universal primer: | N/A |
| AATGATACGGCGACCACCGAGATCTACACTCTTTCCCTA | |
| CACGAC (SEQ ID NO: 4) | |
| illunima-QP2: | N/A |
| CAAGCAGAAGACGGCATACGA (SEQ ID NO: 5) | |
| POU5F1: | N/A |
| Forword: CAAACTGAGGTGCCTGCCCTTC (SEQ ID | |
| NO: 6) | |
| Reverse: ATTGAACTTCACCTTCCCTCCAACC (SEQ ID | |
| NO: 7) | |
| NANOG: | N/A |
| Forword: CATCTGCTGAGACCCTCGAC (SEQ ID NO: 8) | |
| Reverse: GGGCTTGTGGAAGAATCAGG (SEQ ID NO: 9) | |
| SOX2: | N/A |
| Forword: CATCAACGGTACACTGCCTCTC (SEQ ID | |
| NO: 10) | |
| Reverse: ACTCTCCTCCCATTTCCCTCTTT (SEQ ID | |
| NO: 11) | |
| REX1 (ZFP42): | N/A |
| Forword: GGGATCACGTGTGTGCAGAA (SEQ ID NO: 12) | |
| Reverse: TTCCTGCGACAGCCTTCAAA (SEQ ID NO: 13) | |
| CDH1: | N/A |
| Forword: GACTTCTGCCAGAGGAACCC (SEQ ID NO: 14) | |
| Reverse: CACTGGCCCCATGTGTTAGT (SEQ ID NO: 15) | |
| IGF2: | N/A |
| Forword: CGTGCTGCTATGCTGCTTAC (SEQ ID NO: 16) | |
| Reverse: AAGCAGCACTCTTCCACGAT (SEQ ID NO: 17) | |
| SNAI2: | N/A |
| Forword: CCCACACCCTACCTTGTGTC (SEQ ID NO: 18) | |
| Reverse: TGACATCCGAGTGTGTCTGC (SEQ ID NO: 19) | |
| SRC: | N/A |
| Forword: GCCAACATCCTGGTTGGAGA (SEQ ID NO: 20) | |
| Reverse: ATCCCAAAGGACCACACGTC (SEQ ID NO: 21) | |
| WNT5A: | N/A |
| Forword: CGCGAAGACAGGCATCAAAG (SEQ ID NO: 22) | |
| Reverse: CCTATCTGCATGACCCTGCC (SEQ ID NO: 23) | |
| GATA6: | N/A |
| Forword: CACTACTTGTGCAACCGCTG (SEQ ID NO: 24) | |
| Reverse: TTCTGCGGCTTTATGAGGGG (SEQ ID NO: 25) | |
| HAND1: | N/A |
| Forword: TGCGAGTGCATACCTTCTGT (SEQ ID NO: 26) | |
| Reverse: AGGCCCTGAGGGGAGTTTAT (SEQ ID NO: 27) | |
| PAX6: | N/A |
| Forword: TGTCCAACGGATGTGTGAGT (SEQ ID NO: 28) | |
| Reverse: TCTGTCTCGGATTTCCCAA (SEQ ID NO: 29) | |
| CDH2: | N/A |
| Forword: GCCTCAAGCCAACCTTACCT (SEQ ID NO: 30) | |
| Reverse: AGCTCTTGAGGAAAAGGCCC (SEQ ID NO: 31) | |
| ID2: | N/A |
| Forword: TCGCACCCCACTATTGTCAG (SEQ ID NO: 32) | |
| Reverse: TTCAGAAGCCTGCAAGGACA (SEQ ID NO: 33) | |
| ID3: | N/A |
| Forword: CATCTTCCCATCCAGACAGCC (SEQ ID | |
| NO: 34) | |
| Reverse: GTCAAGTGGGCACGACAAAG (SEQ ID NO: 35) | |
| BMP2: | N/A |
| Forword: GTCTTTCGGGAGCAGACACA (SEQ ID NO: 36) | |
| Reverse: GTCACCAACCTGGTGTCCAA (SEQ ID NO: 37) | |
| EOMES: | N/A |
| Forword: ACTCCCATGGACCTCCAGAA (SEQ ID NO: 38) | |
| Reverse: TCGCTTACAAGCACTGGTGT (SEQ ID NO: 39) | |
| T: | N/A |
| Forword: GAAGTACGTGAACGGGGAGT (SEQ ID NO: 40) | |
| Reverse: CACGATGTGGATTCGAGGCT (SEQ ID NO: 41) | |
| LIN28A: | N/A |
| Forword: TGCCGGCATCTGTAAATGGT (SEQ ID NO: 42) | |
| Reverse: ACTCTGGTGCACAAAGACGT (SEQ ID NO: 43) | |
| C-MYC: | N/A |
| Forword: ATCCAAGACCACCACCACTG (SEQ ID NO: 44) | |
| Reverse: GTTCACAGCAACATTCAGGTAGA (SEQ ID | |
| NO: 45) | |
| ETV4: | N/A |
| Forword: AGAACCGGCCAGCTATGAAC (SEQ ID NO: 46) | |
| Reverse: ATTGTCCGGGAAAGCCAGAG (SEQ ID NO: 47) | |
| ETV5: | N/A |
| Forword: TCAGCACATGGGTTCCAGTC (SEQ ID NO: 48) | |
| Reverse: CCTTCATGGCTGCTGGAGAA (SEQ ID NO: 49) | |
| BMP4: | N/A |
| Forword: TTCATTTTAGGAGCCATTCTGTAGT (SEQ ID | |
| NO: 50) | |
| Reverse: TCCTAGCAGGACTTGGCATAAT (SEQ ID | |
| NO: 51) | |
| EF-1α: | N/A |
| Forword: AATGCGGTGGGATCGACAAA (SEQ ID NO: 52) | |
| Reverse: CACGCTCACGTTCAGCCTTT (SEQ ID NO: 53) | |
| TYR gene sgRNA targeting sequence: | N/A |
| Forword: ACCGACCTCAGTTCCCCTTCACCG (SEQ ID | |
| NO: 54) | |
| Reverse: AAACCGGTGAAGGGGAACTGAGGT (SEQ ID | |
| NO: 55) | |
| TYR gene mutant site test primer: | N/A |
| Forword: CTGGACTTTCCAGACTTCCG (SEQ ID NO: 56) | |
| Reverse: GTGCAGTTGGGTCCCTGAAA (SEQ ID NO: 57) | |
| GFP test primer | N/A |
| Forword: CAACACCCGCATCGAGAAGT (SEQ ID NO: 58) | |
| Reverse: ACCACGAAGCTGTAGTAGCC (SEQ ID NO: 59) | |
| NANOG knock-in 3′ ARM test primer | N/A |
| Forword: GCAACCTCCCCTTCTACGAG (SEQ ID NO: 60) | |
| Reverse: GTGAAGCACACGGTAGGGTA (SEQ ID NO: 61) | |
| NANOG knock-in 5′ ARM test primer | N/A |
| Forword: TGTCCATTGCTGAAGCATGTAAT (SEQ ID | |
| NO: 62) | |
| Reverse: GAAGTTAGTAGCTCCGCTTCCTG (SEQ ID | |
| NO: 63) | |

### Example 1: Establishment of Pluripotent Stem Cell Lines from Pig Embryo Epiblasts

Epiblast from pig E8-E10 embryos was obtained, and the epiblast was treated with TrypLE™ Express followed by repeatedly blowing to disperse into small cell masses. The cell masses were then inoculated into 12-well cell culture dishes (density of the feeder layer cells were approximately 2×10⁵ cells/well) supplemented with 750 μL of 3i/LAF medium (Note: 10 μM Y27632 was added into the 3i/LAF medium), wherein the feeder layer cells were mouse embryonic fibroblasts treated with mitomycin C (Selleckchem, S8146) and cultured in an incubator at 37° C., 100% humidity, and 5% CO₂. After 12 hrs, another 750 μL of 3i/LAF medium was added without changing the medium, and after 24 hrs, the medium was replaced with fresh 3i/LAF medium (containing 2 μM Y27632). After that, the medium was replaced with fresh medium every 12 hrs, and after the clones grew for 2-3 days, the morphology of the clones was observed and the clones were digested for passaging.

The 3i/LAF medium contains basal medium (BM) as well as CHIR99021 (1 μM, Selleckchem, S1263), IWR-1-endo (2.5 μM, Selleckchem, S7086), WH-4-023 (1 μM, Selleckchem, S7565), recombinant human LIF (10 ng/mL, PeproTech, 300-05), recombinant human Activin A (25 ng/Ml, Peprotech, 120-14E), and recombinant human FGF-basic (10 ng/mL, Peprotech, 100-18B); wherein the basal medium (BM) contains 227.5 mL DMEM/F12 (Thermo Fisher 729 Scientific, 10565-018), 227.5 mL Neurobasal (Thermo Fisher Scientific, 21103-049), 2.5 mL N₂ supplement (Thermo Fisher Scientific, 17502-048), 5 mL B27 supplement (Thermo Fisher Scientific, 12587-010), 0.5% GlutaMAX (Thermo Fisher Scientific, 35050-061), 1% Non-essential amino acids (Thermo Fisher 732 Scientific, 11140-050), 0.1 mM β-mercaptoethanol (Thermo Fisher Scientific, 21985-023), 1% penicillin-streptomycin (Thermo FisherScientific, 15140-122), 5% knockout serum replacement (KOSR, Thermo Fisher Scientific, A3181502, optional), and 50 μg/mL ascorbic acid (Sigma-Aldrich, A4544).

Accutase Cell Dissociation Reagent (Gibco, A11105-01) was used for passage, the cells were digested in an incubator at 37° C. for 3-5 mins. An equal amount of medium was added to terminate the digestion when the majority of the clones were detached. The cells were gently blown for 10-15 times to make the cell clones well dispersed, and then inoculated according to the passage ratio of 1:3-1:5, and 10 μM of Y27632 was added into the 3i/LAF medium used for passage.

The cell lines obtained by the above method are called pre-gastrulation epiblast stem cell lines (pgEpiSCs). The pgEpiSCs could be established from Epiblast of all pig E8-E10 embryos. The morphological characteristics of cell lines established from Epiblast of embryos at different embryonic stages are shown in FIG. 1A, and the growth efficiency and cell line establishment efficiency of outgrowth cells at different embryonic stages are shown in FIG. 1B. In addition, for the convenience of writing, the pgEpiSCs described in Examples 3-6 below refer to cell lines established from E10 Epiblast.

### Example 2: Effect of Different Culture Conditions on the Preparation of pgEpiSCs

By analyzing the quality-controlled retained scRNA-seq data from a total of 1,458 single cells sampled from E1 to E14 oocytes and embryos of pig, the inventors have found that gastrulation should be blocked by the use of small molecule inhibitors associated with WNT signaling, as well as by the use of the cytokines from TGF-β superfamily and FGF family to promote Epiblast self-renewal, thus promoting the establishment of pgEpiSCs, and finally, a culture condition consisting of 3 WNT signaling pathway related inhibitors (CHIR99021, IWR-1-ENDO and WH-4-023) and 3 cytokines (LIF, Activin A and FGF2), i.e., the 3i/LAF medium as described in Example 1.

According to the 3i/LAF medium and its culture method described in Example 1, the inventors tested the requirement for each factor in the 3i/LAF medium for long-term in vitro maintenance of pgEpiSCs on the basis of removing the small molecule inhibitors and cytokines from the culture medium one by one without altering the other conditions.

The inventors found that removal of any of the three WNT signaling pathway-related inhibitors disrupted the desired domed clone morphology and weakened alkaline phosphatase (AP) staining signaling intensity (FIG. 2-1A); this also up-regulated epithelial-mesenchymal transition (EMT)-related genes, including IGF2, SNAI2, SRC, and WNT5A (FIG. 2-1B). In particular, removal of IWR-1-endo directly resulted in poorly defined boundaries of the pgEpiSC clone and led to significant down-regulation of core pluripotency factors such as NANOG, POU5F1, SOX2 and REX1 (FIG. 2-1B).

In addition, removal of IWR-1-endo or WH-4-023 may result in mesodermal and endodermal differentiation of pgEpiSCs as evidenced by the up-regulation of gastrulation marker genes BMP2, BMP4, EOMES, and T (FIG. 2-1B) and led to reduced accumulation or heterogeneity of the pluripotency factor POU5F1, while promoting the expression of mesodermal and endodermal progenitor marker EOMES (FIG. 2-1C). CHIR99021 had a dual role in the maintenance of pluripotency in mouse and human PSCs, with low concentration promoting self-renewal and high concentration promoting differentiation. Removal of CHIR99021 down-regulated the expression of cell proliferation-related genes such as LIN28A, C-MYC, ETV4 and ETV5 (FIG. 2-1D), suggesting that pgEpiSC proliferation was impaired. High concentration of CHIR99021 resulted in down-regulation of pluripotency marker POU5F1 and significant up-regulation of endodermal marker GATA6 (FIG. 2-1E), which were further confirmed by immunofluorescence staining (FIG. 2-1F), suggesting that CHIR99021 was conserved in mouse, human and pig PSCs.

After the removal of cytokines in 3i/LAF medium, the removal of the TGF-β superfamily member Activin A was detected, which led to the decrease of the level of pluripotency marker NANOG (FIG. 2-1G-FIG. 2-1H). Further supporting the role of Activin A in the long-term culture of pgEpiSCs, the addition of the TGF-β universal inhibitor, SB431542, into the culture medium (to avoid the effect of the feeder secretion) resulted in irregular clone morphology and a dramatic decrease in the level of NANOG (FIG. 2-1G-FIG. 2-1H). Notably, the addition of SB431542 also resulted in a significant decrease in pluripotency markers (e.g., POU5F1 and REX1) and a significant increase in BMP4 and BMP downstream transcription factors (e.g., ID2 and ID3), which mediate the induction of primitive streak during embryonic development (Kurek et al., 2015; Valdez Magana et al. 2014) (FIG. 2-1H).

When FGF2 was removed (and the ERK/MEK inhibitor PD0325901 was added), pgEpiSCs could not proliferate or be passaged normally (FIG. 2-1I-FIG. 2-1K). The inventors also noted that decreasing the concentration of FGF2 significantly reduced the proliferative capacity of pgEpiSCs when all other conditions remained unchanged (FIG. 2-1K), and that low concentrations of FGF2 were detrimental to cell proliferation, whereas high concentrations resulted in faster cell proliferation. Notably, the inventors found that LIF was not necessary to maintain the morphology of pgEpiSC clones (FIG. 2-1L), but the addition of the JAK1/2 inhibitor, ruxolitinib, flattened the clones, and western blotting showed that phosphorylated STAT3 could be detected only in the presence of LIF (FIG. 2-1M), suggesting that pgEpiSCs can respond to the pluripotency-promoting effects of LIF.

In addition, according to the 3i/LAF medium and its culture method described in Example 1, the inventors performed the following tests on the basis of other conditions unchanged.
(1) Only IWR-1-endo was replaced with XAV939 while other conditions remain unchanged.

After IWR-1-endo being replaced by XAV939, the cells were unable to maintain pluripotency for a long period of time, and AP positivity was lost at about 8 generations of passaging (as shown in FIG. 2-2A).

After IWR-1-endo being replaced by XAV939, the expression of core pluripotency factor OCT4 (POU5F1) was down-regulated; and the expression of pluripotency factors REX1, STELLA, and ESRRB was down-regulated (as shown in FIG. 2-2B).
(2) Only the concentration of CHIR99021 was adjusted while other conditions remain unchanged.

The concentration of CHIR99021 in the culture system affects the pluripotency and homogeneity of cells. When the concentration of CHIR99021 was high, the expression of pluripotency gene POU5F1 was decreased, and the expression of gene for lineage specification-associated gene GATA6 was up-regulated, showing a heterogeneous expression pattern. The expression of mesoderm marker gene TBX3 and endoderm gene ESRRB increased with the concentration of CHIR99021 (as shown in FIG. 2-2C and FIG. 2-2D).
(3) Only the concentration ratio of CHIR99021 to IWR-1-endo was adjusted, while other conditions remain unchanged.

The concentration ratio of CHIR99021 to IWR-1-endo has an effect on pluripotency, the optimal ratio of C/I≈1:2-1:3 shows the highest expression of the pluripotency factor Nanog (as shown in FIG. 2-2E).

According to the 3i/LAF medium and its culture method configured in Example 1, the inventors also investigated the effect of the adjusted or replaced medium on the long-term in vitro maintenance of pgEpiSCs on the basis of adjusting the concentrations of the small molecule inhibitors and cytokines or replacing the compositions of the small molecule inhibitors and cytokines in the culture medium one by one without altering the other conditions.

(1) Adjusting the Concentration of Small Molecule Inhibitors and Cytokines in 3i/LAF Medium

Medium adjustment: compared with the 3i/LAF medium and its culture method configured in Example 1 above, only the concentrations of small molecule inhibitors or cytokines in the 3i/LAF medium were single-factor adjusted as shown in Table A below, respectively, and the other conditions were kept unchanged.

Experimental method: pgEpiSCs were cultured with the adjusted medium, after which AP staining was performed on the cultured pgEpiSCs of P1 (indicating the pgEpiSCs after 1 passage in the adjusted medium) or P3 (indicating the pgEpiSCs after 3 passages in the adjusted medium). The specific steps of AP staining are described in the section of Experimental Methods described previously.

Principle of the experiment: undifferentiated stem cells will express Ap at high level. By staining fixed stem cells, differentiated cells show no color and undifferentiated cells show purple or red color.

Experimental result: the results are shown in Table A or FIGS. 2-3A to 2-3F below.

TABLE A
Adjustment of the concentrations of small molecule inhibitors or
cytokines in 3i/LAF medium and test results

| Group | | | | |
|---|---|---|---|---|
| No | | | | |
| of the | | Small molecule | | |
| adjusted | Passage of | inhibitors | Concen- | Test results on |
| medium | the pgEpiSCs | or cytokines | tration | pluripotency |
| 1 | P1 | CHIR99021 | 0.01 μM | As shown in FIG. |
| | | | 1 μM | 2-3A, left panel |
| | | | 3 μM | |
| | P3 | CHIR99021 | 0.01 μM | As shown in FIG. |
| | | | 1 μM | 2-3A, right panel |
| | | | 3 μM | |
| 2 | P1 | IWR-1-endo | 1 μM | As shown in FIG. |
| | | | 2.5 μM | 2-3B, left panel |
| | | | 3 μM | |
| | P3 | IWR-1-endo | 1 μM | As shown in FIG. |
| | | | 2.5 μM | 2-3B, right panel |
| | | | 3 μM | |
| 3 | P1 | WH-4-023 | 0.01 μM | As shown in FIG. |
| | | | 1 μM | 2-3C, left panel |
| | | | 5 μM | |
| | P3 | WH-4-023 | 0.01 μM | As shown in FIG. |
| | | | 1 μM | 2-3C, right panel |
| | | | 5 μM | |
| 4 | P1 | Recombinant | 0.01 | As shown in FIG. |
| | | human | ng/mL | 2-3D, left panel |
| | | Activin A | 25 ng/mL | |
| | | | 100 | |
| | | | ng/mL | |
| | P3 | Recombinant | 0.01 | As shown in FIG. |
| | | human | ng/mL | 2-3D, right panel |
| | | Activin A | 25 ng/mL | |
| | | | 100 | |
| | | | ng/mL | |
| 5 | P1 | Recombinant | 1 ng/mL | As shown in FIG. |
| | | human | 25 ng/mL | 2-3E, left panel |
| | | FGF-basic | 100 | |
| | | | ng/mL | |
| | P3 | Recombinant | 1 ng/mL | As shown in FIG. |
| | | human | 25 ng/mL | 2-3E, right panel |
| | | FGF-basic | 100 | |
| | | | ng/mL | |
| 6 | P1 | Recombinant | 1 ng/mL | As shown in FIG. |
| | | human LIF | 25 ng/mL | 2-3F, left panel |
| | | | 100 | |
| | | | ng/mL | |
| | P3 | Recombinant | 1 ng/mL | As shown in FIG. |
| | | human LIF | 25 ng/mL | 2-3F, right panel |
| | | | 100 | |
| | | | ng/mL | |

It can be seen from FIGS. 2-3A˜2-3F that the medium obtained by adjusting the concentration of small molecular inhibitors or cytokines in 3i/LAF medium can still maintain the pluripotency of pgEpiSCs in vitro for a long time.

### (2) Replacing the Small Molecular Inhibitors and Cytokines Components in 31/LAF Medium

Medium adjustment: compared with the 3i/LAF medium and its culture method configured in Example 1 above, only the small molecular inhibitors or cytokines components in the 3i/LAF medium were replaced by single factor as shown in Table B below, and other conditions remain unchanged.

Experimental method: pgEpiSCs were cultured with the adjusted medium, after which AP staining was performed on the cultured pgEpiSCs of P1 (indicating the pgEpiSCs after 1 passage in the adjusted medium) or P3 (indicating the pgEpiSCs after 3 passages in the adjusted medium). The specific steps of AP staining are described in the section of Experimental Methods described previously.

Principle of the experiment: undifferentiated stem cells will express Ap at high level. By staining fixed stem cells, differentiated cells show no color and undifferentiated cells show purple or red color.

Experimental result: the results are shown in Table B or FIGS. 2-3G to 2-3I below.

TABLE B
Replacement of small molecular inhibitors or cytokines components in
3i/LAF medium and test results

| Group | | | | |
|---|---|---|---|---|
| No. | | | | |
| of the | Passage | | | Test results |
| adjusted | of the | Small molecule | Con- | on |
| medium | pgEpiSCs | inhibitors or cytokines | centration | pluripotency |
| 7 | P1 | CHIR99021 was | 0.1 ng/mL | As shown in |
| | | replaced with WNT3a | (i.e. 0.0025 | FIG. 2-3G, |
| | | | nM) | left panel |
| | | | 1 ng/mL | |
| | | | (i.e. 0.025 | |
| | | | nM) | |
| | | | 10 ng/mL | |
| | | | (i.e. 0.25 | |
| | | | nM) | |
| | P3 | CHIR99021 was | 0.1 ng/mL | As shown in |
| | | replaced with WNT3a | (i.e. 0.0025 | FIG. 2-3G, |
| | | | nM) | left panel |
| | | | 1 ng/mL | |
| | | | (i.e. 0.025 | |
| | | | nM) | |
| | | | 10 ng/mL | |
| | | | (i.e. 0.25 | |
| | | | nM) | |
| 8 | P1 | WH-4-023 was re- | 3 nM | As shown in |
| | | placed with A419259 | 300 nM | FIG. 2-3H, |
| | | Recombinant human | 30 μM | left panel |
| | P3 | Activin A was replaced | 3 nM | As shown in |
| | | with Nodal [symbol] placed | 300 nM | FIG. 2-3H, |
| | | with A419259 | 30 μM As shown in [symbol] el | |
| | | Recombinant human | FIG. 2-3I, right panel | |
| 9 | P1 | Activin A was replaced | 0.1 ng/mL | [symbol] n in |
| | | with Nodal [symbol] man | 1 ng/mL | FIG. 2-3I, |
| | | Activin A was | 10 ng/mL | left panel |
| | | replaced with Nodal | | |
| | P3 | Recombinant human | 0.1 ng/mL | As shown in |
| | | Activin A was | 1 ng/mL | FIG. 2-3I, |
| | | replaced with Nodal | 10 ng/mL | right panel |

[symbol]

indicates data missing or illegible when filed

It can be seen from FIG. 2-3G˜2-3I that the medium obtained by replacing the small molecular inhibitors or cytokines components in 3i/LAF medium can still maintain the pluripotency of pgEpiSCs in vitro for a long time.

### Example 3: Characterization of pgEpiSC

pgEpiSCs from E10 epiblast could be passaged at the single-cell level by enzymolysis at passage ratios of 1:3-1:5/2˜3 days. The doubling time for pgEpiSC proliferation was about 16 hours (FIGS. 3A-3B), and the efficiency of single-cell colony formation was about 33.83% (FIG. 3C).

It was found that pgEpiSCs retained their dome-shaped clonal morphology (FIG. 3D), and were AP-positive (FIG. 3E). pgEpiSCs expressed pluripotent stem cell markers such as POU5F1, NANOG, and SOX2 (FIG. 3F), as well as pluripotent surface markers, including SSEA1, SSEA4, TRA-1-81, and TRA-1-60 (FIG. 3G), in long-term in vitro culture.

In addition, the embryonic body (EB) differentiation assay showed that pgEpiSCs could differentiate into three germ layers upon removal of the inhibitors and cytokines from the medium (FIG. 3H). Directional induced differentiation assays showed that pgEpiSCs could also differentiate into the expected three germ layers in conditioned medium (FIG. 3I). Teratoma formation assays confirmed that pgEpiSCs developed into the expected three germ layers in vivo (FIG. 3J).

All pgEpiSC lines could be passaged over 60 times without changing the above characteristics. By comparing the karyotypes, SNVs and Short InDels (≤30 bp) of pgEpiSC cell lines between different passages of the same cell line and between different donor cell lines, the inventors found that the cell lines had normal karyotypes and very few mutations were detected after 60 passages in culture (FIG. 3K). Then two cell lines were randomly selected for long-term maintenance capability testing, which had been passaged for at least 216 times.

These results demonstrate the successful establishment of stable pluripotent stem cell lines from pig pre-gastrulation epiblasts.

### Example 4: Correlation Between the Transcriptome of pgEpiSC and Pre-Gastrulation Epiblast Cells

To investigate the transcriptome characteristics of pgEpiSCs, scRNA-seq was performed on pgEpiSCs from passage 10 and passage 60 (i.e., low- and high-passages), and then the transcriptome of pgEpiSCs was compared to that of a single cell from pig embryo (from E0 to E14). tSNE visualization showed that the pgEpiSCs clustered in a group independent of embryonic cells at all periods (from E0 to E14) (FIG. 4A). Gene expression levels of lineage specification marker genes showed that Epiblast-specific genes (NANOG, TDGF1, ETV4, GDF3, and NODAL, etc.) were highly and uniformly expressed in pgEpiSCs (FIG. 4B), suggesting that pgEpiSCs maintain the transcriptomic properties of Epiblast cells. Principal component analysis (PCA) of DEGs relevant with pluripotency and differentiation revealed that pgEpiSCs clustered closely to E10 epiblast cells (FIG. 4C). Pairwise correlation analysis demonstrated that low- and high-passage pgEpiSCs exhibited remarkable consistency (r=0.97, P<2.2×10⁻¹⁶, Spearman's rank correlation), and collectively showed the greatest similarity to the E10 Epiblast compared to epiblast or ectoderm cells from other embryonic stages (mean r=0.88, P<2.2×10⁻¹⁶, Spearman's rank correlation) (FIG. 4D). In addition, the expression levels of classical pluripotency and gastrulation marker genes in pgEpiSCs indicated that they were closest to those of the E10 Epiblast (FIG. 4E). All these results suggest that pgEpiSCs have similar transcriptomic characteristics as their origin E10 Epiblast.

Next, comparative transcriptome analysis of the RNA-seq data of pgEpiSCs with human naïve, conventional, and formative PSCs, as well as with mouse naïve, primed, and previously reported formative PSCs were performed (Guo et al., 2017; Ji et al., 2016; Kinoshita et al., 2021). It was found that pgEpiSCs were more similar to formative and primed (or conventional) PSCs than naïve PSCs (FIG. 4F). Importantly, pgEpiSCs exhibited stronger formative hPSC-specific gene expression compared to conventional and naïve hPSCs (FIG. 4G). Representative genes with increased expression are shown in Table 4-1, and representative genes with reduced expression are shown in Table 4-2 compared to conventional hESCs.

TABLE 4-1
Representative genes with increased expression compared to
conventional hESC

| | | Expression | pgEpiSC/ |
|---|---|---|---|
| | Expression level in | level in | conventional |
| Gene | conventional hESC | pgEpiSC | hESC |
| ADPRM | 2.66 | 6.21 | 2.33 |
| FRG1 | 2.51 | 5.23 | 2.08 |
| GAS2 | 2.05 | 5.49 | 2.68 |
| HK3 | 0.15 | 5.13 | 35.23 |
| NCAN | 2.65 | 6.16 | 2.32 |
| POUSF1B | 1.29 | 10.62 | 8.25 |
| ZFP2 | 1.43 | 6.76 | 4.74 |
| CLDND2 | 0.83 | 5.56 | 6.68 |
| CRK | 2.43 | 9.58 | 3.94 |
| DMP1 | 0.23 | 6.26 | 27.42 |
| GATD3B | 0.95 | 5.31 | 5.62 |
| H3F3A | 3.55 | 8.48 | 2.39 |
| IRF8 | 0.69 | 8.06 | 11.72 |
| ITGA4 | 2.47 | 5.26 | 2.13 |
| KRT14 | 0.08 | 7.65 | 90.77 |
| MPC1 | 3.04 | 6.38 | 2.10 |
| MSH4 | 0.64 | 6.83 | 10.60 |
| NDE1 | 1.90 | 6.36 | 3.35 |
| PBX2 | 3.24 | 6.54 | 2.02 |
| PRKY | 2.17 | 6.02 | 2.77 |
| RGL2 | 3.26 | 7.78 | 2.39 |
| SOX10 | 2.35 | 5.07 | 2.15 |
| VHLL | 0.03 | 5.19 | 178.33 |

TABLE 4-2
Representative genes with reduced expression compared to
conventional hESC

| | Expression level in | Expression level in | pgEpiSC/ |
|---|---|---|---|
| | conventional hESC | pgEpiSC | conventional |
| Gene | [symbol] | [symbol] | hESC |
| ABCC4 | 5.18 | 0.38 | 0.07 |
| ADCY2 | 6.03 | 2.02 | 0.33 |
| AK2 | 6.29 | 0.21 | 0.03 |
| AKT1 | 6.25 | 0.01 | 0.00 |
| BMP2 | 5.10 | 1.40 | 0.27 |
| CD46 | 7.49 | 0.08 | 0.01 |
| CDH3 | 7.54 | 2.84 | 0.38 |
| DNM1 | 5.09 | 1.98 | 0.39 |
| DPPA4 | 9.26 | 0.81 | 0.09 |
| ETS1 | 5.99 | 1.99 | 0.33 |
| GAB2 | 5.11 | 2.06 | 0.40 |
| ID2 | 5.51 | 1.56 | 0.28 |
| KDR | 6.81 | 0.26 | 0.04 |
| MMP24 | 5.72 | 1.38 | 0.24 |
| TGFB1 | 5.64 | 2.52 | 0.45 |
| VGLL3 | 5.02 | 1.55 | 0.31 |
| ZNF195 | 6.86 | 0.13 | 0.02 |
| ZNF519 | 5.36 | 0.13 | 0.02 |

These results support the successful establishment of PGEPICS cell line, and also show that PGEPICS has the characteristics and pluripotency of E10 pre-embryonic epithelial cells.

### Example 5 Spatial Regulation Characteristics of pgEpiSC Transcription

Using the high-deep in situ high-throughput chromatin conformation capture (hic) sequencing, the three-dimensional genome structure of pgEpiSCs and pig embryonic fibroblasts (pEFs) were reconstructed, and ultimately obtained the maps with the maximum resolution of 300 bp after combining the data from 16 replicates. It was found that the chromatin spatial plasticity of pgEpiSCs is higher than that of pEFs (reflected by the reduced extent of chromosome intermingling in the pgEpiSCs compared to the pEFs: 0.18/0.71, P<2.2×10⁻¹⁶, Wilcoxon rank-sum test) (FIGS. 5A-5B). The more disordered chromatin in the pgEpiSCs was also evident based on its high entropy status (1.57/1.00, P=5.63×10⁻⁴, Wilcoxon rank-sum test) (FIGS. 5C-5D), which is consistent with previous studies on humans and mice (Lindsly et al., 2021; Tan et al., 2018). It can be seen that the typical loose regulatory architecture we detected in high-deep in-situ Hi-C analysis may be due to the pluripotency of pgEpiSCs.

Furthermore, we studied that the relatively loose heterochromatin regulatory structure of pgEpiSCs may affect pluripotency. It was found that compared with PEF, pgEpiSCs has less promoter-enhancer interactions (PEIs) (pgEpiSCs, 20,389 enhancers are allocated to 6,498 promoters) (PEF, 30,852 enhancers are allocated to 7,823 promoters). There are obvious transcriptome differences between pgEpiSCs and pEFs, and only 5,547 PEIs are shared. The regulatory potential score (RPS) for each promoter was calculated, which is an index based on spatial proximity and represents the joint regulatory effect of multiple enhancers on a given gene (Cao et al., 2017; Fulco et al., 2019; Whalen et al., 2016), for a given promoter, itse RPS was calculated as: Σₙ (log 10In), wherein In is the normalized interaction intensity of PEI n of the promoter. A total of 875 co-variant genes of RPS and gene expression were identified (that is, the genes with higher RPS score in pgEpiSCs were usually up-regulated compared with pEFs (log 2 fold change [FC]>1, FDR<0.05)), among which 75 genes were strongly expressed in pgEpiSC's (TPM>5 compared with TPM<0.5 in pEFs), and representative co-variant genes strongly expressed in pgEpiSCs are exemplified in Table 5.

TABLE 5
Representative co-variant genes strongly expressed in pgEpiSCs

| | | | | Log₂ (fold |
|---|---|---|---|---|
| | | | TPM | change) |
| | Gene | TPM in | in | of expression |
| Ensembl ID | symbol | pEpiSCs | pEFs | (pEpiSCs/pEFs) |
| ENSSSCG00000003557 | LIN28A | 435.21 | 0.00 | 15.85 |
| ENSSSCG00000007252 | DNMT3B | 263.29 | 0.22 | 9.46 |
| ENSSSCG00000032299 | LEFTY2 | 193.44 | 0.02 | 12.85 |
| ENSSSCG00000035444 | ZFP42 | 174.32 | 0.09 | 10.69 |
| ENSSSCG00000038188 | | 124.69 | 0.06 | 10.66 |
| ENSSSCG00000033478 | | 106.46 | 0.00 | 11.88 |
| ENSSSCG00000000252 | KRT8 | 85.84 | 0.15 | 8.67 |
| ENSSSCG00000040403 | NANOG | 65.01 | 0.13 | 8.46 |
| ENSSSCG00000000253 | KRT18 | 58.26 | 0.03 | 10.32 |
| ENSSSCG00000008429 | EPCAM | 47.52 | 0.01 | 11.46 |
| ENSSSCG00000017934 | CLDN7 | 38.33 | 0.22 | 6.59 |
| ENSSSCG00000026894 | NFE2L3 | 26.68 | 0.20 | 6.33 |
| ENSSSCG00000025652 | CDH1 | 25.30 | 0.02 | 9.40 |
| ENSSSCG00000005063 | OTX2 | 24.00 | 0.00 | 13.32 |
| ENSSSCG00000004679 | SORD | 23.84 | 0.31 | 5.53 |
| ENSSSCG00000031204 | | 22.27 | 0.00 | 11.61 |
| ENSSSCG00000022739 | DSG2 | 19.28 | 0.35 | 5.10 |
| ENSSSCG00000028804 | CCDC181 | 18.11 | 0.12 | 6.67 |
| ENSSSCG00000021207 | HESX1 | 17.57 | 0.30 | 5.37 |
| ENSSSCG00000040222 | | 17.50 | 0.31 | 5.83 |
| ENSSSCG00000036181 | | 15.62 | 0.12 | 6.73 |
| ENSSSCG00000039049 | MAP7 | 15.44 | 0.02 | 9.32 |
| ENSSSCG00000021307 | USP44 | 14.65 | 0.03 | 8.29 |
| ENSSSCG00000007718 | CLDN4 | 13.89 | 0.03 | 7.70 |
| ENSSSCG00000012003 | | 13.64 | 0.33 | 4.51 |
| ENSSSCG00000010771 | UTF1 | 12.92 | 0.00 | 11.98 |
| ENSSSCG00000002361 | VRTN | 12.12 | 0.00 | 13.88 |
| ENSSSCG00000015246 | ST14 | 11.63 | 0.00 | 13.66 |
| ENSSSCG00000013469 | ZNF555 | 11.01 | 0.27 | 4.78 |
| ENSSSCG00000021767 | | 10.52 | 0.02 | 8.79 |
| ENSSSCG00000002884 | LSR | 10.38 | 0.01 | 9.72 |
| ENSSSCG00000006195 | PRDM14 | 9.95 | 0.00 | 12.47 |
| ENSSSCG00000008902 | PPAT | 9.92 | 0.24 | 4.66 |
| ENSSSCG00000017915 | VMO1 | 9.89 | 0.07 | 6.43 |
| ENSSSCG00000002456 | CHGA | 9.78 | 0.01 | 10.21 |
| ENSSSCG00000031347 | | 9.78 | 0.20 | 4.82 |
| ENSSSCG00000016841 | SLC1A3 | 9.72 | 0.11 | 5.65 |
| ENSSSCG00000000766 | CECR2 | 9.61 | 0.12 | 5.47 |
| ENSSSCG00000009429 | TNFSF11 | 9.32 | 0.38 | 3.54 |
| ENSSSCG00000002032 | SLC7A8 | 9.30 | 0.08 | 5.75 |
| ENSSSCG00000009120 | ZGRF1 | 9.16 | 0.42 | 3.94 |
| ENSSSCG00000008618 | MYCN | 8.95 | 0.02 | 8.01 |
| ENSSSCG00000008465 | KCNG3 | 8.88 | 0.01 | 9.49 |
| ENSSSCG00000023861 | PFAS | 8.77 | 0.20 | 4.63 |
| ENSSSCG00000032831 | BRI3BP | 8.68 | 0.12 | 5.48 |
| ENSSSCG00000006099 | ESRP1 | 8.57 | 0.02 | 7.84 |
| ENSSSCG00000021259 | CDA | 8.42 | 0.06 | 6.38 |
| ENSSSCG00000026718 | PLCH1 | 7.93 | 0.03 | 7.75 |
| ENSSSCG00000021941 | ZSCAN21 | 7.82 | 0.43 | 3.60 |
| ENSSSCG00000007479 | SALL4 | 7.81 | 0.01 | 9.34 |
| ENSSSCG00000000298 | PPP1R1A | 7.78 | 0.08 | 5.94 |
| ENSSSCG00000007602 | BAIAP2L1 | 7.75 | 0.11 | 5.11 |
| ENSSSCG00000026996 | ABCC4 | 7.75 | 0.46 | 3.10 |
| ENSSSCG00000037485 | SOX2 | 7.63 | 0.00 | 11.05 |
| ENSSSCG00000009859 | TESC | 7.61 | 0.46 | 3.28 |
| ENSSSCG00000039169 | CER1 | 7.42 | 0.00 | 12.34 |
| ENSSSCG00000031675 | | 7.41 | 0.06 | 6.35 |
| ENSSSCG00000006050 | | 7.22 | 0.12 | 4.89 |
| ENSSSCG00000009584 | SEMA4D | 7.11 | 0.26 | 3.81 |
| ENSSSCG00000037744 | ZNF483 | 7.04 | 0.31 | 3.77 |
| ENSSSCG00000037612 | LIN28B | 6.96 | 0.08 | 5.62 |
| ENSSSCG00000011610 | NUP21 | 6.85 | 0.00 | 10.70 |
| ENSSSCG00000038138 | TFEC | 6.84 | 0.11 | 5.51 |
| ENSSSCG00000016140 | FZD5 | 6.62 | 0.07 | 5.53 |
| ENSSSCG00000011257 | ACVR2B | 6.55 | 0.21 | 4.24 |
| ENSSSCG00000034904 | | 6.36 | 0.00 | 5.97 |
| ENSSSCG00000025838 | AP1M2 | 6.13 | 0.00 | 12.70 |
| ENSSSCG00000034478 | | 6.09 | 0.00 | 7.22 |
| ENSSSCG00000014450 | TCOF1 | 5.76 | 0.39 | 2.99 |
| ENSSSCG00000011277 | CCK | 5.67 | 0.02 | 10.23 |
| ENSSSCG00000004621 | MYO5C | 5.64 | 0.01 | 8.86 |
| ENSSSCG00000025672 | RAVER2 | 5.57 | 0.32 | 3.44 |
| ENSSSCG00000033197 | | 5.56 | 0.00 | 5.17 |
| ENSSSCG00000034739 | | 5.44 | 0.01 | 8.85 |
| ENSSSCG00000004318 | RRAGD | 5.35 | 0.06 | 5.83 |

In addition, a specific interaction of OTX2 with the enhancer in pgEpiSCs was detected at (as well as LIN28A, NANOG, PRDM14, SALL4, UTF1 and ZFP42), whereas the enhancer was absent in pEFs (FIGS. 5E-5K).

### Example 6 Consecutive Gene Editing of pgEpiSCs and Production of Cloned Piglets

At present, one of the major limitations of using pig somatic cell nuclear transfer is that somatic donor cells can usually only support a single round of genome editing (Yan et al., 2018). In order to test whether pgEpiSCs could withstand consecutive genome editing, we conducted experiments of various forms of genome manipulation (FIG. 6A).

First, pgEpiSCs were obtained that were stably transfected with GFP-nls reporter fragment, and the rate of GFP-positive cells was 21.27% by flow cytometry (FIG. 6B). Second, using these GFP-positive cells, we carried out CRISPR/cas9-mediated knock-in, specifically, we inserted the tdTomato reporter gene cassette into the NANOG locus, which was located at a position immediately preceding the native stop codon and called GFP-NANOG-tdTomato pgEpiSCs (GN-pgEpiSCs) (FIG. 6C). The GN-pgEpiSC clones were selected according to NANOG-tdTomato fluorescence, and then re-amplified in 3i/LAF medium (FIG. 6D). Consistent with the known status of NANOG (pluripotency marker), no tdTomato reporter fluorescence was detected after experimentally induced differentiation of pgEpiSCs edited after knock-in (FIG. 6E). For the third and final genome modification, we performed a c-t transition using cytosine base editors (CBEs) (Koblan et al., 2018; Komor et al., 2016) at the stop codon of the TYR locus, inducing an albinism known to be associated with pig coat color (Li et al., 2018; Xie et al., 2019). 99 clones were sequenced, of which 24.24% ( 24/99) were heterozygotes and 3.03% ( 3/99) were homozygotes (the C-T base editing in the background of GNT-pgEpiSCs is referred to as GNT-pgEpiSCs) (FIG. 6F). These results show that pgEpiSCs could tolerate continuous genome modification, including traditional transgene insertion, CRISPR/Cas9 precision knock-in and single base transition editing using CBEs.

Then, we conducted a nuclear transfer experiment, and exclusively used wild-type (WT) pgEpiSCs, GFP-pgEpiSCs and GNT-pgEpiSCs as nuclear donor cells to obtain cloned embryos, and further mixed-transplanted 200 WT pgEpiSCs, 203 GFP-pgEpiSCs cloned embryos and 660 GNT-pgEpiSCs cloned embryos (FIG. 6G). We finally obtained 1 WT pgEpiSCs cloned piglet, 1 GFP-pgEpiSCs cloned piglet and 3 GNT-pgEpiSCs cloned piglets (FIG. 6H). The cloning efficiency of gene-edited pgEpiSC was similar to that of wild-type pgEpiSC cells and comparable to that of fibroblasts (FIG. 6G). Importantly, GNT-pgEpiSCs cloned piglets showed the expected albino coat color phenotype (FIG. 6I). These results show that pgEpiSCs are able to withstand continuous multi-gene editing and have the potential to generate complex pig models.

Although specific embodiments of the present invention have been described in detail, it will be understood by those skilled in the art that various modifications and variations to the details may be made in the light of all the published teachings and that these changes are within the scope of the present invention. The full scope of the present invention is given by the appended claims and any equivalents thereof.

### REFERENCES

Alberio, R., Croxall, N., and Allegrucci, C. (2010). Pig epiblast stem cells depend on activin/nodal signaling for pluripotency and self-renewal. Stem Cells Dev. 19, 1627-1636.
Bao, S., Tang, F., Li, X., Hayashi, K., Gillich, A., Lao, K., and Surani, M. A. (2009). Epigenetic reversion of post implantation epiblast to pluripotent embryonic stem cells. Nature 461, 1292-1295.
Bonev, B., Mendelson Cohen, N., Szabo, Q., Fritsch, L., Papadopoulos, G. L., Lubling, Y., Xu, X., Lv, X., Hugnot, J. P., Tanay, A., et al. (2017). Multiscale 3D Genome Rewiring during Mouse Neural Development. Cell 171, 557-572 e524.
Boroviak, T., Loos, R., Bertone, P., Smith, A., and Nichols, J. (2014). The ability of inner-cell-mass cells to self renew as embryonic stem cells is acquired following epiblast specification. Nat. Cell Biol. 16, 516-528.
Brons, I. G., Smithers, L. E., Trotter, M. W., Rugg-Gunn, P., Sun, B., Chuva de Sousa Lopes, S. M., Howlett, S. K., Clarkson, A., Ahrlund-Richter, L., Pedersen, R. A., et al. (2007). Derivation of pluripotent epiblast stem cells from mammalian embryos. Nature 448, 191-195.
Cao, Q., Anyansi, C., Hu, X., Xu, L., Xiong, L., Tang, W., Mok, M. T. S., Cheng, C., Fan, X., Gerstein, M., et al. (2017). Reconstruction of enhancer-target networks in 935 samples of human primary cells, tissues and cell lines. Nat. Genet. 49, 1428-1436.
Choi, K. H., Lee, D. K., Kim, S. W., Woo, S. H., Kim, D. Y., and Lee, C. K. (2019). Chemically Defined Media Can Maintain Pig Pluripotency Network In Vitro. Stem Cell Reports 13, 221-234.
Consortium, E. P. (2012). An integrated encyclopedia of DNA elements in the human genome. Nature 489, 57-74.
Colognori, D., Sunwoo, H., Kriz, A. J., Wang, C. Y., and Lee, J. T. (2019). Xist Deletional Analysis Reveals an Interdependency between Xist RNA and Polycomb Complexes for Spreading along the Inactive X. Mol. Cell 74, 101-117 e110.
Di Giammartino, D. C., Kloetgen, A., Polyzos, A., Liu, Y., Kim, D., Murphy, D., Abuhashem, A., Cavaliere, P., Aronson, B., Shah, V., et al. (2019). KLF4 is involved in the organization and regulation of pluripotency associated three-dimensional enhancer networks. Nat. Cell Biol. 21, 1179-1190.
Dixon, J. R., Jung, I., Selvaraj, S., Shen, Y., Antosiewicz-Bourget, J. E., Lee, A. Y., Ye, Z., Kim, A., Rajagopal, N., Xie, W., et al. (2015). Chromatin architecture reorganization during stem cell differentiation. Nature 518, 331-336.
Evans, M. J., and Kaufman, M. H. (1981). Establishment in culture of pluripotential cells from mouse embryos. Nature 292, 154-156.
Fulco, C. P., Nasser, J., Jones, T. R., Munson, G., Bergman, D. T., Subramanian, V., Grossman, S. R., Anyoha, R., Doughty, B. R., Patwardhan, T. A., et al. (2019). Activity-by-contact model of enhancer-promoter regulation from thousands of CRISPR perturbations. Nat. Genet. 51, 1664-1669.
Gafni, O., Weinberger, L., Mansour, A. A., Manor, Y. S., Chomsky, E., Ben-Yosef, D., Kalma, Y., Viukov, S., Maza, I., Zviran, A., et al. (2013). Derivation of novel human ground state naive pluripotent stem cells. Nature 504, 282-286.
Gao, X., Nowak-Imialek, M., Chen, X., Chen, D., Herrmann, D., Ruan, D., Chen, A. C. H., Eckersley-Maslin, M. A., Ahmad, S., Lee, Y. L., et al. (2019). Establishment of porcine and human expanded potential stem cells. Nat. Cell Biol. 21, 687-699.
Gao, S., Yan, L., Wang, R., Li, J., Yong, J., Zhou, X., Wei, Y., Wu, X., Wang, X., Fan, X., et al. (2018). Tracing the temporal-spatial transcriptome landscapes of the human fetal digestive tract using single-cell RNA sequencing. Nat. Cell Biol. 20, 721-734.
Guo, G., von Meyenn, F., Rostovskaya, M., Clarke, J., Dietmann, S., Baker, D., Sahakyan, A., Myers, S., Bertone, P., Reik, W., et al. (2017). Epigenetic resetting of human pluripotency. Development 144, 2748-2763.
Haraguchi, S., Kikuchi, K., Nakai, M., and Tokunaga, T. (2012). Establishment of self-renewing porcine embryonic stem cell-like cells by signal inhibition. J. Reprod. Dev. 58, 707-716.
Hou, D. R., Jin, Y., Nie, X. W., Zhang, M. L., Ta, N., Zhao, L. H., Yang, N., Chen, Y., Wu, Z. Q., Jiang, H. B., et al. (2016). Derivation of Porcine Embryonic Stem-Like Cells from In Vitro-Produced Blastocyst-Stage Embryos. Sci. Rep. 6, 25838.
Ji, X., Dadon, D. B., Powell, B. E., Fan, Z. P., Borges-Rivera, D., Shachar, S., Weintraub, A. S., Hnisz, D., Pegoraro, G., Lee, T. I., et al. (2016). 3D Chromosome Regulatory Landscape of Human Pluripotent Cells. Cell Stem Cell 18, 262-275.
Kinoshita, M., Barber, M., Mansfield, W., Cui, Y., Spindlow, D., Stirparo, G. G., Dietmann, S., Nichols, J., and Smith, A. (2021). Capture of Mouse and Human Stem Cells with Features of Formative Pluripotency. Cell Stem Cell 28, 453-471 e458.
Kurek, D., Neagu, A., Tastemel, M., Tuysuz, N., Lehmann, J., van de Werken, H. J. G., Philipsen, S., van der Linden, R., Maas, A., van, I. W. F. J., et al. (2015). Endogenous WNT signals mediate BMP-induced and spontaneous differentiation of epiblast stem cells and human embryonic stem cells. Stem Cell Reports 4, 114-1661 128.
Li, Z., Duan, X., An, X., Feng, T., Li, P., Li, L., Liu, J., Wu, P., Pan, D., Du, X., et al. (2018). Efficient RNA-guided base editing for disease modeling in pigs. Cell Discov. 4, 64.
Lindsly, S., Chen, C., Liu, S., Ronquist, S., Dilworth, S., Perlman, M., and Rajapakse, I. (2021). 4DNvestigator: Time Series Hi-C and RNA-seq Data Analysis Toolbox. bioRxiv, 2020.2001.2008.898387.
Lyu, X., Rowley, M. J., and Corces, V. G. (2018). Architectural Proteins and Pluripotency Factors Cooperate to Orchestrate the Transcriptional Response of hESCs to Temperature Stress. Mol. Cell 71, 940-955 e947.
Martin, G. R. (1981). Isolation of a pluripotent cell line from early mouse embryos cultured in medium conditioned by teratocarcinoma stem cells. Proc. Natl. Acad. Sci. U.S.A 78, 7634-7638.
Mclaughlin, K., Flyamer, I. M., Thomson, J. P., Mjoseng, H. K., Shukla, R., Williamson, I., Grimes, G. R., Illingworth, R. S., Adams, I. R., Pennings, S., et al. (2019). DNA Methylation Directs Polycomb-Dependent 3D Genome Re-organization in Naive Pluripotency. Cell Rep. 29, 1974-1985 e1976.
Nir, G., Farabella, I., Perez Estrada, C., Ebeling, C. G., Beliveau, B. J., Sasaki, H. M., Lee, S. D., Nguyen, S. C., McCole, R. B., Chattoraj, S., et al. (2018). Walking along chromosomes with super-resolution imaging, contact maps, and integrative modeling. PLOS Genet. 14, e1007872. Notarianni, E., Laurie, S., Moor, R. M., and Evans, M. J. (1990). Maintenance and differentiation in culture of pluripotential embryonic cell lines from pig blastocysts. J. Reprod. Fertil. Suppl. 41, 51-56.
Park, J. K., Kim, H. S., Uh, K. J., Choi, K. H., Kim, H. M., Lee, T., Yang, B. C., Kim, H. J., Ka, H. H., Kim, H., et al. (2013). Primed pluripotent cell lines derived from various embryonic origins and somatic cells in pig. PLOS One 8, e52481.
Shukla, R., Mjoseng, H. K., Thomson, J. P., Kling, S., Sproul, D., Dunican, D. S., Ramsahoye, B., Wongtawan, T., Treindl, F., Templin, M. F., et al. (2020). Activation of transcription factor circuitry in 2i-induced ground state pluripotency is independent of repressive global epigenetic landscapes. Nucleic Acids Res. 48, 7748-7766.
Tan, L., Xing, D., Chang, C. H., Li, H., and Xie, X. S. (2018). Three-dimensional genome structures of single diploid human cells. Science 361, 924-928.
Tesar, P. J., Chenoweth, J. G., Brook, F. A., Davies, T. J., Evans, E. P., Mack, D. L., Gardner, R. L., and Mckay, R. D. (2007). New cell lines from mouse epiblast share defining features with human embryonic stem cells. Nature 448, 196-199.
Theunissen, T. W., Powell, B. E., Wang, H., Mitalipova, M., Faddah, D. A., Reddy, J., Fan, Z. P., Maetzel, D., Ganz, K., Shi, L., et al. (2014). Systematic identification of culture conditions for induction and maintenance of naive human pluripotency. Cell Stem Cell 15, 471-487.
Thomson, J. A., Itskovitz-Eldor, J., Shapiro, S. S., Waknitz, M. A., Swiergiel, J. J., Marshall, V. S., and Jones, J. M. (1998). Embryonic stem cell lines derived from human blastocysts. Science 282, 1145-1147.
Valdez Magana, G., Rodriguez, A., Zhang, H., Webb, R., and Alberio, R. (2014). Paracrine effects of embryo derived FGF4 and BMP4 during pig trophoblast elongation. Dev. Biol. 387, 15-27.
Vassiliev, I., Vassilieva, S., Beebe, L. F., Harrison, S. J., McIlfatrick, S. M., and Nottle, M. B. (2010). In vitro and in vivo characterization of putative porcine embryonic stem cells. Cell. Reprogram. 12. 223-230.
Wang, X., Xiang, Y., Yu, Y., Wang, R., Zhang, Y., Xu, Q., Sun, H., Zhao, Z. A., Jiang, X., Wang, X., et al. (2021). Formative pluripotent stem cells show features of epiblast cells poised for gastrulation. Cell Res. 10.1038/s41422-021-00477-x.
Wang, M., Liu, X., Chang, G., Chen, Y., An, G., Yan, L., Gao, S., Xu, Y., Cui, Y., Dong, J., et al. (2018). Single-Cell RNA Sequencing Analysis Reveals Sequential Cell Fate Transition during Human Spermatogenesis. Cell Stem Cell 23, 599-614 e594.
Whalen, S., Truty, R. M., and Pollard, K. S. (2016). Enhancer-promoter interactions are encoded by complex genomic signatures on looping chromatin. Nat. Genet. 48, 488-496.
Xie, J., Ge, W., Li, N., Liu, Q., Chen, F., Yang, X., Huang, X., Ouyang, Z., Zhang, Q., Zhao, Y., et al. (2019). Efficient base editing for multiple genes and loci in pigs using base editors. Nat. Commun. 10, 2852.
Yan, S., Tu, Z., Liu, Z., Fan, N., Yang, H., Yang, S., Yang, W., Zhao, Y., Ouyang, Z., Lai, C., et al. (2018). A Huntingtin Knockin Pig Model Recapitulates Features of Selective Neurodegeneration in Huntington's Disease. Cell 173, 989-1002 e1013.
Ying, Q. L., Wray, J., Nichols, J., Batlle-Morera, L., Doble, B., Woodgett, J., Cohen, P., and Smith, A. (2008). The ground state of embryonic stem cell self-renewal. Nature 453, 519-523.
Yu, L., Wei, Y., Sun, H. X., Mahdi, A. K., Pinzon Arteaga, C. A., Sakurai, M., Schmitz, D. A., Zheng, C., Ballard, E. D., Li, J., et al. (2021). Derivation of Intermediate Pluripotent Stem Cells Amenable to Primordial Germ Cell Specification. Cell Stem Cell 28, 550-567 e512.
Yuan, Y., Park, J., Tian, Y., Choi, J., Pasquariello, R., Alexenko, A. P., Dai, A., Behura, S. K., Roberts, R. M., and Ezashi, T. (2019). A six-inhibitor culture medium for improving naive-type pluripotency of porcine pluripotent stem cells. Cell Death Discov. 5. 10⁴.
Zhang, X., Xue, B., Li, Y., Wei, R., Yu, Z., Jin, J., Zhang, Y., and Liu, Z. (2019). A novel chemically defined scrum- and feeder-free medium for undifferentiated growth of porcine pluripotent stem cells. J. Cell. Physiol. 10.1002/jcp.28185.

1.-35. (canceled)

36. A pluripotent stem cell, having a pluripotency of pig pre-gastrulation Epiblast cells, expressing one or more pluripotency markers and one or more Epiblast markers, and is capable of stable passage;
1 preferably, the pluripotent stem cell is derived from pig embryo.

37. The pluripotent stem cell according to claim 36, wherein the one or more pluripotency markers are selected from POU5F1, NANOG, SOX2, SSEA1, SSEA4, TRA-1-81, TRA-1-60 and any combination thereof;
preferably, the pluripotent stem cell expresses one or more of POU5F1, NANOG and SOX2;
preferably, the pluripotent stem cell expresses one or more of SSEA1, SSEA4, TRA-1-81 and TRA-1-60;
preferably, the pluripotent stem cell expresses one or more of POU5F1, NANOG, SOX2, SSEA1, SSEA4, TRA-1-81 and TRA-1-60.

38. The pluripotent stem cell according to claim 36, wherein the one or more Epiblast markers are selected from NANOG, TDGF1, ETV4, GDF3, NODAL, PRDM14, ETV5, CACHD1 and any combination thereof;
preferably, the pluripotent stem cell expresses one or more of NANOG, TDGF1, ETV4, GDF3 and NODAL;
preferably, the pluripotent stem cell expresses one or more of NANOG, TDGF1, ETV4, GDF3, NODAL, PRDM14, ETV5 and CACHD1.

39. The pluripotent stem cell according to claim 36, which does not express or expresses at least one Hypoblast marker at a low level; or, the pluripotent stem cell expresses at least one Hypoblast marker at a level that is lower than that in a pig embryonic Hypoblast cell of E8 to E10 (e.g., E8, E9, or E10);
preferably, the Hypoblast marker is selected from IGF1, SRC, HNF4A, BMP2, SOX17, PDGFRA, NID2, RSPO3, GATA4, LAMA1 or any combination thereof;
preferably, the pluripotent stem cell does not express or express one or more of HNF4A, SOX17 and GATA4 at a low level;
preferably, the pluripotent stem cell expresses at least one (e.g., at least two or all) selected from the following genes at a level that is lower than that in a pig embryonic Hypoblast cell of E8 to E10 (e.g., E8, E9, or E10): HNF4A, SOX17 and GATA4;
preferably, the pluripotent stem cell does not express or expresses at least one gastrulation marker at a low level; or, the pluripotent stem cell expresses at least one gastrulation marker at a level that is lower than that in a pig embryonic Ectoderm cell of E11 to E14 (for example, E11, E12, E13 or E14);
preferably, the gastrulation marker is selected from EOMES, WNT5A, BMP4, LEF1, HAND1 and any combination thereof;
preferably, the pluripotent stem cell does not express or expresses one or more of EOMES, WNT5A, BMP4, LEF1 and HAND1 at a low level;
preferably, the pluripotent stem cell expresses at least one (for example, at least 2, at least 3, at least 4 or all) selected from the following genes at a level that is lower than that in a pig embryonic Ectoderm cell of E11 to E14 (for example, E11, E12, E13 or E14): EOMES, WNT5A, BMP4, LEF1 and HAND1.

40. The pluripotent stem cell according to claim 36, wherein the pluripotent stem cell shows at least about a 2-fold increase in the expression level of at least one (for example, at least 2, at least 5, at least 10, at least 15, at least 20 or all) gene selected from the following in relative to that in a human embryonic stem cell: ADPRM, FRG1, GAS2, HK3, NCAN, POU5F1B, ZFP2, CLDND2, CRK, DMP1, GATD3B, H3F3A, IRF8, ITGA4, KRT14, MPC1, MSH4, NDE1, PBX2, PRKY, RGL2, SOX10 and VHLL;
preferably, the pluripotent stem cell shows at least about a 2-fold decrease in the expression level of at least one (for example, at least 2, at least 5, at least 10, at least 15 or all) gene selected from the following in relative to that in a human embryonic stem cell: ABCC4, ADCY2, AK2, AKT1, BMP2, CD46, CDH3, DNM1, DPPA4, ETS1, GAB2, ID2, KDR, MMP24, TGFB1, VGLL3, ZNF195 and ZNF519;
preferably, the expression level of at least one (for example, at least 2, at least 5, at least 10, at least 15, at least 20, at least 25, at least 30, at least 35, at least 40, at least 45, at least 50, at least 55, at least 60, at least 65, at least 70 or all) gene selected from Table 1 of is increased in relative to that in a porcine embryonic fibroblast;
preferably, the expression level of at least one (for example, at least 2, at least 5, at least 10, at least 15, at least 20 or all) gene selected from the following is increased in relative to that in a porcine embryonic fibroblast: ZSCAN21, LIN28B, MYCN, SALL4, CDH1, DNMT3B, ZFP42, SOX2, UTF1, PRDM14, LEFTY2, OTX2, LIN28A, ACVR2B, HESX1, FZD5, PPP1R1A, VMO1, NANOG, KRT8, KRT18 and EPCAM;
preferably, the human embryonic stem cell is a conventional human embryonic stem cell (conventional hESC) or a human embryonic stem cell in a primed state.

41. The pluripotent stem cell according to claim 36, wherein the pluripotent stem cell comprises genes with co-variation between regulatory potential score (RPS) and Expression in relative to a porcine embryonic fibroblast (pEF), wherein the gene is selected from at least one (for example, at least 2, at least 5, at least 10, at least 15, at least 20, at least 25, at least 30, at least 35, at least 40, at least 45, at least 50, at least 55, at least 60, at least 65, at least 70 or all) gene in Table 1;
preferably, at least one (for example, at least 2, at least 3, at least 4, at least 5 or all) transcription factor specifically interacts with an enhancer selected from the following in the genome of the pluripotent stem cell in relative to porcine embryonic fibroblast: OTX2, LIN28A, NANOG, PRDM14, SALL4, UTF1, ZFP42, CDH1, DNMT3B and LEFTY2;
preferably, the specific interaction with the enhancer refers to that the transcription factor interacts with the enhancer as determined by the high-deep in situ high-throughput chromatin conformation capture (Hi-C) sequencing, and that the above interaction is absent or relatively rare in the porcine embryonic fibroblasts.

42. The pluripotent stem cell according to claim 36, wherein the pluripotent stem cell has the capacity to differentiate into a cell of any one of endoderm, ectoderm and mesoblast;
preferably, the pluripotent stem cell is capable of forming a dome-shaped clonal morphology;
preferably, the pluripotent stem cell is capable of stable passage for at least 10 times, at least 20 times, at least 30 times, at least 40 times, at least 50 times, at least 100 times, at least 150 times, at least 200 times or more.

43. The pluripotent stem cell according to claim 36, wherein the pluripotent stem cell is derived from pre-gastrulation Epiblast of a pig embryo;
preferably, the pluripotent stem cell is derived from E8-E10 (for example, E8, E9 or E10) pre-gastrulation Epiblast of a pig embryo;
preferably, the pluripotent stem cell is derived from E10 Epiblast of a pig embryo;
preferably, the pluripotent stem cell is a cell line;
preferably, the pluripotent stem cell is an Epiblast stem cell.

44. An isolated cell population comprising the pluripotent stem cell according to claim 36.

45. The cell population according to claim 44, wherein at least 50% (for example, at least 60%, at least 70%, at least 75%, at least 80%, at least 85%, at least 90%, at least 95%, at least 98%, at least 99% or about 100%) cells in the cell population are the pluripotent stem cells;
preferably, at least 50% (e.g., at least 60%, at least 70%, at least 75%, at least 80%, at least 85%, at least 90%, at least 95%, at least 98%, at least 99% or about 100%) cells in the cell population express one or more markers selected from ETV5, NANOG, ETV4, NODAL and GDF3;
preferably, about 100% cells in the cell population express ETV5, NANOG, ETV4, NODAL and GDF3.

46. A genetically modified pluripotent stem cell obtained by genetically modifying the pluripotent stem cell according to claim 36.

47. The genetically modified pluripotent stem cell of claim 46, wherein the genetic modification comprises genome editing, comprising for example, nucleic acid fragment deletion, gene modification, gene knockout, gene product expression alteration, repair of mutations, polynucleotide insertion, single base mutation or any combination thereof;
preferably, the genome editing comprises gene insertion, gene knock-in, gene knockout, gene mutation (such as single base mutation) or any combination thereof;
preferably, the genetically modified pluripotent stem cell comprises at least two (for example, at least three) genetic modifications or has undergone at least twice (for example, at least three times) genetic modification.

48. A method for producing a genetically modified pluripotent stem cell, which comprises genetically modifying the pluripotent stem cell according to claim 36;
preferably, the genetic modification comprises genome editing, comprising for example, nucleic acid fragment deletion, gene modification, gene knockout, gene product expression alteration, repair of mutations, polynucleotide insertion, single base mutation or any combination thereof;
preferably, the genome editing comprises gene insertion, gene knock-in, gene knockout, gene mutation (such as single base mutation) or any combination thereof.

49. Any of the following methods:
(1) a method for producing a pig embryo;
(2) a method for producing a cloned pig;
(3) a method for producing a cell, a tissue or an organ (for example, an organoid) in vitro;
(4) a method for preparing a disease model and/or a drug screening model.

50. The method according to claim 49, wherein the method for producing a pig embryo comprises establishing an embryo by a nuclear transfer process, wherein a nucleus of a pluripotent stem cell, having a pluripotency of pig pre-gastrulation Epiblast cells, expressing one or more pluripotency markers and one or more Epiblast markers, and is capable of stable passage;
preferably, the pluripotent stem cell is derived from pig embryo, is transferred into an enucleated porcine oocyte or egg cell.

51. The method according to claim 49, wherein the method for producing a cloned pig uses a pluripotent stem cell, having a pluripotency of pig pre-gastrulation Epiblast cells, expressing one or more pluripotency markers and one or more Epiblast markers, and is capable of stable passage;
preferably, the pluripotent stem cell is derived from pig embryo;
preferably, the method comprises:
establishing an embryo by a nuclear transfer process, wherein a nucleus of the pluripotent stem cell is transferred into an enucleated porcine oocyte or an egg cell; and
transferring the embryo into a recipient host for gestation;
preferably, the method further comprises culturing the nuclear donor cell under the condition of inducing differentiation prior to nuclear transfer.

52. The method according to claim 49, wherein the method for producing a cell, a tissue or an organ (for example, an organoid) in vitro uses a pluripotent stem cell, having a pluripotency of pig pre-gastrulation Epiblast cells, expressing one or more pluripotency markers and one or more Epiblast markers, and is capable of stable passage;
preferably, the pluripotent stem cell is derived from pig embryo;
preferably, the method comprises culturing the pluripotent stem cell under conditions that allow pluripotent stem cells to differentiate;
preferably, the cell comprises endodermal, ectodermal or mesodermal cells.

53. The method according to claim 49, wherein the method for preparing a disease model and/or a drug screening model uses a pluripotent stem cell, having a pluripotency of pig pre-gastrulation Epiblast cells, expressing one or more pluripotency markers and one or more Epiblast markers, and is capable of stable passage;
preferably, the pluripotent stem cell is derived from pig embryo, or a cell, a tissue or an organ (for example, an organoid) produced by the pluripotent stem cell, the cell population or the genetically modified pluripotent stem cell in vitro.

54. A pig embryo produced by the method of claim 50.

55. A disease model and/or a drug screening model, comprising the pluripotent stem cell according to claim 36, or a cell, a tissue or an organ (for example, an organoid) produced by the pluripotent stem cell, the cell population or the genetically modified pluripotent stem cell in vitro.
